(19) United States
(12) Patent Application Publication
(10) Pub. No.: US 2024/0350563 A1
(43) Pub. Date: Oct. 24, 2024

(54) BIOTHERAPEUTIC ENTEROCOCCUS ISOLATES

(71) Applicants: Hudson Institute of Medical Research, Clayton (AU); Monash University, Clayton (AU); Monash Health, Clayton (AU)

(72) Inventors: Samuel Charles Forster, Bittern (AU); Edward Mark Giles, Alphington (AU); Gemma Louise D'adamo, Bellarine (AU)

(21) Appl. No.: 18/683,452

(22) PCT Filed: Sep. 20, 2022
(86) PCT No.: PCT/AU2022/051133

§ 371 Date: Feb. 13, 2024

(30) Foreign Application Priority Data
Sep. 20, 2021 (AU) 2021903022

(51) Int. Cl. A61K 35/744; C12N 1/20; C12R 1/46

(52) U.S. Cl. CPC A61K 35/744; C12N 1/205; C12R 2001/46

(57) ABSTRACT

The present invention relates to new strains of Enterococcus sp., in particular, inflammatory and non-inflammatory strains of Enterococcus sp., useful for bacterio therapy.

### FIELD OF THE INVENTION

The present invention relates to new strains of Enterococcus sp. useful for bacteriotherapy.

### BACKGROUND OF THE INVENTION

The human intestinal microbiota consists of trillions of microorganisms including at least 100 prevalent and at least 1000 less common bacterial species, harbouring over 100-fold more genes than those present in the human genome. The intestinal microbiota is composed predominantly of bacteria, yet also contains archaea, protozoa, and viruses. The microbiota performs vital functions essential to health maintenance, including food processing, digestion of complex indigestible polysaccharides and synthesis of vitamins, and it secretes bioactive metabolites with diverse functions, ranging from inhibition of pathogens, metabolism of toxic compounds to modulation of host metabolism.

Inflammatory bowel disease (IBD) is an increasingly prevalent, currently incurable condition believed to be caused by an abnormal immune response to the resident gut microbiome in genetically susceptible patients (Graham and Xavier, 2020). Affecting both adults and children, adult cohorts are frequently confounded by co-morbidities, disease course, existing medications and lifestyle factors typically absent in newly diagnosed paediatric populations. High-throughput sequencing of both adult and paediatric patient cohorts has now provided detailed taxonomic understanding of microbiome composition in IBD (Schirmer et al., 2019). However, existing studies are dominated by faecal sampling from adult cohorts, with phenotypic investigation of human bacteria largely limited to genomic predictions and correlative associations (reviewed by Ni et al., 2017). The variation in disease state across the gastrointestinal tract, coupled with confounding factors in adult patient cohorts, necessitates detailed host and microbiome investigation of mucosal samples in paediatric patient cohorts.

Studies have shown that bacteriotherapy using beneficial bacterial isolates can be used to treat and/or prevent diseases/disorders such as irritable bowel syndrome, inflammatory bowel diseases, ulcers, or stomach cancer. Thus, there is the need for the identification of further bacteria for use in bacteriotherapy.

### SUMMARY OF THE INVENTION

The present inventors have isolated a new Enterococcus sp. useful for bacteriotherapy.

The present inventors have identified non-inflammatory strains of Enterococcussp. Thus, in an aspect the present invention provides a method of reducing or preventing gastrointestinal tract mucosal inflammation in a subject, the method comprising administering to the subject a therapeutically effective amount of a biotherapeutic composition comprising a non-inflammatory strain of Enterococcus sp.

In an embodiment, the non-inflammatory strain comprises a 16s ribosomal RNA (rRNA) gene having a nucleotide sequence as shown in any one of SEQ ID NO's 1 to 44, or a nucleotide sequence at least 90% identical to one or more of SEQ ID NO's 1 to 44. Examples of such strains include, but are not limited to;
i) Enterococcus sp. CC00149 deposited under V19/018754 on 9 Sep. 2019 at the National Measurement Institute, Australia,
ii) Enterococcus sp. CC00259 deposited under V19/018755 on 9 Sep. 2019 at the National Measurement Institute, Australia, and
iii) Enterococcus sp. CC00620 deposited under V21/013048 on 29 Jun. 2021 at the National Measurement Institute, Australia.

In a further aspect, the present invention provides a method of treating or preventing a dysbiosis of the gastrointestinal tract in a subject, the method comprising administering to the subject a therapeutically effective amount of a biotherapeutic composition comprising a strain of Enterococcus sp. which comprises a 16s ribosomal RNA (rRNA) gene having a nucleotide sequence as shown in any one of SEQ ID NO's 1 to 74, or a nucleotide sequence at least 90% identical to one or more of SEQ ID NO's 1 to 74.

In an embodiment, the Enterococcus sp. is a non-inflammatory strain and comprises a 16s ribosomal RNA (rRNA) gene having a nucleotide sequence as shown in any one of SEQ ID NO's 1 to 44, or a nucleotide sequence at least 90% identical to one or more of SEQ ID NO's 1 to 44.

In an alternate embodiment, the Enterococcus sp, is an inflammatory strain and comprises a 16s ribosomal RNA (rRNA) gene having a nucleotide sequence as shown in any one of SEQ ID NO's 45 to 74, or a nucleotide sequence at least 90% identical to one or more of SEQ ID NO's 45 to 74. Examples of such strains include, but are not limited to;
i) Enterococcus sp. CC00064 deposited under V21/013046 on 29 Jun. 2021 at the National Measurement Institute, Australia,
ii) Enterococcus sp. CC00619 deposited under V21/013047 on 29 Jun. 2021 at the National Measurement Institute, Australia,
iii) Enterococcus sp. CC00262 deposited under V20/006238 on 18 Mar. 2020 at the National Measurement Institute, Australia, and
iv) Enterococcus sp. CC0002 deposited under V21/014119 on 20 Jul. 2021 at the National Measurement Institute, Australia.

In an embodiment, the dysbiosis and/or inflammation is associated with one or more of inflammatory bowel disease (IBD), pouchitis, irritable bowel syndrome (IBS), an enteric bacterial infection, a metabolic disease, a neuropsychiatric disorder, an autoimmune disease, an allergic disorder, hepatic encephalopathy, or a cancer. In an embodiment, the IBD is ulcerative colitis (UC) or Crohn's disease.

In an embodiment, the composition further comprises a prebiotic.

In an embodiment, the composition further comprises a carrier.

In an embodiment, the composition further comprises insoluble fiber, a buffer, an osmotic agent, an antifoaming agent, and/or a preservative.

In an embodiment, wherein the composition comprises a chemostat medium.

In an embodiment, the composition is a saline composition.

In an embodiment, the composition is administered orally or rectally.

In an embodiment, the composition further comprises a stabiliser and/or a cryoprotectant.

In an embodiment, the composition is freeze dried.

In an embodiment, the composition is the form of a capsule, a tablet, or an enema. In an embodiment, the capsule or tablet is enteric-coated, pH dependant, slow-release, and/or gastro-resistant.

In an embodiment, the strain is present in the composition at about 10³ to about 10¹³, or about 10⁴ to about 10¹², or about 10⁵ to about 10¹¹, or about 10⁶ to about 10¹⁰, or about 10⁷ to about 10⁹, cfu per gram.

In an embodiment, the subject is a human.

In a further aspect, the present invention provides an isolated non-inflammatory strain of Enterococcus sp. In an embodiment, the non-inflammatory strain comprises a 16s ribosomal RNA (rRNA) gene having a nucleotide sequence as shown in any one of SEQ ID NO's 1 to 44, or a nucleotide sequence at least 90% identical to one or more of SEQ ID NO's 1 to 44.

In another aspect, the present invention provides an isolated strain of Enterococcus sp. which comprises a 16s ribosomal RNA (rRNA) gene having a nucleotide sequence as shown in any one of SEQ ID NO's 1 to 74, or a nucleotide sequence at least 90% identical to one or more of SEQ ID NO's 1 to 74. In an embodiment, the Enterococcus sp, strain is an inflammatory strain and comprises a 16s ribosomal RNA (rRNA) gene having a nucleotide sequence as shown in any one of SEQ ID NO's 45 to 74, or a nucleotide sequence at least 90% identical to one or more of SEQ ID NO's 45 to 74.

In a further aspect, the present invention provides a composition comprising at least one strain of the invention. In an embodiment, the composition is a biotherapeutic composition.

In an aspect, the present invention provides a method of preparing a biotherapeutic composition, the method comprising:
i) culturing at least one strain of Enterococcus sp. of the invention, and
ii) mixing the Enterococcus sp. obtained in i) with a pharmaceutically acceptable carrier.

In a further aspect, the present invention provides a method of analysing the microbiome of the gastrointestinal tract of a subject, the method comprising;
i) obtaining a sample comprising bacteria from the gastrointestinal tract of the subject,
ii) analysing the sample for the presence of a strain of Enterococcus sp. of the invention.

In an embodiment, DNA of, or extracted from, the sample is analysed.

In an embodiment, the sample is analysed by DNA amplification and/or DNA hybridization.

In an embodiment, the presence of a strain of Enterococcus sp. which comprises a 16s ribosomal RNA (rRNA) gene having a nucleotide sequence as shown in any one of SEQ ID NO's 45 to 74, or a nucleotide sequence at least 90% identical to one or more of SEQ ID NO's 45 to 74, suggests that the gastrointestinal tract is inflamed, or that a previously detected dysbiosis and/or gastrointestinal tract inflammation is at least partly due to the strain.

In an embodiment, the presence of a strain of Enterococcus sp. which comprises a 16s ribosomal RNA (rRNA) gene having a nucleotide sequence as shown in any one of SEQ ID NO's 1 to 44, or a nucleotide sequence at least 90% identical to one or more of SEQ ID NO's 1 to 44, suggests that the gastrointestinal tract is not inflamed, and/or that the gastrointestinal tract comprises non-inflammatory strains of Enterococcus sp.

In an embodiment, a non-inflammatory strain of the invention is, or can be, detected using the primer pairs;

i) CGAGGTACACCGTAAGTTGTTGCT
(SEQ ID NO: 75)
and
(SEQ ID NO: 76)
ACCAGGACACGTAGACTTCACAGT,
or
ii)
(SEQ ID NO: 77)
TGGCCCAAAGATACGTGTTCCAC
and
(SEQ ID NO: 78)
CGAACAAAATCAGGAGCTCGTCGT.

In an embodiment, an inflammatory strain of the invention is, or can be, detected using the primer pairs;

i)
(SEQ ID NO: 79)
ACTGCTGAGTGCCCACGCTT
and
(SEQ ID NO: 80)
GAGGCACTTGCTAGCGTCACAC,
ii)
(SEQ ID NO: 81)
TGCGTGTAGGCATCATCCGTGA
and
(SEQ ID NO: 82)
GACACAGCGGCATCAGCAAGT,
iii)
(SEQ ID NO: 83)
CCTTTACCATGGCCGTGTAGC
and
(SEQ ID NO: 84)
CCATGAATGGTGTTCCTCCTTCTC,
or
iv)
(SEQ ID NO: 85)
AAACTTGCTGATGCCGCTGT
and
(SEQ ID NO: 86)
CCGCCTTTACCGATAACCATACC.

Also provided is the use of at least one strain of Enterococcus sp. of the invention for manufacture of a medicament for treating or preventing a dysbiosis of the gastrointestinal tract.

Also provided is the use of at least one non-inflammatory strain of Enterococcus sp for manufacture of a medicament for reducing or preventing gastrointestinal tract mucosal inflammation in a subject.

Also provided is at least one strain of Enterococcus sp. of the invention for use in treating or preventing a dysbiosis of the gastrointestinal tract.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Workflow for direct microbial culturing and sequencing from patient derived intestinal biopsy samples. Workflow for the culturing of bacterial communities directly from mucosal samples, to minimise the amount of eukaryotic DNA present.

FIG. 2. A subset of IBD samples demonstrate clade specific bacterial enrichment. Differences in relative proportion of key Enterococcus clades in the control and IBD patients (P<0.05; KS) highlights the existence of a distinct subset of IBD patients.

FIG. 3. Fewer virulence genes are associated with the isolates achieving high cell cytotoxicity in stimulated Caco2 cells. The number of virulence genes in the isolates of interest from each clade was assessed, using a percent identity cut-off of >70% (P<0.01; Mann-Whitney U test).

FIG. 4. In vitro phenotypic validation of candidate bacteria demonstrates a clade specific difference in cell cytotoxicity. Cell cytotoxicity at 24 hours by LDH assay in Caco2 intestinal epithelial cells stimulated with one of twelve isolates representing the IBD clade (six isolates on the right) and control clade at a multiplicity of infection (MOI) of 10:1 (n=3 biological replicates, mean±SEM). Bacterial clades are represented by the phylogenetic tree below the graph, depicting the relationship between isolates. Statistics represent a comparison between the clade associated with IBD vs control clade (P=0.0006; Ratio paired t-test). SEM images of each isolate also allow for morphological visualisation of each candidate bacteria.

FIG. 5. Scanning electron microscopy (SEM) of the 12 isolates used to stimulate Caco2 cells. a. The six isolates in the control-associated clade, that cause greater cell cytotoxicity when stimulating Caco2 cells. b, The six isolates in the IBD-enriched clade that cause less cell cytotoxic activity when stimulating Caco2 cells.

FIG. 6. Viability of bacterial isolates is essential to achieve the cytotoxic phenotype. A representative isolate from each of the clades under investigation was selected (CC00064 and CC00149) and used to stimulate Caco2 cells, following heat inactivation of the isolates for 60 minutes at 99°C. Concentrations of both 1 mg/ml and 1 μg/ml of heat killed bacteria were used and cell cytotoxicity was assessed at 0, 4, 6, 8 and 24 hours post-stimulation. Ratio paired t-test was used, no significant differences noted. n=3 biological replicates.

FIG. 7. Significantly differentially expressed genes identified following stimulation of Caco2 epithelial cells with CC00064 and CC00149. a, 4 hours post stimulation. b, 8 hours post stimulation. c, 24 hours post stimulation.

FIG. 8. In vitro clade specific gene signatures present in inflamed patient derived biopsies. a, Genes for which there was significant differential expression in the in vitro Caco2 experiment between isolates CC00149 and CC00064 at 8 hours were compared to the differential expression results between the inflamed and non-inflamed mucosal samples from each intestinal region. Vertical lines represent genes in the set, which showed upregulation in strain CC00149, compared to CC00064 (weighted by log2 fold change). Genes are positioned according to the moderated t-statistic in the clinical group of interest, with highly upregulated genes positioned on the far right, while downregulated genes appear on the far left. Overall gene set enrichment is represented by the enrichment line shown above. b, Heat map showing all genes that were significantly differentially expressed between CC00149 and CC00064 at 8 hours post stimulation, that were also significantly changed in the same direction, between the molecularly inflamed compared to non-inflamed samples, within any of the three clinical sample sites. The top 6 genes were all reduced, and the rest increased with the exception of LMO4 and EPHA2 in the terminal ileum. Significance (P<0.05) is represented by the asterix (*) in the box. Values displayed are log2 fold changes, truncated to ±3.

FIG. 9. Cycle threshold (CT) values of quantitative real time PCR for the cytotoxic specific primers (A) and non-cytotoxic specific primers (B) demonstrating clade specificity in both primer sets.

### KEY TO THE SEQUENCE LISTING

SEQ ID NO:1-16s rRNA nucleotide sequence of Enterococcus sp. isolate CC00149
SEQ ID NO:2-16s rRNA nucleotide sequence of Enterococcus sp. isolate CC00259
SEQ ID NO:3-16s rRNA nucleotide sequence of Enterococcus sp. isolate CC00261
SEQ ID NO:4-16s rRNA nucleotide sequence of Enterococcus sp. isolate CC00260
SEQ ID NO:5-16s rRNA nucleotide sequence of Enterococcus sp. isolate CC00620
SEQ ID NO:6-16s rRNA nucleotide sequence of Enterococcus sp. isolate CC00152
SEQ ID NO:7-16s rRNA nucleotide sequence of Enterococcus sp. isolate mc056tix_3d_mc056_57_d3
SEQ ID NO:8-16s rRNA nucleotide sequence of Enterococcus sp. isolate mc022mx_e1
SEQ ID NO:9-16s rRNA nucleotide sequence of Enterococcus sp. isolate mc056tix_3h_mc056_57_h3
SEQ ID NO:10-16s rRNA nucleotide sequence of Enterococcus sp. isolate mc056tix_3f_mc056_57_f3
SEQ ID NO:11-16s rRNA nucleotide sequence of Enterococcus sp. isolate mc036cna_f03
SEQ ID NO:12-16s rRNA nucleotide sequence of Enterococcus sp. isolate mc056tix_4a_mc056_57_a4
SEQ ID NO:13-16s rRNA nucleotide sequence of Enterococcus sp. isolate mc056tix_3e_mc056_57_e3
SEQ ID NO:14-16s rRNA nucleotide sequence of Enterococcus sp. isolate mc056cix_8d_mc056_57_d8
SEQ ID NO:15-16s rRNA nucleotide sequence of Enterococcus sp. isolate mc056cix_8e_mc056_57_e8
SEQ ID NO:16-16s rRNA nucleotide sequence of Enterococcus sp. isolate mc056tix_3a_mc056_57_a
SEQ ID NO:17-16s rRNA nucleotide sequence of Enterococcus sp. isolate mc056tix_1c_mc056_57_c1
SEQ ID NO:18-16s rRNA nucleotide sequence of Enterococcus sp. isolate mc056tix_3b_mc056_57_b3
SEQ ID NO:19-16s rRNA nucleotide sequence of Enterococcus sp. isolate mc036rna2_h03
SEQ ID NO:20-16s rRNA nucleotide sequence of Enterococcus sp. isolate mc056cix_5f_mc056_57_f5
SEQ ID NO:21-16s rRNA nucleotide sequence of Enterococcus sp. isolate mc056cix_6d_mc056_57_d6
SEQ ID NO:22-16s rRNA nucleotide sequence of Enterococcus sp. isolate mc056tix_3g_mc056_57_g3
SEQ ID NO:23-16s rRNA nucleotide sequence of Enterococcus sp. isolate mc056cix_8c_mc056_57_c8
SEQ ID NO:24-16s rRNA nucleotide sequence of Enterococcus sp. isolate mc056cix_7h_mc056_57_h7
SEQ ID NO:25-16s rRNA nucleotide sequence of Enterococcus sp. isolate mc056tix_2b_mc056_57_b2
SEQ ID NO:26-16s rRNA nucleotide sequence of Enterococcus sp. isolate mc017tn_x_b6
SEQ ID NO:27-16s rRNA nucleotide sequence of Enterococcus sp. isolate mc017cn_x_d3
SEQ ID NO:28-16s rRNA nucleotide sequence of Enterococcus sp. isolate mc017cn_x_d2
SEQ ID NO:29-16s rRNA nucleotide sequence of Enterococcus sp. isolate mc026cna2_e06
SEQ ID NO:30-16s rRNA nucleotide sequence of Enterococcus sp. isolate mc026rna2_g06
SEQ ID NO:31-16s rRNA nucleotide sequence of Enterococcus sp. isolate mc059rn_fx_g10
SEQ ID NO:32-16s rRNA nucleotide sequence of Enterococcus sp. isolate mc017rn_x_f4
SEQ ID NO:33-16s rRNA nucleotide sequence of Enterococcus sp. isolate mc017rn_12f_a
SEQ ID NO:34-16s rRNA nucleotide sequence of Enterococcus sp. isolate mc017tn_12a_ma
SEQ ID NO:35-16s rRNA nucleotide sequence of Enterococcus sp. isolate mc017tn_1a_ma
SEQ ID NO:36-16s rRNA nucleotide sequence of Enterococcus sp. isolate mc017rn_12e_ma
SEQ ID NO:37-16s rRNA nucleotide sequence of Enterococcus sp. isolate mc017cn_12c_ma
SEQ ID NO:38-16s rRNA nucleotide sequence of Enterococcus sp. isolate mc026rnm2_a08
SEQ ID NO:39-16s rRNA nucleotide sequence of Enterococcus sp. isolate mc017tn_x_a1
SEQ ID NO:40-16s rRNA nucleotide sequence of Enterococcus sp. isolate mc127rifx b11
SEQ ID NO:41-16s rRNA nucleotide sequence of Enterococcus sp. isolate mc026tnm2_c07
SEQ ID NO:42-16s rRNA nucleotide sequence of Enterococcus sp. isolate mc097cnfx_h6
SEQ ID NO:43-16s rRNA nucleotide sequence of Enterococcus sp. isolate mc127tnfx_c2
SEQ ID NO:44-16s rRNA nucleotide sequence of Enterococcus sp. isolate mc127rifx_c10
SEQ ID NO:45-16s rRNA nucleotide sequence of Enterococcus sp. isolate CC00064
SEQ ID NO:46-16s rRNA nucleotide sequence of Enterococcus sp. isolate CC00262
SEQ ID NO:47-16s rRNA nucleotide sequence of Enterococcus sp. isolate CC00264
SEQ ID NO:48-16s rRNA nucleotide sequence of Enterococcus sp. isolate CC00263
SEQ ID NO:49-16s rRNA nucleotide sequence of Enterococcus sp. isolate CC00619
SEQ ID NO:50-16s rRNA nucleotide sequence of Enterococcus sp. isolate CC00002
SEQ ID NO:51-16s rRNA nucleotide sequence of Enterococcus sp. isolate CC00066
SEQ ID NO:52-16s rRNA nucleotide sequence of Enterococcus sp. isolate mc071tn_zx_b3
SEQ ID NO:53-16s rRNA nucleotide sequence of Enterococcus sp. isolate mc071cn_fx_e6
SEQ ID NO:54-16s rRNA nucleotide sequence of Enterococcus sp. isolate mc071cn_fx_f8
SEQ ID NO:55-16s rRNA nucleotide sequence of Enterococcus sp. isolate mc001tny_c01
SEQ ID NO:56-16s rRNA nucleotide sequence of Enterococcus sp. isolate mc071tn_zx_d11
SEQ ID NO:57-16s rRNA nucleotide sequence of Enterococcus sp. isolate mc071tn_zx_d8
SEQ ID NO:58-16s rRNA nucleotide sequence of Enterococcus sp. isolate mc026tna2_b06
SEQ ID NO:59-16s rRNA nucleotide sequence of Enterococcus sp. isolate mc071tn_zx_c7
SEQ ID NO:60-16s rRNA nucleotide sequence of Enterococcus sp. isolate mc071cn_fx_h7
SEQ ID NO:61-16s rRNA nucleotide sequence of Enterococcus sp. isolate mc071tn_fx_c5
SEQ ID NO:62-16s rRNA nucleotide sequence of Enterococcus sp. isolate mc071tn_zx_b4
SEQ ID NO:63-16s rRNA nucleotide sequence of Enterococcus sp. isolate mc071tn_fx_d4
SEQ ID NO:64-16s rRNA nucleotide sequence of Enterococcus sp. isolate mc071tn_fx_d5
SEQ ID NO:65-16s rRNA nucleotide sequence of Enterococcus sp. isolate mc026cnm1_e07
SEQ ID NO:66-16s rRNA nucleotide sequence of Enterococcus sp. isolate mc071tn_fx_c10
SEQ ID NO:67-16s rRNA nucleotide sequence of Enterococcus sp. isolate mc071tn_fx_a9
SEQ ID NO:68-16s rRNA nucleotide sequence of Enterococcus sp. isolate mc071tn_fx_a4
SEQ ID NO:69-16s rRNA nucleotide sequence of Enterococcus sp. isolate mc071tn_zx_c2
SEQ ID NO:70-16s rRNA nucleotide sequence of Enterococcus sp. isolate mc071cn_zx_g1
SEQ ID NO:71-16s rRNA nucleotide sequence of Enterococcus sp. isolate mc071tn_zx_b7
SEQ ID NO:72-16s rRNA nucleotide sequence of Enterococcus sp. isolate mc071tn_fx_c1
SEQ ID NO:73-16s rRNA nucleotide sequence of Enterococcus sp. isolate mc026cna1_d06
SEQ ID NO:74-16s rRNA nucleotide sequence of Enterococcus sp. isolate mc071tn_fx_b1
SEQ ID NO's 75 to 88-Oligonucleotide primers
SEQ ID NO's 89 to 217-Further nucleotide sequences.

Where relevant an n in a sequence indicates the base was not resolved during the sequencing process, and this can be any base.

The sequences are listed below under the heading ‘Nucleotide Sequences’.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in biotherapeutics, prebiotics and the treatment of gastrointestinal tract dysbiosis and mucosal inflammation).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “bacteriotherapy” refers to the use of a bacterial isolate to treat or prevent a disease or a condition, or provide a health benefit, in a subject.

As used herein, the term “biotherapeutic” refers to a microorganism, such as bacterial isolate, that is useful for treating or preventing a disease or a condition, or provide a health benefit, in a subject.

The term “biotherapeutic composition”, as used herein, refers to a formulation comprising a biotherapeutic preparation formulated together with one or more additional formulary ingredients to obtain a finished formulation suitable for delivery to a subject.

As used herein, the “gastrointestinal tract” refers to the tract from the mouth to the anus which includes all the organs of the digestive system such as the esophagus, stomach, pancreas, liver, gallbladder, small intestine (including the ileum), caecum, large intestine, colon and rectum. Strains of the invention are at least useful for conditions of the terminal ileum, caecum or rectum.

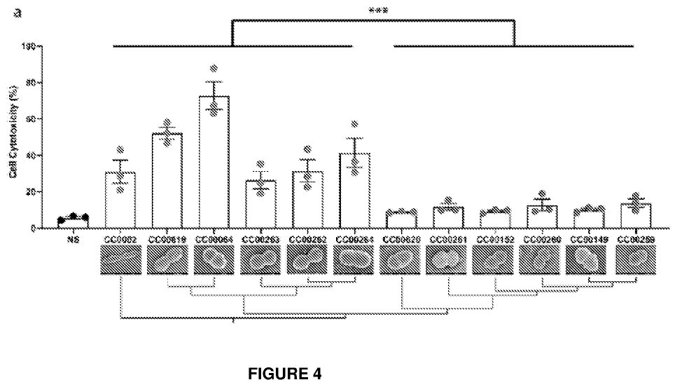

As used herein, a “non-inflammatory strain” refers to a strain of the invention which, when present in the gastrointestinal tract of a subject, preferably a human, is associated with a non-inflamed state. Non-inflammatory strains of the invention have little or no cytotoxicity against mammalian epithelial cells in culture, such as Caco2 cells as described in Example 2. In an embodiment, the strain results in less than 15%, less than 10% or less than 5% of cell death of the mammalian epithelial cells in culture. In an embodiment, a non-inflammatory strain of the invention causes less cell death when exposed to a given cell type than a strain which comprises a 16s ribosomal RNA (rRNA) gene having a nucleotide sequence as shown in any one of SEQ ID NO's 45 to 74, or a nucleotide sequence at least 90% identical to one or more of SEQ ID NO's 45 to 74, such as CC00064, CC00619, CC00262, CC0002 as described herein. In an embodiment, a non-inflammatory strain of the invention is a member of clade 149 as mentioned herein (see, for example, the clade in FIG. 4 covering strains CC00620, CC00261, CC00260, CC00149 and CC00259). Members of this clade have a 16s ribosomal RNA (rRNA) gene having a nucleotide sequence as shown in any one of SEQ ID NO's 1 to 44, or a nucleotide sequence at least 90% identical to one or more of SEQ ID NO's 1 to 44. In an embodiment, following 8 hours incubation with epithelial cells, such as Caco2 cells, one or more or all of the upregulated genes in Table 1 (designated as 1 in the last column) are upregulated by the non-inflammatory strain. In an embodiment, following 24 hours incubation with epithelial cells, such as Caco2 cells, one or more or all of the upregulated genes in Table 2 (designated as 1 in the last column) are upregulated by the non-inflammatory strain. In another embodiment, a non-inflammatory strain of the invention is detectable using primer pairs: CGAGGTACACCGTAAGTTGTTGCT (SEQ ID NO: 75) and ACCAGGACACGTAGACTTCACAGT (SEQ ID NO: 76), and/or ID NO: 77) TGGCCCAAAGATACGTGTTCCAC (SEQ and CGAACAAAATCAGGAGCTCGTCGT (SEQ ID NO: 78).

As used herein, an “inflammatory strain” refers to a strain of the invention which, when present in the gastrointestinal tract of a subject, preferably a human, is associated with an inflamed state. Inflammatory strains of the invention have cytotoxicity against mammalian epithelial cells in culture, such as Caco2 cells as described in Example 2. In an embodiment, the strain results at least 40%, at least 45% or at least 50% of cell death of the mammalian epithelial cells in culture. In an embodiment, an inflammatory strain of the invention causes more cell death when exposed to a given cell type than a strain which comprises a 16s ribosomal RNA (rRNA) gene having a nucleotide sequence as shown in any one of SEQ ID NO's 1 to 44, or a nucleotide sequence at least 90% identical to one or more of SEQ ID NO's 1 to 44, such as CC00620, CC00261, CC00260, CC00149 and CC00259 as described herein. In an embodiment, an inflammatory strain of the invention is a member of clade 64 as mentioned herein (see, for example, the clade in FIG. 4 covering strains CC00620, CC00261, CC00260, CC00149 and CC00259). Members of this clade have a 16s ribosomal RNA (rRNA) gene having a nucleotide sequence as shown in any one of SEQ ID NO's 45 to 74, or a nucleotide sequence at least 90% identical to one or more of SEQ ID NO's 45 to 74. In an embodiment, an inflammatory strain of the invention is a member of clade 64 as mentioned herein (see, for example, the clade in FIG. 4 covering strains CC00064, CC00619, CC00262, CC00263, and CC00264). Members of this clade have a 16s ribosomal RNA (rRNA) gene having a nucleotide sequence as shown in any one of SEQ ID NO's 45 to 74, or a nucleotide sequence at least 90% identical to one or more of SEQ ID NO's 45 to 74. In an embodiment, following 8 hours incubation with epithelial cells, such as Caco2 cells, one or more or all of the upregulated genes in Table 1 (designated as −1 in the last column) are upregulated by the inflammatory strain. In an embodiment, following 24 hours incubation with epithelial cells, such as Caco2 cells, one or more or all of the upregulated genes in Table 2 (designated as −1 in the last column) are upregulated by the inflammatory strain. In another embodiment, an inflammatory strain of the invention is detectable using primer pairs: ACTGCTGAGTGCCCACGCTT (SEQ ID NO: 79) and GAGGCACTTGCTAGCGTCACAC (SEQ ID NO: 80), TGCGTGTAGGCATCATCCGTGA (SEQ ID NO: 81) and GACACAGCGGCATCAGCAAGT (SEQ ID NO: 82), CCTTTACCATGGCCGTGTAGC (SEQ ID NO: 83) and CCATGAATGGTGTTCCTCCTTCTC (SEQ ID NO: 84), and/or AAACTTGCTGATGCCGCTGT (SEQ ID NO: 85) and CCGCCTTTACCGATAACCATACC (SEQ ID NO: 86).

The term “prebiotic”, as used herein, means an ingredient for inclusion in a biotherapeutic composition capable of inducing growth or activity of microorganisms in the gastrointestinal system.

As used herein, a “carrier” can be any solvents, diluents, excipients or other vehicles, dispersion or suspension aids, surface active agents, isotonic agents, thickening or emulsifying agents, preservatives, solid binders, lubricants and the like, as suited to the particular dosage form desired.

The term “subject” used within the context of the invention refers to a mammal including humans, livestock such horses, cows, sheep, goats and chickens, dogs and cats. In an embodiment, the subject is a human.

As used herein, the term “pharmaceutically acceptable carrier” component can refer to a component that is not biologically or otherwise undesirable, i.e., the component may be incorporated into a biotherapeutic composition of the invention and administered to a subject as described herein without causing any significant undesirable biological effects or interacting in a deleterious manner with any of the other components of the formulation in which it is contained. The component has generally met the required standards of toxicological and manufacturing.

As used herein, the terms “treat,” “treating,” “treatment” and grammatical variations thereof mean subjecting an individual subject to a protocol, regimen, process or remedy, in which it is desired to obtain a physiologic response or outcome in that subject. Since every treated subject may not respond to a particular treatment protocol, regimen, process or remedy, treating does not require that the desired physiologic response or outcome be achieved in each and every subject or subject population. Accordingly, a given subject or subject population may fail to respond or respond inadequately to treatment.

As used herein, the term “prevent”, “prevented”, or “preventing” when used with respect to the treatment of mucosal inflammation in the gastrointestinal refers to a prophylactic treatment which increases the resistance of a subject to mucosal inflammation in the gastrointestinal, in other words, decreases the likelihood that the subject will develop mucosal inflammation in the gastrointestinal as well as a treatment after mucosal inflammation in the gastrointestinal has begun in order to fight the inflammation, e.g., reduce or eliminate it altogether or prevent it from becoming worse.

As used herein, the term “reducing” or variations thereof refer to a reduction but not necessarily a complete abolition of gastrointestinal tract mucosal inflammation in a subject.

As used herein, the term “sample” refers to a collection of biological material obtained from a subject or a subject's surrounding environment, such as soil or water in the area that the subject inhabits. In some embodiments, the sample is obtained directly from the subject. For example, the sample can be a faecal sample or obtained during a colonoscopy. The sample may be in a form taken directly from the subject or surrounding environment, or it may be at least partially purified to remove at least some non-nucleic acid material. The purification may be slight, for instance amounting to no more than the concentration of the solids, or cells, of the sample into a smaller volume or the separation of cells from some or all of the remainder of the sample. In some embodiments, nucleic acids are isolated from the sample. Such isolated preparations include reverse transcription products and/or PCR amplification products of the nucleic acids in the sample. In some embodiments, the predominant nucleic acid is DNA. The nucleic acid preparations can be pure or partially purified nucleic acid preparations. Techniques for the isolation of nucleic acid from samples, including complex samples, are numerous and well known in the art.

The unit “cfu” refers to “colony forming unit”, which is the number of bacterial cells as revealed by microbiological counts on agar plates.

### Biotherapeutic Strains of Enterococcus sp.

The present invention provides numerous biotherapeutic strains of Enterococcus sp.

In an embodiment, a non-inflammatory strain of, or for use in, the invention comprises a 16s ribosomal RNA (rRNA) gene having a nucleotide sequence as shown in any one of SEQ ID NO's 1 to 44, or a nucleotide sequence at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8% or at least 99.9% identical to one or more of SEQ ID NO's 1 to 44.

In an embodiment, a non-inflammatory strain of, or for use in, the invention comprises a 16s ribosomal RNA (rRNA) gene having a nucleotide sequence as shown in any one of SEQ ID NO's 1 to 44, or a nucleotide sequence at least 94%, at least 94.5%, at least 95%, at least 95.5%, at least 96%, at least 96.5%, at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8% or at least 99.9% identical to one or more of SEQ ID NO's 1 to 44.

In an embodiment, a non-inflammatory strain of, or for use in, the invention comprises a 16s ribosomal RNA (rRNA) gene having a nucleotide sequence as shown in any one of SEQ ID NO's 1 to 44, or a nucleotide sequence at least 95% identical to one or more of SEQ ID NO's 1 to 44.

In an embodiment, a non-inflammatory strain of, or for use in, the invention comprises a 16s ribosomal RNA (rRNA) gene having a nucleotide sequence as shown in any one of SEQ ID NO's 1 to 44, or a nucleotide sequence at least 96% identical to one or more of SEQ ID NO's 1 to 44.

In an embodiment, a non-inflammatory strain of, or for use in, the invention comprises a 16s ribosomal RNA (rRNA) gene having a nucleotide sequence as shown in any one of SEQ ID NO's 1 to 44, or a nucleotide sequence at least 97% identical to one or more of SEQ ID NO's 1 to 44.

In an embodiment, a non-inflammatory strain of, or for use in, the invention comprises a 16s ribosomal RNA (rRNA) gene having a nucleotide sequence as shown in any one of SEQ ID NO's 1 to 44, or a nucleotide sequence at least 98% identical to one or more of SEQ ID NO's 1 to 44.

In an embodiment, a non-inflammatory strain of, or for use in, the invention comprises a 16s ribosomal RNA (rRNA) gene having a nucleotide sequence as shown in any one of SEQ ID NO's 1 to 44, or a nucleotide sequence at least 99% identical to one or more of SEQ ID NO's 1 to 44.

In an embodiment, a non-inflammatory strain of, or for use in, the invention comprises a 16s ribosomal RNA (rRNA) gene having a nucleotide sequence as shown in any one of SEQ ID NO's 1 to 44, or a nucleotide sequence at least 99.5% identical to one or more of SEQ ID NO's 1 to 44.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8% or at least 99.9% identical to one or more of SEQ ID NO's 45 to 74.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 94%, at least 94.5%, at least 95%, at least 95.5%, at least 96%, at least 96.5%, at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8% or at least 99.9% identical to one or more of SEQ ID NO's 45 to 74.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 95% identical to one or more of SEQ ID NO's 45 to 74.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 96% identical to one or more of SEQ ID NO's 45 to 74.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 97% identical to one or more of SEQ ID NO's 45 to 74.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 98% identical to one or more of SEQ ID NO's 45 to 74.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 99% identical to one or more of SEQ ID NO's 45 to 74.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 99.5% identical to one or more of SEQ ID NO's 45 to 74.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 97% identical to nucleic acid residues 1100 to 1210 of SEQ ID NO's 45 to 74.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 98% identical to nucleic acid residues 1100 to 1210 of SEQ ID NO's 45 to 74.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 99.5% identical to nucleic acid residues 1100 to 1210 of SEQ ID NO's 45 to 74.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 100% identical to nucleic acid residues 1100 to 1210 of SEQ ID NO's 45 to 74.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 100% identical to nucleic acid residues 1100 to 1210 of SEQ ID NO's 45 to 50.

In an embodiment, an inflammatory strain of, or for use in, the invention comprises a 16s ribosomal RNA (rRNA) gene having a nucleotide sequence as shown in any one of SEQ ID NO's 45 to 74, or a nucleotide sequence at least 100% identical to nucleic acid residues 1100 to 1210 of SEQ ID NO 45.

In an embodiment, an inflammatory strain of, or for use in, the invention comprises a 16s ribosomal RNA (rRNA) gene having a nucleotide sequence as shown in SEQ ID NO:45, or a nucleotide sequence at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8% or at least 99.9% identical to identical to SEQ ID NO:45.

In an embodiment, a strain of, or for use in, the invention comprises a 16s ribosomal RNA (rRNA) gene having a nucleotide sequence as shown in any one of SEQ ID NO's 1 to 74, or a nucleotide sequence at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8% or at least 99.9% identical to one or more of SEQ ID NO's 1 to 74.

In an embodiment, a strain of, or for use in, the invention comprises a 16s ribosomal RNA (rRNA) gene having a nucleotide sequence as shown in any one of SEQ ID NO's 1 to 74, or a nucleotide sequence at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8% or at least 99.9% identical to one or more of SEQ ID NO's 1 to 74, wherein the 16s ribosomal RNA (rRNA) gene does not comprise any one of SEQ ID NO's 89 to 217. Alternatively, the 16s ribosomal RNA (rRNA) gene does comprise any one of SEQ ID NO's 89 to 217.

In an embodiment, a strain of, or for use in, the invention comprises a 16s ribosomal RNA (rRNA) gene having a nucleotide sequence as shown in any one of SEQ ID NO's 1 to 44, or a nucleotide sequence at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8% or at least 99.9% identical to one or more of SEQ ID NO's 1 to 44, wherein the 16s ribosomal RNA (rRNA) gene does not comprise any one of SEQ ID NO's 89 to 217. Alternatively, the 16s ribosomal RNA (rRNA) gene does comprise any one of SEQ ID NO's 89 to 217.

In an embodiment, a strain of, or for use in, the invention comprises a 16s ribosomal RNA (rRNA) gene having a nucleotide sequence as shown in any one of SEQ ID NO's 45 to 74, or a nucleotide sequence at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8% or at least 99.9% identical to one or more of SEQ ID NO's 45 to 74, wherein the 16s ribosomal RNA (rRNA) gene does not comprise any one of SEQ ID NO's 89 to 217. Alternatively, the 16s ribosomal RNA (rRNA) gene does comprise any one of SEQ ID NO's 89 to 217.

In an embodiment, a strain of, or for use in, the invention comprises a 16s ribosomal RNA (rRNA) gene having a nucleotide sequence as shown in any one of SEQ ID NO's 45 to 50, or a nucleotide sequence at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8% or at least 99.9% identical to one or more of SEQ ID NO's 45 to 50, wherein the 16s ribosomal RNA (rRNA) gene does not comprise any one of SEQ ID NO's 89 to 217. Alternatively, the 16s ribosomal RNA (rRNA) gene does comprise any one of SEQ ID NO's 89 to 217.

In an embodiment, a strain of, or for use in, the invention comprises a 16s ribosomal RNA (rRNA) gene having a nucleotide sequence of SEQ ID NO 45, or a nucleotide sequence at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO 45, wherein the 16s ribosomal RNA (rRNA) gene does not comprise any one of SEQ ID NO's 89 to 217. Alternatively, the 16s ribosomal RNA (rRNA) gene does comprise any one of SEQ ID NO's 89 to 217.

In an embodiment, the % identity of a polynucleotide is determined by GAP (Needleman and Wunsch, 1970) analysis (GCG program) with a gap creation penalty=5, and a gap extension penalty=0.3. Preferably, the GAP analysis aligns two sequences over their entire length.

The bacterial strains for use in the present invention can be cultured using standard microbiology techniques as detailed in, for instance, Handbook of Microbiological Media, Fourth Edition (2010) Ronald Atlas, CRC Press, Maintaining Cultures for Biotechnology and Industry (1996) Jennie C. Hunter-Cevera, Academic Press, as well as how detailed in the Examples using YCFA medium.

### Methods Treatment or Prevention

Methods of the invention can be used to treat or prevent a dysbiosis of the gastrointestinal tract in a subject. “Dysbiosis” in the context of the present invention refers to a state in which the normal diversity and/or function of the microbiota or microbiome, in particular the human gastrointestinal microbiota, is disrupted. Any disruption from the normal state of the microbiota in a healthy individual can be considered a dysbiosis, even if the dysbiosis does not result in a detectable decrease in health in the individual. In a preferred embodiment, the dysbiosis may be associated with one or more pathological symptoms. For example, “dysbiosis” may refer to a decrease in the microbial diversity of the microbiota. In addition, or alternatively, “dysbiosis” may refer to an increase in the abundance of one or more bacteria, e.g. one or more pathogenic bacteria, in the microbiota of an individual relative to the abundance of said bacterium or bacteria in the microbiota of a healthy individual, i.e. an individual without a dysbiosis. The pathogenic bacteria present during dysbiosis are often Proteobacteria and resistant to one or more antibiotics. Examples of Proteobacteria include Escherichia, Salmonella, Campylobacter, Vibrio, Helicobacter, and Yersinia species.

The dysbiosis may be a dysbiosis associated with an enteric bacterial infection, such as an infection of the gastrointestinal tract with a pathogenic bacterium. Many bacteria capable of causing infections of the gastrointestinal tract in humans are known and include: gram positive bacteria, and gram negative bacteria. The pathogenic bacterium is preferably a pathogenic species of the genus Clostridium, Escherichia, Enterococcus, Klebsiella, Enterobacter, Proteus, Salmonella, Shigella, Staphylococcus, Vibrio, Aeromonas, Campylobacter, Plesiomonas, Bacillus, Helicobacter, Listeria, or Yersinia. Preferred examples of such pathogenic bacteria include Clostridium difficile, Clostridium perfringens, Clostridium botulinum, Escherichia coli, Salmonella typhi, Staphylococcus aureus, Vibrio cholerae, Vibrio parahaemolyticus, Vibrio vulnificus, Campylobacter fetus, Campylobacter jejuni, Aeromonas hydrophila, Plesiomonas shigelloides, Bacillus cereus, Helicobacter pylori, Listeria monocytogenes, and Yersinia enterocolitica. More preferably, the pathogenic bacterium is a pathogenic species of the genus Clostridium or Escherichia. Most preferably, the pathogenic bacterium is Clostridium difficile or Escherichia coli.

Methods of the invention can be used to reduce or prevent gastrointestinal disorders.

Methods of the invention can be used to reduce or prevent gastrointestinal tract mucosal inflammation in a subject using a non-inflammatory (non-cell cytotoxic) strain of Enterococcus sp. of the invention.

In an embodiment, the subject has, or is susceptible to having, an inflammatory bowel diseases (IBD) such as Crohn's disease, ulcerative colitis or pouchitis. As used herein, the term “inflammatory bowel diseases (IBD)” has its general meaning in the art and refers to a group of inflammatory diseases of the colon and small intestine such as revised in the World Health Organisation Classification K20-K93 (ICD-10) such as Crohn disease (such as granulomatous enteritis; Crohn disease of small intestine; Crohn disease of large intestine; granulomatous and regional Colitis; Crohn disease of colon, large bowel and rectum; Crohn disease of both small and large intestine), Ulcerative colitis (such as Ulcerative (chronic) pancolitis; backwash ileitis; Ulcerative (chronic) proctitis; Ulcerative (chronic) rectosigmoiditis; Inflammatory polyps; Left sided colitis; left hemicolitis) and noninfective gastroenteritis and colitis (Gastroenteritis and colitis due to radiation; Toxic gastroenteritis and colitis; Allergic and dietetic gastroenteritis and colitis; Food hypersensitivity gastroenteritis or colitis; indeterminate colitis; specified noninfective gastroenteritis and colitis such as Collagenous colitis; Eosinophilic gastritis or gastroenteritis; Lymphocytic colitis Microscopic colitis (collagenous colitis or lymphocytic colitis); Noninfective gastroenteritis and colitis such as Diarrhoea; Enteritis; Ileitis; Jejunitis; Sigmoiditis) and postprocedural disorders of digestive system such as pouchitis. In an embodiment, the IBD is paediatric IBD.

In a further aspect, the present invention also relates to a fecal microbiota transplant composition comprising the strain of the invention. The term “fecal microbiota transplant composition” has its general meaning in the art and refers to any composition that can restore the fecal microbiota.

In a further aspect, there is a method of decreasing the growth of pathogenic bacteria, said method comprising the step of administering to a patient in need thereof, a composition comprising a non-inflammatory (non-cell cytotoxic) strain of Enterococcus sp. of the invention. In a preferred embodiment, the method reduces the relative abundance of pathogenic bacterium or decolonises pathogenic bacterium.

In a further aspect, there is a method of increasing the growth of healthy bacteria, said method comprising the step of administering to a patient in need thereof, a composition comprising a non-inflammatory (non-cell cytotoxic) strain of Enterococcus sp. of the invention.

Administration to humans includes administration by a medical professional and self-administration. In general, in order to achieve a health benefit, multiple doses of the biotherapeutic composition are administered, for example daily for a period of at least one week, at least two weeks, at least three weeks, at least six weeks, at least nine weeks, or at least twelve weeks. In one embodiment, the biotherapeutics can be administered for the remaining duration of a subject's life.

In a further embodiment, the composition is administered to the patient using a dosing regimen selected from the group consisting of: hourly; every 2 hours; every 3 hours; every 4 hours; every 5 hours; every 6 hours; every 12 hours; once daily; twice daily; every 2 days; every 3 days; every 4 days; every 5 days; every 6 days; weekly; twice weekly; every 2 weeks; every 3 weeks; every 4 weeks; every 5 weeks; every 6 weeks; once monthly; twice monthly; every 2 months; every 3 months; every 4 months; every 5 months; every 6 months; yearly; twice yearly; every 2 years; every 3 years; every 4 years; and every 5 years.

In an embodiment, the method reduces the relative abundance of a member of the bacteria genus or decolonises a member of the bacteria genus. Preferably, the method drives down undesired inflammation. Preferably, the said method decreases inflammation in the subject when measured by a parameter selected from the group consisting of: TNFα signalling via NF-κB; IFNα signalling; IFNγ signalling; IL6 JAK STAT3 signalling; activation of pro-apoptotic pathways; initiation of unfolded protein response. Preferably, the said method upregulates genes associated with pro-apoptotic pathways and the unfolded protein response, including genes selected from the group consisting of: CHAC1, CEBPB, TRIB3, PPP1R15A, DDIT3, ATF4 and XBP1.

Preferably, the composition is administered orally or rectally.

### Compositions

The therapeutic composition of the invention may comprise a pharmaceutically acceptable excipient, carrier, buffer, stabilizer or other materials well known to those skilled in the art. Such materials should be non-toxic and should not interfere with the efficacy of the isolated bacteria present in the therapeutic composition. The precise nature of the pharmaceutically acceptable excipient or other material will depend on the route of administration, which may be, for example, oral or rectal. Many methods for the preparation of therapeutic compositions are known to those skilled in the art (see e.g. Robinson ed., Sustained and Controlled Release Drug Delivery Systems, Marcel Dekker, Inc., New York, 1978).

The therapeutic composition of the invention may comprise a prebiotic, a carrier, insoluble fibre, a buffer, an osmotic agent, an anti-foaming agent and/or a preservative.

The therapeutic composition may be made or provided in chemostat medium. Alternatively, the therapeutic composition may be made or provided in saline, e.g., 0.9% saline. It will be understood that any carrier or solution which does not impair viability of the bacteria present in the therapeutic composition and is compatible with administration to an individual may be used.

The therapeutic composition may be made or provided under reduced atmosphere, i.e., in the absence of oxygen. A synthetic stool preparation may be made or provided under N₂, CO₂, H₂, or a mixture thereof, optionally with controlled levels of partial pressure of N₂: CO₂: H₂.

The therapeutic composition may be for oral or rectal administration to the individual. Where the therapeutic composition is for oral administration, the therapeutic composition may be in the form of a capsule, or a tablet. Where the therapeutic composition is for rectal administration, the therapeutic composition may be in the form of an enema. The preparation of suitable capsules, tablets and enema is well-known in the art. The capsule or tablet may comprise a coating to protect the capsule or tablet from stomach acid. For example, the capsule or tablet may be enteric-coated, pH dependant, slow-release, and/or gastro-resistant. Such capsules and tablets are used, for example, to minimize dissolution of the capsule or tablet in the stomach but allow dissolution in the small intestine.

Orally dosed formulations, for example, can, in addition to the viable microorganisms comprise, inert compression aids, such as microcrystalline cellulose or oligosaccharide, flow aids, such as a silica gel, or a lubricant of, for example magnesium stearate (vegetable source) or stearic acid (vegetable source).

A composition disclosed herein can be used as, for example, a food supplement, an edible product or pharmaceutical product. When it is a food supplement, the biotherapeutic composition can further comprise a conventional food supplement filler and/or an extender. The biotherapeutic composition disclosed herein can also be included in any edible products, such as dairy products, including for example, a milk product, milk, yogurt, curd, ice-cream, dressing, and cheese, beverage products, meat products, and baked goods

Suppository formulations, for example, either for rectal use, can in addition to the biotherapeutics, comprise, for example, cocoa butter, polyethylene glycol, glycerine or gelatine.

The composition may comprise a disintegrant, a glidant, and/or a lubricant. Disintegrants aid in the breakup of the compacted mass when placed in a fluid environment. The disintegrant may be any suitable disintegrant such as for example, a disintegrant selected from the group consisting of sodium croscarmellose, crospovidone, gellan gum, hydroxypropyl cellulose, starch, and sodium starch glycolate. The glidant may be any suitable glidant such as for example, a glidant selected from the group consisting of silicon dioxide, colloidal silicon dioxide, and talc. Lubricants are generally always used in the manufacture of dosage forms by direct compression in order to prevent the compacted powder mass from sticking to the equipment during the tabletting or encapsulation process. The lubricant may be any suitable lubricant such as for example, a lubricant selected from the group consisting of calcium stearate, magnesium stearate, stearic acid, sodium stearyl fumerate, and vegetable based fatty acids. In the composition and method of the present invention, the carrier, may be present in the composition in a range of approximately 30% w/w to approximately 98% w/w; this weight percentage is a cumulative weight percentage taking into consideration all ingredients present in the carrier.

Coatings can be used to control the solubility of the composition. Examples of coatings include carrageenan, cellulose acetate phthalate, ethylcelulose, gellan gum, matodextrin, methacrylates, methylcellulose, microcrystalline cellulose, and shellac.

The composition may comprise one or more preservatives. Exemplary preservatives include antioxidants, chelating agents, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabi sulfite, propionic acid, propyl gallate, sodium ascorbate, sodium bisulfite, sodium metabi sulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabi sulfite, potassium sulfite, potassium metabi sulfite, Glydant Plus, Phenonip, methylparaben, Germall 115, Germaben II, Neolone, Kathon, and Euxyl. I.

The therapeutic composition may be a liquid.
The therapeutic composition may be lyophilized. The lyophilized therapeutic composition may comprise one or more stabilisers and/or cryoprotectants. The lyophilized therapeutic composition may be reconstituted using a suitable diluent prior to administration to the individual. Preferably, the cryoprotectant is selected from the group consisting of: trehalose; mannitol; sucrose; glycerol; sorbitol; DMSO; propylene glycol; ethylene glycol; saccharose; galactose-lactose; inulin; maltodextrin; and any combination thereof. Preferably, the said cryoprotectant further comprises a compound selected from the group consisting of: glycerol; polyethylene glycol (PEG); glycerin; erythritol; arabitol; xylitol; sorbitol; glucose; lactose; ribose; and any combination thereof. Preferably, the said cryoprotectant is trehalose at a concentration of 2% to 15% in said lyophilized formulation. Preferably, the said cryoprotectant is trehalose at a concentration of at least 5% in said lyophilized formulation. Preferably, the said cryoprotectant is trehalose at a concentration of at least 10% in said lyophilized formulation.
The composition of any one of claims X to Y, wherein the at least one strain of bacteria is diluted with an inert powdered diluent.

In an embodiment, after at least 4 weeks of storage at room temperature, said composition is capable of maintaining at least 10% cell viability relative to the initial cell viability immediately prior to storage and pre-lyophilisation.

In an embodiment, after at least 4 weeks of storage at room temperature, said composition is capable of maintaining at least 50% cell viability relative to the initial cell viability immediately prior to storage and post-lyophilisation.

In an embodiment, after at least 4 weeks of storage at room temperature said composition is capable of maintaining about 60% to about 80% cell viability relative to the initial cell viability immediately prior to the start of said storage.

In an embodiment, said composition decreases inflammation in the subject when measured by a parameter selected from the group consisting of: TNFα signalling via NF-κB; IFNα signalling; IFNγ signalling; IL6 JAK STAT3 signalling; activation of pro-apoptotic pathways; initiation of unfolded protein response.

In an embodiment, said composition down regulates genes associated with pro-apoptotic pathways and the unfolded protein response, including genes selected from the group consisting of: CHAC1, CEBPB, TRIB3, PPP1R15A, DDIT3, ATF4 and XBP1.

In an embodiment, the bacteria is cultured from a faecal or colonic biopsy sample. In an embodiment, the bacteria comprises a community of bacterial cells derived from a stool or biopsy of one or more human donors. In an embodiment, the community of bacterial cells comprises cultured bacterial cells. In an embodiment, the cultured bacterial cells are derived from a multiple of human donors. In an embodiment, the community of bacterial cells comprises uncultured bacterial cells. In an embodiment, the uncultured bacterial cells are derived from a single human donor. In an embodiment, the composition is a faecal transplant microbiota composition.

A therapeutic composition according to the present invention may be administered alone or in combination with other treatments, concurrently or sequentially or as a combined preparation with another therapeutic agent or agents, for the treatment of dysbiosis, or a disease associated with dysbiosis as described herein. For example, a strain of the invention may be used in combination with an existing therapeutic agent for inflammatory bowel disease, irritable bowel syndrome, a metabolic disease, a neuropsychiatric disorder, an autoimmune disease, an allergic disorder, a cancer, or hepatic encephalopathy.

For example, where the therapeutic composition is for the treatment of a dysbiosis associated with cancer, the therapeutic composition may optionally be administered in combination with a cancer immunotherapy, such as an immune check-point inhibitor, to the individual. Examples of check-point inhibitors which may be employed in this context include Programmed cell death protein 1 (PD-1) inhibitors, Programmed death-ligand 1 (PD-L1) inhibitors, cytotoxic T-lymphocyte-associated protein 4 (CTLA-4) inhibitors. Manipulation of the gut microbiota in combination with immune check-point inhibitor treatment has been shown to improve efficacy of immune check-point inhibitors in treating cancer. In a preferred embodiment, the cancer in this context is lung cancer or melanoma.

In another embodiment, the composition of the invention further comprise immunomodulating compounds. In other embodiments, the immunomodulating compound is a cytokine, chemokine, or complement component that enhances expression of immune system accessory or adhesion molecules, their receptors, or combinations thereof. In some embodiments, the immunomodulating compound include interleukins, for example interleukins 1 to 15, interferons alpha, beta or gamma, tumour necrosis factor, granulocyte-macrophage colony stimulating factor (GM-CSF), macrophage colony stimulating factor (M-CSF), granulocyte colony stimulating factor (G-CSF), chemokines such as neutrophil activating protein (NAP), macrophage chemoattractant and activating factor (MCAF), RANTES, macrophage inflammatory peptides MIP-1a and MIP-1b, complement components, or combinations thereof. In other embodiments, the immunomodulating compound stimulate expression, or enhanced expression of OX40, OX40L (gp34), lymphotactin, CD40, CD40L, B7.1, B7.2, TRAP, ICAM-1, 2 or 3, cytokine receptors, or combination thereof.

In another embodiment, the immunomodulatory compound induces or enhances expression of co-stimulatory molecules that participate in the immune response, which include, in some embodiments, CD40 or its ligand, CD28, CTLA-4 or a B7 molecule. In another embodiment, the immunomodulatory compound induces or enhances expression of a heat stable antigen (HSA), chondroitin sulfate-modified MHC invariant chain (li-CS), or an intracellular adhesion molecule 1 (ICAM-1).

The therapeutic compositions of the invention may be administered to an individual, preferably a human individual. Administration may be in a “therapeutically effective amount”, this being sufficient to show benefit to the individual. Such benefit may be at least amelioration of at least one symptom. Thus “treatment” of a specified disease refers to amelioration of at least one symptom. The actual amount administered, and rate and time-course of administration, will depend on the nature and severity of what is being treated, the particular patient being treated, the clinical condition of the individual patient, the cause of the dysbiosis, the site of delivery of the composition, the type of therapeutic composition, the method of administration, the scheduling of administration and other factors known to medical practitioners. Prescription of treatment, e.g. decisions on dosage etc., is within the responsibility of general practitioners and other medical doctors, and may depend on the severity of the symptoms and/or progression of a disease being treated. A therapeutically effective amount or suitable dose of a therapeutic composition of the invention can be determined by comparing its in vitro activity and in vivo activity in an animal model. Methods for extrapolation of effective dosages in mice and other test animals to humans are known. The precise dose will depend upon a number of factors, including whether the therapeutic composition is for prevention or for treatment.

Formulary ingredients can be contacted with a biotherapeutic preparation and mixed or prepared until a biotherapeutic formulation is obtained. As will be clear to those of skill in the art, formulation conditions will generally be such that viable microorganisms are retained. In particular high temperatures, for example temperatures in excess of 40° C. are avoided.

The amount of viable microorganisms included in a biotherapeutic composition can vary and can be adjusted and optimized as will be appreciated by those of skill in the art Such optimization may, for example, be achieved by preparing a series of different doses of a viable microorganism. The bacterial concentration in the composition can be, for example, from 10 million cfu/mL to 100 billion cfu/mL, from 10 million to 50 million cfu/mL, more preferably from 50 million to 100 million cfu/mL, from 100 million to 500 million cfu/mL, from 500 million to 1 billion cfu/mL, from 1 billion to 5 billion cfu/mL, from 5 billion to 10 billion cfu/mL, from 10 billion to 15 billion cfu/mL, from 15 billion to 20 billion cfu/mL, from 20 billion to 25 billion cfu/mL, from 25 billion to 30 billion cfu/mL, from 30 billion to 35 billion cfu/mL, from 35 billion to 40 billion cfu/mL, from 40 billion to 45 billion cfu/mL, from 45 billion to 50 billion cfu/mL, from 50 billion to 55 billion cfu/mL, from 55 billion to 60 billion cfu/mL, from 60 billion to 65 billion cfu/mL, from 65 billion to 70 billion cfu/mL, from 70 billion to 75 billion cfu/mL, from 75 billion to 80 billion cfu/mL, from 80 billion to 85 billion cfu/mL, from 85 billion to 90 billion cfu/mL, from 90 billion to 95 billion cfu/mL, from 95 billion to 100 billion cfu/mL.

In an embodiment, the strain of the invention can be administered at, for example, a dosage of 0.01 to 100×10¹¹ cells/body, 0.1 to 10×10¹¹ cells/body or 0.3 to 5×10¹¹ cells/body. Furthermore, for example, the amount ingested per day as the bacteria can be 0.01 to 100×10¹¹ cells/60 kg body weight, 0.1 to 10×10¹¹ cells/60 kg body weight or 0.3 to 5×10¹¹ cells/60 kg body weight.

The content of the biotherapeutic contained in the orally ingested composition of the present invention may be determined as appropriate depending on its application form. As biotherapeutic dry microbial body it can be, for example, 5 to 50 w/w %, 1 to 75 w/w %, 0.1 to 100 w/w % or 1 to 100 w/w %.

In an embodiment, every 200 mg of the composition comprises a pharmacologically active dose of bacteria cells or spores selected from the group consisting of: 10³ to 10¹⁴; 10⁴ to 10¹⁴; 10⁵ to 10¹⁴; 10⁶ to 10¹⁴; 10⁷ to 10¹⁴; 10⁸ to 10¹⁴; 10⁴ to 10¹³; 10⁵ to 10¹²; 10⁶ to 10¹¹; 10⁷ to 10¹⁰; 10⁸ to 10⁹; 10³ to 10¹³; 10³ to 10¹²; 10³ to 10¹¹; 10³ to 10¹⁰; 10³ to 10⁹; 10³ to 10⁸; 10³ to 10⁷; 10³ to 10⁶; 10³ to 10⁵, and 10³ to 10⁴ colony forming units (cfu) or total cell count.

In an embodiment, the composition comprises a pharmacologically active dose of bacteria cells or spores selected from the group consisting of: from 10 million cfu/mL to 100 billion cfu/mL, from 10 million to 50 million cfu/mL, more preferably from 50 million to 100 million cfu/mL, from 100 million to 500 million cfu/mL, from 500 million to 1 billion cfu/mL, from 1 billion to 5 billion cfu/mL, from 5 billion to 10 billion cfu/mL, from 10 billion to 15 billion cfu/mL, from 15 billion to 20 billion cfu/mL, from 20 billion to 25 billion cfu/mL, from 25 billion to 30 billion cfu/mL, from 30 billion to 35 billion cfu/mL, from 35 billion to 40 billion cfu/mL, from 40 billion to 45 billion cfu/mL, from 45 billion to 50 billion cfu/mL, from 50 billion to 55 billion cfu/mL, from 55 billion to 60 billion cfu/mL, from 60 billion to 65 billion cfu/mL, from 65 billion to 70 billion cfu/mL, from 70 billion to 75 billion cfu/mL, from 75 billion to 80 billion cfu/mL, from 80 billion to 85 billion cfu/mL, from 85 billion to 90 billion cfu/mL, from 90 billion to 95 billion cfu/mL, from 95 billion to 100 billion cfu/mL.

In an embodiment, the composition comprises a pharmacologically active dose of bacteria cells or spores wherein the concentration of the bacteria cells or spores as a dry microbial body, is selected from the group consisting of: between 5 to 50 w/w %, 1 to 75 w/w %, 0.1 to 100 w/w % and 1 to 100 w/w %.

In an embodiment, the composition is a controlled release composition. As used herein, the term “controlled-release” refers to release or administration of a strain of the invention from a given dosage form in a controlled fashion in order to achieve the desired pharmacokinetic profile in vivo. An aspect of “controlled” delivery is the ability to manipulate the formulation and/or dosage form in order to establish the desired kinetics of biotherapeutic release.

Procedures for preparing tablets, caplets, capsules and other forms of compositions of the invention are known to those of ordinary skill in the art and include without limitation wet granulation, dry granulation, and direct compression (for tablets and caplets).

Wet and dry granulation is used to manufacture tablets, caplets, or capsules. With granulation techniques, a chilsonation is used to manufacture the powder for the dosage forms. A chilsonator houses grooved, rotating rollers that are pressed tightly against one another by hydraulic pressure. Raw materials are placed into the hopper of the chilsonator and are fed by a system of horizontal and vertical screws into the rollers. As materials pass through the grooves in the rollers, it is compacted under very high pressure and emerges from the chilsonator as dense sheets. The sheets are milled into a fine granular powder using a Fitz mill and then passed through a screen to produce a uniform free flowing granule. The chilsonation process results in a finished powder that is two to four times denser than the starting material, a feature that permits the ingredients to be fashioned into the desired dosage form.

With dry granulation, the powder may be incorporated into a gelatin capsule or it may be mixed with gelatin to form a tablet or caplet. With wet granulation, the powder is moistened thus creating large “chunks” of material that are subsequently dried and milled to convert the chunks to particles of a desired size for the manufacturing process. Once the particles of a desired size are obtained, the particles are incorporated into a gelatin capsule or mixed with gelatin to form a tablet or caplet.

General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

In a further aspect, there is a method of preparing a composition of this invention, said method comprising culturing at least one biotherapeutic strain of Enterococcus sp and mixing the at least one strain with a pharmaceutically acceptable carrier.

### Prebiotics

A composition of the invention can comprise a prebiotic. Because prebiotics have a chemical structure that resists digestion through the alimentary tract, they reach the colon as intact molecules where they are able to elicit systemic physiological functions and act as fermentable substrates for colonic microflora. Where a prebiotic is combined with a biotherapeutic, the resulting composition is sometimes referred to as a “synbiotic.”

Examples of suitable prebiotics include, but are not limited to, oligosaccharide such as fructooligosaccharides, P95 Nutraflora®, for example, galactooligosaccharides, xylooligosaccharides, isomaltooligosaccharides, human milk oligosaccharides, inulin oligosaccharides, mannan oligosaccharides, pyrodextrin, levan, maltotriose, pectic oligosaccharides, bimuno-galactooligosaccharides, arabinoxylan, and fucoidan. Fructooligosaccharides can be extracted from, for example, chicory, artichokes, asparagus, dandelions, dahlias, endive, garlic, leeks, lettuce, and onions.

In an embodiment, the prebiotic comprises amino acids such as one or more or all of alanine, aspartic acid, glutamic acid, glycine, leucine, isoleucine, proline, serine, threonine and valine.

In an embodiment, the prebiotic comprises simple sugars which can be a monosaccharide (such as glucose, galactose or fructose) and/or a disaccharide (such as sucrose maltose or lactose).

In an embodiment, the prebiotic comprises from about 5% (w/w) to about 50% (w/w), about 7.5% (w/w) to about 30% (w/w) or about 10% (w/w) to about 15% (w/w) of the composition.

### Other Microorganisms

In order to obtain the desired health benefit to the subject, it may be advantageous to include one or more additional biotherapeutic microorganisms in the composition. Thus, the composition may comprise more than one species/strain of microorganisms in addition to the strain of the invention, such as two, three, four, five or a higher plurality of species/strains of microorganisms. Non-limiting examples of biotherapeutics are suitable strains of the genus Aerococcus, Adlercreutzia, Allobaculum, Bacillus, Bifidobacterium, Carnobacterium, Clostridium, Eubacterium, Enterococcus (in addition to an Enterococcus strain of the invention), Oenococcus, Lactobacillus, Lactococcus, Leuconostoc, Pediococcus, Propionibacterium, Sporolactobacillus, Staphylococcus, Streptococcus and Tetragenococcus, Vagococcus and Weisella. It is to be understood that the foregoing list is intended only to be illustrative and not a limiting representation of the biotherapeutics that may be included in the composition of the present invention. In this respect, any additional biotherapeutic species may also be used in the compositions of the present invention.

In an embodiment, the Enterococcus sp. is Enterococcus faecalis or Enterococcus faecium.

In an embodiment, the Lactobacillus sp. is selected from the group consisting of Lactobacillus rhamnosus (such as strain GG (ATCC 53103), CGMCC 1.3724 or SP1 (DSM 21690)), Lactococcus lactis, Lactococcus cremoris, Lactococcus diacetylactis, Lactobacillus paracasei, Lactobacillus reuteri (such as strain ATCC 55730 or DSM 17938), Lactobacillus acidophilus, Lactobacillus murinus, Lactobacillus helveticus, Lactobacillus bulgaricus, Lactobacillus casei, Lactobacillus salivarius, Lactobacillus plantarum, Lactobacillus fermentum, Lactobacillus taiwanensis, Lactobacillus animalis, Lactobacillus johnsonii (such as strain NCC533; CNCM 1-1225) and Lactobacillus gasseri.

In an embodiment, the Bifidobacterium sp. is selected from the group consisting of Bifidobacterium lactis (such as strain BB-12, BI-04 or CNCM 1-3446 (Bb12)), Bifidobacterium longum (such as strain NCC3001, ATCC BAA-999 (BB536)), Bifidobacterium breve (such as strain Bb-03, M-16V or R0070), Bifidobacterium infantis, Bifidobacterium animalis, Bifidobacterium bifidum and Bifidobacterium adolescentis.

In an embodiment, the Streptococcus sp. is Streptococcus thermophilus such as Streptococcus thermophilus ST-21

In an embodiment, the Clostridium sp. is Clostridium difficile.

Some yeasts are also useful as biotherapeutics and are sometimes included in the biotherapeutic compositions. One non-limiting example of a yeast used in biotherapeutics is Saccharomyces boulardii.

### Dosage Forms and Kits

In a further aspect of the invention, there is a dosage form comprising the composition according to an earlier aspect of the invention.

In a further aspect of the invention, there a kit comprising the dosage form together with instructions for its use.

### Uses

In a further aspect of the invention, there is the use of the composition according to an earlier aspect of the invention in the manufacture of a medicament for reducing or preventing a gastrointestinal disorder in a subject.

### Methods of Detection

A strain of the invention can be detected using a wide variety of known techniques. Conveniently the strain is detected using a nucleic acid based detection system.

In an embodiment, nucleic acid sequencing is used. Illustrative non-limiting examples of nucleic acid sequencing techniques include, but are not limited to, chain terminator (Sanger) sequencing and dye terminator sequencing. In some embodiments, the technology provided herein finds use in a Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology.

In some embodiments, hybridization is employed in a detection method of the invention. Illustrative non-limiting examples of nucleic acid hybridization techniques include, but are not limited to, in situ hybridization (ISH), microarray, and Southern or Northern blot. In one embodiment, a FISH assay is used.

In other embodiments, nucleic acid amplification is used. Nucleic acids may be amplified prior to or simultaneous with detection. Conducting one or more amplification reactions may comprise one or more PCR-based amplifications, non-PCR based amplifications, or a combination thereof. Illustrative non-limiting examples of nucleic acid amplification techniques include, but are not limited to, polymerase chain reaction (PCR), reverse transcription polymerase chain reaction (RT-PCR), nested PCR, linear amplification, multiple displacement amplification (MDA), real-time SDA, rolling circle amplification, circle-to-circle amplification transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

Non-amplified or amplified nucleic acids can be detected by any conventional means. For example, the nucleic acids can be detected by hybridization with a detectably labeled probe and measurement of the resulting hybrids. In another example, the nucleic acids are detected by sequencing. Illustrative non-limiting examples of detection methods are described herein.

Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205. Another method for determining the quantity of target sequence initially present in a sample, but which is not based on a real-time amplification, is disclosed in U.S. Pat. No. 5,710,029.

Amplification products may be detected in real-time through the use of various self-hybridizing probes, most of which have a stem-loop structure. Such self-hybridizing probes are labelled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097.

In an embodiment, the method includes quantifying the amount of strain present in the sample.

In a further aspect, there is a method of analysing the microbiome of the gastrointestinal tract of a subject, the method comprising; obtaining a sample comprising bacteria from the gastrointestinal tract of the subject, analysing the sample for the presence of at least one strain of a biotherapeutic strains of Enterococcus sp.

In a further aspect, there is a method of analysing the microbiome of the gastrointestinal tract of a subject, the method comprising; obtaining a sample comprising bacteria from the gastrointestinal tract of the subject, analysing the sample for the presence of at least one strain of a pathogenic bacteria.

### EXAMPLES

### Example 1—Materials and Methods

### Sample Collection

Fresh mucosal sample were obtained during paediatric endoscopy lists, from consenting patients receiving clinically indicated colonoscopies. Six mucosal samples were obtained from each patient, with paired samples collected from three intestinal regions (Terminal lleum, Caecum, Rectum), to allow for matched mucosal culturing and transcriptional profiling. The samples for bacterial culturing were placed into anaerobic conditions within 15 minutes of collection. The samples for RNA sequencing were collected in 200 μl of RNAlater stabilisation solution (QIAGEN; Hilden, Germany) and stored at −80° C.

### Bacterial Culturing

Sample processing took place under anaerobic conditions in a Whitley A95 workstation (Don Whitley Scientific; Yorkshire, United Kingdom) at 37° C. All reagents needed for bacterial culturing were pre-reduced in the anaerobic environment 24 hours prior to sample processing. To process, the samples were weighed, diluted by a factor of 10 with sterile pre-reduced PBS, serially diluted down to 10⁻⁶ and plated directly onto YCFA agar (see WO 2021/163758). Samples were then grown in anaerobic, microaerophilic and aerobic conditions. The plates for culturing of anaerobic bacteria were incubated at 37° C. in the Whitely A95 anaerobic workstation (Don Whitley Scientific; Yorkshire, United Kingdom), containing 10% carbon dioxide, 10% hydrogen and 80% nitrogen. The plates for culturing of aerobic bacteria were incubated at 37° C. in the Bio concept incubator (Froilabo; Lionel Terray, Meyzieu, France). The plates for culturing of microaerophilic bacteria were stored in 2.5 L gas jars (Thermo Scientific; Waltham, Massachusetts, United States) containing 2.5 L CampyGen gas packs (Oxoid; Basingstoke, Hampshire, United Kingdom), and incubated at 37° C. in the Bio concept incubator (Froilabo). Bacterial colony picking was performed 24 hours after plating, using plates harbouring distinct, non-converging bacterial colonies. To culture for metagenomic analysis, 50 μl aliquots of each dilution factor were applied to YCFA agar plates and uniformly spread across the plate using a disposable plate spreader (International Scientific Group). The plates were incubated at 37° C., in the appropriate environment. The plates were scraped for metagenomic analysis 24 hours after plating, using plates harbouring distinct, non-converging bacterial colonies.

### Bacterial Purification and Archiving

Isolates were restreaked onto YCFA agar plates three times, at 24-hour intervals, for purification. Following purification, individual colonies were transferred into 15 ml Falcon tubes containing 14 ml of YCFA broth and incubated at 37° C. for 24 hours. Pure bacterial isolates were archived at −80° C. in cryogenic storage tubes, containing 25% (v/v) sterile glycerol.

### Bacterial Identification

Identification of the picked isolates was performed through 16S rRNA gene amplification via PCR, followed by capillary sequencing. Broad range bacterial 16S gene primers were including used, a forward 7F primer: 5′-AGAGTTTGATYMTGGCTCAG-3′ (SEQ ID NO:87), and a reverse 1510R primer: 5′-ACGGYTACCTTGTTACGACTT-3′ (SEQ ID NO:88). Following sequencing, a BLASTn search was performed, using SILVA_138.1, to define each isolate as either a previously characterised or candidate novel species, with 97% sequence similarity between operational taxonomic units (OTUs) designated as the species-level cut-off.

### Genomic Sequencing

A selection of 463 phylogenetically diverse representative isolates were selected for whole genome sequencing. DNA was extracted using the MP Biomedicals FastDNA SPIN Kit for soil, and sequenced on either the Illumina NextSeq2000 or the Illumina NextSeq550. Sequenced reads were trimmed using Trimmomatic v 0.38 and assembled using SPAdes v 3.13.0. Assembled genomes underwent quality control, looking for completeness (>95%) and contamination (<5%) using CheckM v 1.1.3 (Parks et al., 2014). Genomes which passed quality control were annotated using Prokka v1.13 (Seeman, 2014).

### Metagenomic Sequencing and Genome Comparisons

DNA was also extracted from the whole metagenomic community scraped from a non-confluent agar plate, using the same DNA isolation process.

For candidate genomes of interest, genome annotation files were used as input into Roary v3.13.0 (Page et al., 2015) to identify and align core genes within groups of genomes. Core gene alignments were used to generate maximum likelihood trees using RA×ML v8.2.11 with the GTR GAMMA model (Stamatakis, 2014). Trees were visualized using the interactive tree of life (iTOL). Inter-clade differences in gene presence were determined using the Roary output and examining the data for genes present in every strain of the selected clade and absent from any other strain. The presence of virulence genes within genomes was determined using diamond (Buchfink, et al., 2021) with the VFDB (Liu, et al. 2019) list of virulence proteins, and a percent identity cut-off of >70%.

### RNA Sequencing

RNA was extracted from both patient derived mucosal samples and Caco2 cells post stimulation, using the QIAGEN RNeasy Mini Kit as per the protocol, modified to encompass use of the FastPrep96 high-throughput homogenizer (MP Biomedicals), for the mucosal samples. For sequencing, libraries were generated using an in-house multiplex RNA-seq method (MHTP, Medical Genomics Facility), and paired-end sequencing (R1 19 bp; R2 72 bp) was performed on the NextSeq 550 (Illumina), using the v2.5 High Output Kit. Bcl2fastq (v2.20.0.422, Illumina) was used for base calling.

### Patient RNA Sequencing Analysis

RNA sequencing analysis was performed in R (v3.5.1). The scPipe package (v1.2.1) was employed to process and de-multiplex the data. Read alignment was performed using the RSubread package (v1.30.9). An index was built using the Ensembl Homo sapiens GRCh38 primary assembly genome file and alignment was performed with default settings. Aligned reads were mapped to exons using the sc_exon_mapping function with the Ensembl Homo sapiens GRCh38 v98 GFF3 genome annotation file. The resulting BAM file was de-multiplexed and reads mapping to exons were associated with each individual sample using the sc_demultiplex function, and an overall count for each gene for each was sample was generated using the sc_gene_counting function (with UMI_cor=1). Additional gene annotation was obtained using the biomaRt package (v2.36.1) and a DGEList object was created with the counts and gene annotation using the edgeR package (v3.28.1).

Gene set collections were obtained from the Broad Institute Molecular Signature Database (v5.2) using the EGSEA package (v1.14.0). The HALLMARK_INFLAMMATORY_RESPONSE gene set from the Hallmark gene set collection was used to classify samples as inflamed (IR) or non-inflamed (NR). log₂ CPM expression values for the 166 detected genes inflammatory response genes were used for k-means clustering using the kmeans function from the R stats package with two centres and 100 random starts. Multidimensional scaling plots were generated on log₂ CPM expression values using plotMDS from the limma package (v3.42.0). Pairwise gene selection using the top 500 genes to show the overall distances between samples. Common gene selection using all 166 detected HALLMARK_INFLAMMATORY_RESPONSE genes was used to demonstrate the k-means clustering classification.

Differential expression analysis was performed using limma. Samples were assigned into six groups based on the site (T, C or R) and inflammation status (IR or NR) as determined from the k-means clustering of inflammatory genes. The group and sequencing batch were incorporated into the design matrix. Counts were transformed and associated weights calculated using the voomWithQualityWeights function, to also incorporate sample weights. The voomWithQualityWeights output was used with patient as a blocking factor in the duplicateCorrelation function to calculate the intra-patient consensus correlation. The voomWithQualityWeights values were then recalculated with the blocking factor and consensus correlation included and then the output was used in duplicateCorrelation to update the consensus correlation. A linear model was fit using the ImFit function with the final voomWithQualityWeights output, design matrix, blocking factor and consensus correlation. Inflamed and non-inflamed regions within each intestinal site were compared using the contrasts.fit function.

For differential expression analysis, moderated t-statistics were calculated using the treat function with a 1.2-fold cut-off. Differentially expressed genes were determined using a false discovery rate (FDR) adjusted p-value<0.05. The fry rotational gene set test was used to examine the Caco-2 gene set, incorporating the S149.v.64_8hr log₂ fold change values as gene weights. Either up-and downregulated genes were used or just upregulated genes. Expression values and associated weights were taken from the voomWithQualityWeights output and the block and consensus correlation values were also used, as with ImFit. Gene set enrichment was indicated using the barcode plot function, with the t-statistics recalculated without a fold-change threshold using eBayes. Gene set testing for different contrasts was performed using the cameraPR function using pre-ranked moderated t-statistics (recalculated without a fold change cut-off using the eBayes function, rather than treat). Relative log₂ counts per million (CPM) expression values were used for heat maps. The edgeR cpm function was used to obtain log₂ CPM values. Relative values were obtained for each cell line by subtracting the average log₂ CPM value for the respective untreated samples, obtained using the cpmByGroup function. Heat map scales were truncated as indicated.

Caco2 RNA Sequencing Analysis scPipe, biomaRt and edgeR were used as above. Lowly expressed genes were instead using the filterByExpr function. A linear model was fit on expression values from voom and the design matrix. Differential expression was again performed with treat and a 1.2-fold threshold. The original multiplexed R1 and R2 FASTQ files were de-multiplexed using cutadapt v3.0 (with error rate 1 and action none) and uploaded to . . . with accession number . . . =/*.

### Metagenomic and RNA Sequencing Analysis

Metagenomic reads were analysed using a reference database for taxonomic classification of metagenomes was constructed using 463 high-quality genome sequences isolated from our PIBD cohort, in addition to the complete RefSeq genomes for Archaea, Viruses and Bacteria, a collection of vectors (NCBI's UniVec db) and the Human genome. Taxonomic assignments were performed with the CCMetagen pipeline v.1.2.5 (Marcelino et al., 2020). Reference sequences were indexed with KMA v.1.3.13 (Clausen et al., 2018) using the ‘-NI-Sparse TG’ options. Mapping was performed with KMA using options ‘-1t1-mem_mode-and-apm p-ef’, and taxonomic assignments were obtained with CCMetagen using options ‘-du fr-off y’, which allowed us to obtain abundance estimates in terms of read counts. Species identified by less than 100 sequence reads were excluded from the analyses, and a Center logratio transformation was applied using the MixOmics R package (Rohart et al., 2017). The inventors next sought to identify bacterial species that most correlate with intestinal inflammation (as identified via RNA-Sequencing analysis) using Sparse Partial Least Squares regression (Cao et al., 2009), and the results were visualized with Clustered Image Maps (MixOmics R package).

### Bacterial Preparation for Cell Culture

Bacterial isolates were streaked onto YCFA agar plates and incubated for 24 hours in an anaerobic environment. For each isolate of interest, an individual colony was grown overnight in pre-reduced YCFA broth and then twice pelleted by centrifugation and washed with PBS. Following this, the pellet was resuspended in 1 mL of DMEM, and an optical density reading was obtained.

### Heat Killed Isolates

For experimentation containing heat killed isolates, the bacteria were prepared as above. Once isolates had been washed twice, they were divided into 1 mL aliquots and heated to 99° C. for 60 minutes. Isolates were then plated onto YCFA agar plates and incubated in the anaerobic environment for 24 hours to ensure no viable cells remained.

Correlation of Optical Density with Colony Forming Unit Count

To ensure an accurate multiplicity of infection (MOI) for bacterial stimulations, it was necessary to correlate the optical density (OD) measurements obtained after washing the bacterial pellets, with a colony forming unit count (CFU). Therefore, the bacterial pellets were washed and resuspended as stated above, and then diluted with PBS until an OD₆₀₀ of 1 was obtained. Following this, they were serially diluted down to 10⁻⁶ and plated directly onto YCFA agar. The isolates were allowed to incubate in the anaerobic environment for 24 hours and then enumerated at the dilution factor in which distinct, non-converging colonies were seen. This allowed for a CFU count to be obtained that correlated with an OD₆₀₀ of 1.

### Culture and Maintenance of Human Colonic Adenocarcinoma (Caco2) Cells

The Caco2 epithelial cells (ATCC HTB-37) were maintained in complete Dulbecco's Modified Eagle Media (cDMEM), low glucose, GlutaMAX™ Supplement, pyruvate containing 10% v/v fetal calf serum (FCS). Cells were grown in 175 cm² flasks for 48 hours, in 5% CO₂ at 37° C.

### Seeding Caco2 Cells

For bacterial stimulations, cells were seeded into 6-well plates at 1×10⁶ cells per well, in a final volume of 2 mL per well of cDMEM. Cells were incubated for 48 hours prior to stimulation.

### Serum Starving Caco2 Cells

Caco2 cells were serum starved by replacing cDMEM with unmodified DMEM (low glucose, GlutaMAX™ Supplement, pyruvate), 3 hours prior to stimulation of cells with bacterial isolates.

Stimulation of Caco2 Cells with Bacterial Isolates

Bacterial cultures were prepared as described in “Bacterial preparation”, and stock cultures for each isolate were created to contain 1×10⁷ bacterial cells. The bacterial isolates were then inoculated onto the Caco2 cells at a multiplicity of infection (MOI) of 10:1, by the addition of 200 μl of the bacterial stock solution to the Caco2 cells.

### Collection of Cell Supernatant and Cell Lysates

At 0, 2, 4, 6, 8 and 24 hours post infection, 1 mL of the cellular supernatant was collected from each well. The cellular supernatant was then stored at −80° C. immediately following collection.

At 0, 2, 4, 6, 8 and 24 hours post infection cellular lysates were collected. 350 μl of RLT lysis buffer, including 1% (v/v) 3-mercaptoethanol, was applied to the cells, which were then scraped from the surface of the plate, homogenised via pipette action and immediately stored at −80° C.

### Cell Cytotoxicity: Lactate Dehydrogenase (LDH) Assay

The CytoTox 96® Assay (Promega) was used as per the manufacturer's instructions to determine the LDH release from cells, following stimulation with the bacterial isolates. Additionally, a non-stimulated cell control was used to determine any background cell death, through using cells seeded into a well at the same concentration, without the addition of a bacterial isolate. Finally, a cell culture media control was also used, to account for any impact of the DMEM used.

### Colony Forming Unit Counts

For each sample, 50 μl of the cellular supernatant was collected, diluted by a factor of 10 with sterile pre-reduced 1×PBS, serially diluted down to 10⁻⁶ and plated directly onto YCFA agar. To allow for enumeration of the colony forming unit (CFU) counts between samples, 10 μl aliquots of each dilution factor were plated in triplicate onto YCFA agar. Plates were enumerated following a 24-hour incubation in the anaerobic environment. Calculations were performed to determine the CFU counts per milliliter of cellular supernatant.

### Scanning Electron Microscopy

Images of the bacterial candidates investigated were generated using scanning electron microscopy (SEM). Bacterial isolates were prepared by streaking isolates from previously purified glycerol stocks, stored at −80° C. After 24 hours of incubation in an anaerobic environment, one colony was inoculated into 15 mL of sterile, pre-reduced, YCFA broth and incubated for an additional 24 hours. The culture was then pelleted by centrifugation at 4000×g for 10 minutes. The supernatant was discarded and the pellet was resuspended in 10 ml of sterile 1×PBS. The resuspended pellet was re-pelleted by centrifugation at 4000×g for 10 minutes. This process was repeated twice for each bacterial isolate. Following the second PBS wash, the resuspended pellet was again pelleted by centrifugation at 4000×g for 10 minutes. The pellet was then resuspended in 1 ml of Karnovsky fixative solution and imaged at Monash Micro Imaging.

### Example 2—Identification of Biotherapeutic Strains

A comprehensive bacterial culture collection provides the capacity for cohort specific, reference based metagenomic sequencing and experimental validation (Forster et al., 2019). To build a paediatric inflammatory bowel disease (PIBD) specific culture collection, 286 mucosal biopsies were obtained from 100 paediatric patients (PIBD: 58 patients, control: 42 patients) across three intestinal regions (terminal ileum: 93 samples; caecum: 96 samples; rectum: 97 samples). In total, 6,416 isolates (Bacteroidetes: 3,226 isolates, Firmicutes: 1,480 isolates, Actinobacteria: 370 isolates, Proteobacteria: 1,321 isolates, Fusobacteria: 17 isolates and Verrucomicrobia: 2 isolates), representing 207 distinct species, including 53 novel species were identified through 16S rRNA capillary sequencing. To provide a cohort specific reference genome database for reference based metagenomic analyses, 463 phylogenetically diverse representative isolates were also subjected to whole genome sequencing.

In addition to accurately defining the microbial community, establishing a detailed understanding of the cellular molecular state at specific tissue sites is essential to fully elucidate the host-microbe interactions. While endoscopy, coupled with histological assessment, represents the gold standard in diagnosis of IBD, these methods remain subjective and lack the molecular resolution that may impact microbial composition and interactions at the mucosal layer. To overcome these limitations, the inventors complemented macroscopic and histological assessment with RNA sequencing based transcriptional profiling on 231 matched samples from the terminal ileum, caecum and rectum of 77 patients.

Initially, the top 500 most variable genes across sample pairs were examined, with the intestinal region exhibiting the greatest influence on gene expression, followed by the samples' histologically defined inflammatory status. However, while the inflammatory state of the sample, defined histologically, is independently related to the variation in gene expression seen across samples, there were a number of histologically inflamed samples clustering with the non-inflamed. Therefore, the inventors sought to define a molecular inflammatory state using k-means consensus clustering, employing genes from the Hallmark inflammatory gene set (Liberzon et al., 2015), to define inflammation. This method identified 69 molecularly inflamed (IBD: 58, non-IBD: 11) and 162 molecularly non-inflamed (IBD: 74, non-IBD: 88) samples. Overall, 51 IBD samples (39%) and 10 non-IBD samples (10%) were re-categorised from their histological classification according to their molecular profiles, with classification henceforth based on this molecular state.

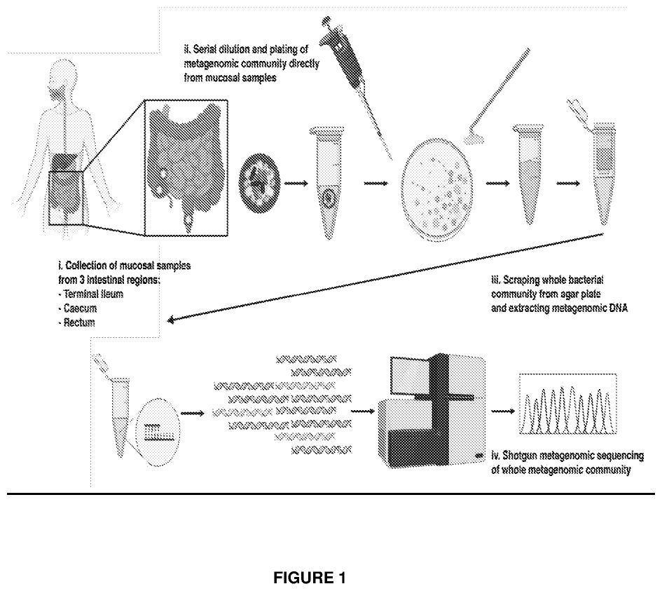

Having established a comprehensive, genome sequenced culture collection and accurate classification of molecular inflammatory state within biopsy samples, the inventors next sought to understand the microbial composition within these 231 intestinal samples. While shotgun metagenomic sequencing can provide high resolution measurement at the site of disease, the dominance of human DNA within biopsy samples has limited the application of this best practice approach. To overcome this challenge, the inventors applied a culture based, eukaryotic DNA depletion process, to remove human DNA and allow shotgun metagenomic sequencing on these samples (FIG. 1). This approach resulted in a median eukaryotic DNA representation of 0.01% of the total sample DNA composition (maximum: 0.03%, minimum: 0%) and provided metagenomic sequencing data suitable for detailed reference based metagenomic analysis.

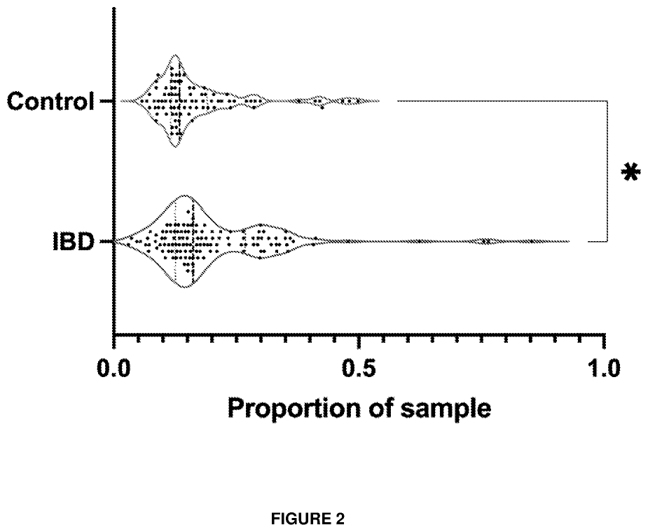

To understand the host-microbe interactions that underpin phenotypic variations in disease manifestation, the inventors undertook integrated analysis of the metagenomic and transcriptional sequencing in the context of our 463 cohort specific, high quality reference genomes and the RefSeq reference dataset (O'Leary et al., 2016). This allowed for identification of bacterial clades associated with upregulation of inflammation associated transcriptional pathways, including TNFα signalling via NF-κB (P=1.6×10⁻²²), IFNα (P=2.8×10⁻³⁴) and IFNγ (P=2.3×10⁻⁵⁰) response, IL6 JAK STAT3 signalling (P=1×10⁻³⁹), and other Hallmark inflammatory response genes (P=4.9 ×10⁻³³) (Liberzon et al., 2015), while accounting for the compositional nature of the microbiome species abundance data. Notably, known health associated bacteria, including members of the Bacteroides, Parabacteroides and Phocaeicola genera were negatively correlated with genes associated with inflammation, as defined by Hallmark inflammatory signatures, while pathogens and opportunistic pathogens, including members of the Klebsiella and Salmonella genera, and Clostridium perfringens exhibited a positive correlation with inflammation associated Hallmark signatures (Liberzon et al., 2015). In addition to these common pathogens, this analysis also identified an Enterococcus clade positively correlated with inflammatory signalling. Detailed analysis of the Enterococcus isolates cultured from this cohort identified a subclade enriched in IBD patients (P=0.0077) and a clade with equal representation in both control and IBD patients (FIG. 2).

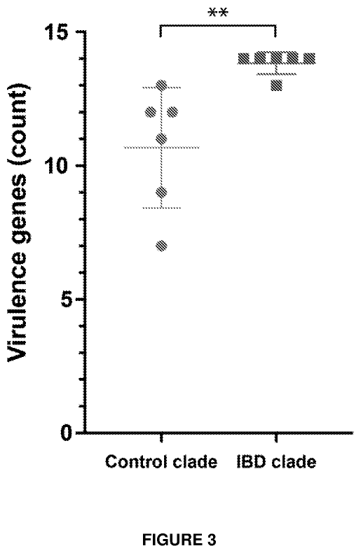

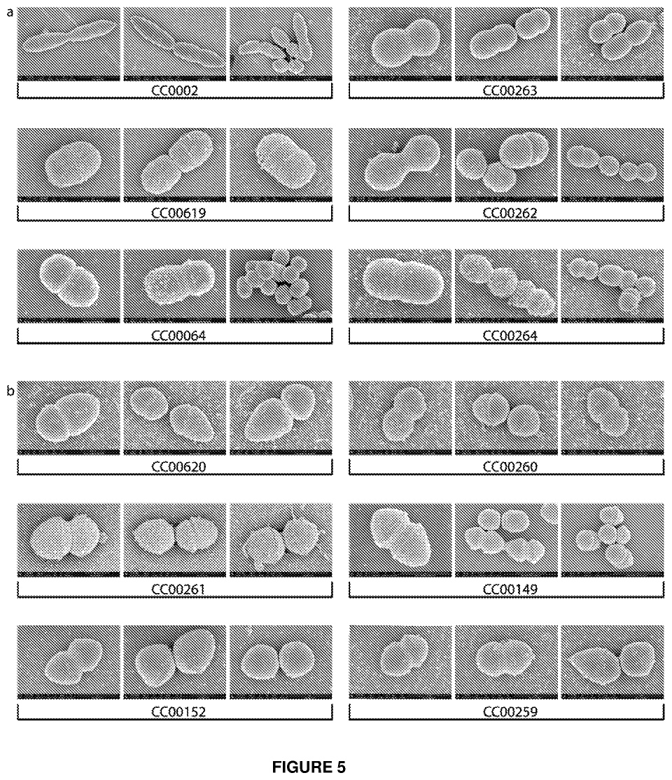

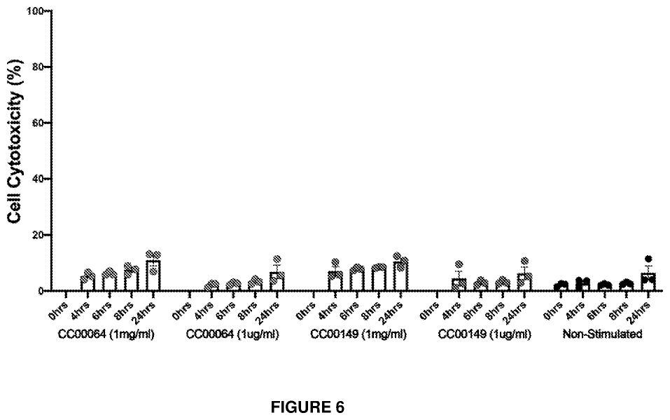

Six representative bacterial isolates with matched genome sequences were selected from the cohort specific, bacterial culture collection (n=12, 6 IBD-associated clade, 6 control clade) to undertake isolate level genomic analysis and phenotypic characterisation. Consistent with a more severe disease phenotype, genomic assessment of virulence factors in the IBD-associated and control clades identified a higher number of virulence genes within the IBD clade (median: 14 genes) compared to the control clade (median: 11.5 genes, P<0.01; Mann-Whitney U test) (FIG. 3). These observations are consistent with previous data in smaller cohorts that predicted Enterococci from IBD patients possess more virulence factors than those in control patients (Golińska et al., 2013). To validate these genomic predictions, cell cytotoxicity, as determined via lactate dehydrogenase (LDH) release into the extracellular medium, was assessed following a 24 hour stimulation of Caco2 intestinal epithelial cells with the bacterial candidates (multiplicity of infection of 10). Contrary to the genomic predictions, isolates from the control clade induced a significantly greater cell cytotoxicity (mean=42.59%) than isolates within the IBD-associated clade (mean=11.14%, P=0.0006; Ratio paired t-test) (FIG. 4). Notably, no clade-specific morphological differences were detected by scanning electron microscopy (SEM) (FIGS. 4 and 5) and cytotoxicity was lost following heat treatment, suggesting the observed phenotype is dependent on bacterial viability (FIG. 6).

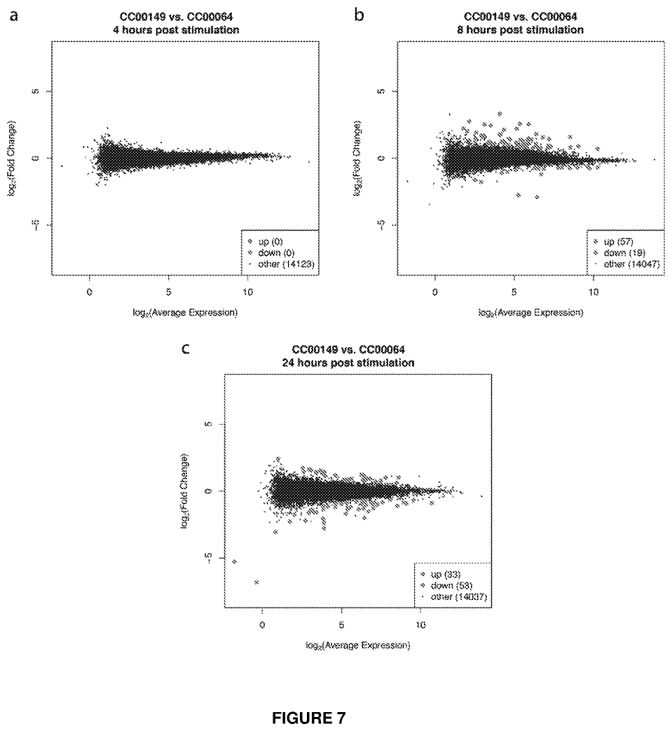

To further investigate the host-microbe interactions, isolates CC00149 (IBD-associated clade) and CC00064 (control clade) were selected as representative isolates from the subclades identified. RNA sequencing of the transcriptional response to each of these isolates in Caco2 cells was performed at 4, 8 and 24 hours post-stimulation. While the majority of differentially expressed genes were induced in response to both isolates and no differences were observed at 4 hours, 76 isolate specific genes were differentially expressed at 8 hours (57 increased in CC00149, 19 increased in CC00064) (Table 1) and 86 genes were differentially expressed at 24 hours (33 increased in CC00149, 53 increased in CC00064) (Table 2; FIG. 7). Additionally, the Hallmark hypoxic response was most significantly upregulated in response to stimulation with CC00064 (P=1.41×10⁻¹⁰) and the Hallmark TNFα signalling via NF-κB response was most significantly upregulated in response to CC00149 (P=7.9×10⁻¹⁵⁾at 8 hours post-stimulation (Liberzon et al., 2015) (Table 3).

To understand the relationship between clade-specific transcriptional changes and cell cytotoxicity observed at 24 hours, the inventors focused on the 76 genes that were either significantly upregulated or downregulated in CC00149 relative to CC00064 at 8 hours post-stimulation. Differences at this time point could represent key responses without confounding factors associated with extensive cell cytotoxicity at 24 hours. Consistent with this hypothesis, gene set testing identified TNFα signalling via NF-κB (P=7.3×10⁻¹⁶), activation of pro-apoptotic pathways (P=2.91×10⁻²), and initiation of the unfolded protein response (P=2.42×10⁻⁷) to be significantly upregulated in response to CC00149, compared to CC00064 (Table 4).

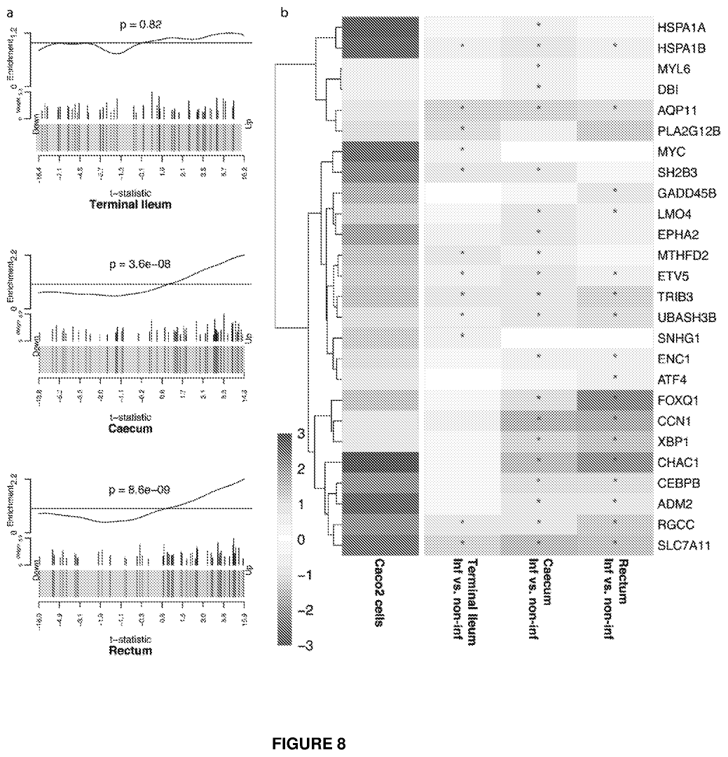

To assess the clinical relevance of these key clade-specific responses, the inventors next compared the transcriptional profile measured in our Caco2 in vitro model to those observed in the mucosal samples (n=231) from our patient cohort (n=77). Focusing on the 76 genes that were significantly differentially expressed in the in vitro Caco2 cells at 8 hours, within the patient cohort dataset, a significant enrichment was observed in patient biopsy samples derived from both the caecum (P=3.6×10⁻⁸) and rectum (P=8.6×10⁻⁹). However, no such relationship was detected in the terminal ileum (P=0.82) (FIG. 8), which may be explained by the colonic origin of the Caco2 cells in our co-culture model. In total, 34.2% (n=26) of the 76 genes were statistically differentially expressed and exhibited the same direction of expression change (20 upregulated, 6 downregulated) in both the Caco2 model system and the primary biopsy samples (FIG. 8). Importantly, these included genes known to be associated with cell death and the unfolded protein response including CHAC1 (P=1.53×10⁻⁸), CEBPB (P=3.37×10⁻⁷), TRIB3 (P=5.25×10⁻⁶), PPP1R15A (P=0.011), DDIT3 (P=4.5×10⁻⁵), ATF4 (P=0.020) and XBP1 (P=0.043). Taken together, these results suggest a clade-specific differential regulation of cell death pathways associated with IBD mucosal immune state that may be suitable for clinical intervention in selected patients.

In total, 44 new strains of a new clade, referred herein to clade 149, of non-inflammatory Enterococcus were identified represented by the 16s rRNA gene sequences provided as SEQ ID NO's 1 to 44, and including Enterococcus sp. CC00149 deposited under V19/018754, and Enterococcus sp. CC00259 deposited under V19/018755, on 9 Sep. 2019 at the National Measurement Institute, Australia, as well as Enterococcus sp. CC00620 deposited under V21/013048 on 29 Jun. 2021 at the National Measurement Institute, Australia. Furthermore, 30 new strains of a new clade, referred herein to clade 64, of inflammatory Enterococcus, closely related to the above-mentioned non-inflammatory Enterococcus, were identified represented by the 16s rRNA gene sequences provided as SEQ ID NO's 45 to 74, and including Enterococcus sp. CC00064 deposited under V21/013046, and Enterococcus sp. CC00619 deposited under V21/013047, on 29 Jun. 2021 at the National Measurement Institute, Australia, as well as Enterococcus sp. CC00262 deposited under V20/006238 on 18 Mar. 2020 at the National Measurement Institute, Australia and Enterococcus sp. CC0002 deposited under V21/014119 on 20 Jul. 2021 at the National Measurement Institute, Australia.

TABLE 1
Differentially expressed genes at 8 hours.

| | | | CC00149 | | |
|---|---|---|---|---|---|
| | | | v. | CC00149 v. | CC00149 |
| | | Chro- | CC00064 | CC00064 | v. |
| | | mo- | 8 hr Fold | 8 hr | CC00064 |
| | External | some | change | Adjusted | 8 hr |
| Ensembl gene ID | gene name | name | (log) | p-value | Results |
| ENSG00000128965 | CHAC1 | 15 | 3.320 | 1.53E−08 | 1 |
| ENSG00000102760 | RGCC | 13 | 2.123 | 7.09402E−08 | 1 |
| ENSG00000172216 | CEBPB | 20 | 2.208 | 3.37E−07 | 1 |
| ENSG00000065911 | MTHFD2 | 2 | 1.438 | 7.73115E−07 | 1 |
| ENSG00000136997 | MYC | 8 | 2.574 | 9.22773E−07 | 1 |
| ENSG00000204389 | HSPA1A | 6 | −2.880 | 1.20134E−06 | −1 |
| ENSG00000109321 | AREG | 4 | 1.596 | 1.20314E−06 | 1 |
| ENSG00000204388 | HSPA1B | 6 | −2.734 | 1.20314E−06 | −1 |
| ENSG00000101255 | TRIB3 | 20 | 1.463 | 5.25E−06 | 1 |
| ENSG00000164379 | FOXQ1 | 6 | 1.396 | 5.32347E−06 | 1 |
| ENSG00000139514 | SLC7A1 | 13 | 1.426 | 7.05406E−06 | 1 |
| ENSG00000143013 | LMO4 | 1 | 1.368 | 8.19597E−06 | 1 |
| ENSG00000162772 | ATF3 | 1 | 2.523 | 1.60648E−05 | 1 |
| ENSG00000113739 | STC2 | 5 | 1.554 | 1.60648E−05 | 1 |
| ENSG00000116044 | NFE2L2 | 2 | 1.415 | 1.60648E−05 | 1 |
| ENSG00000175197 | DDIT3 | 12 | 1.806 | 4.50068E−05 | 1 |
| ENSG00000006652 | IFRD1 | 7 | 1.428 | 8.93292E−05 | 1 |
| ENSG00000168003 | SLC3A2 | 11 | 0.889 | 0.00011553 | 1 |
| ENSG00000232956 | SNHG15 | 7 | 1.102 | 0.00020093 | 1 |
| ENSG00000148677 | ANKRD1 | 10 | 2.533 | 0.000659475 | 1 |
| ENSG00000165732 | DDX21 | 10 | 0.794 | 0.00068938 | 1 |
| ENSG00000254166 | CASC19 | 8 | 1.672 | 0.000825148 | 1 |
| ENSG00000163811 | WDR43 | 2 | 0.936 | 0.001142555 | 1 |
| ENSG00000134294 | SLC38A2 | 12 | 0.851 | 0.001241549 | 1 |
| ENSG00000130766 | SESN2 | 1 | 1.810 | 0.001433601 | 1 |
| ENSG00000255717 | SNHG1 | 11 | 1.294 | 0.00145339 | 1 |
| ENSG00000006327 | TNFRSF12A | 16 | 1.410 | 0.002130909 | 1 |
| ENSG00000048162 | NOP16 | 5 | 0.836 | 0.003641507 | 1 |
| ENSG00000124882 | EREG | 4 | 1.724 | 0.003641507 | 1 |
| ENSG00000132002 | DNAJB1 | 19 | −1.195 | 0.003641507 | −1 |
| ENSG00000173812 | EIF1 | 17 | 0.686 | 0.004526655 | 1 |
| ENSG00000178301 | AQP11 | 11 | −0.852 | 0.004575365 | −1 |
| ENSG00000151012 | SLC7A11 | 4 | 2.458 | 0.005143655 | 1 |
| ENSG00000107815 | TWNK | 10 | 1.156 | 0.005972688 | 1 |
| ENSG00000129474 | AJUBA | 14 | 1.161 | 0.006114822 | 1 |
| ENSG00000128283 | CDC42EP1 | 22 | 1.274 | 0.006542725 | 1 |
| ENSG00000187653 | TMSB4XP8 | 4 | −1.004 | 0.006542725 | −1 |
| ENSG00000100387 | RBX1 | 22 | −0.710 | 0.007833835 | −1 |
| ENSG00000128165 | ADM2 | 22 | 2.737 | 0.008064961 | 1 |
| ENSG00000099860 | GADD45B | 19 | 1.707 | 0.009605597 | 1 |
| ENSG00000092841 | MYL6 | 12 | −0.658 | 0.009605597 | −1 |
| ENSG00000171552 | BCL2L1 | 20 | 0.745 | 0.011048874 | 1 |
| ENSG00000087074 | PPP1R15A | 19 | 1.097 | 0.011048874 | 1 |
| ENSG00000168672 | LRATD2 | 8 | 1.923 | 0.011048874 | 1 |
| ENSG00000232917 | HSPE1P6 | 1 | −1.610 | 0.011048874 | −1 |
| ENSG00000177700 | POLR2L | 11 | −0.720 | 0.011048874 | −1 |
| ENSG00000130332 | LSM7 | 19 | −0.695 | 0.012073981 | −1 |
| ENSG00000170871 | KIAA0232 | 4 | 1.228 | 0.013552942 | 1 |
| ENSG00000142409 | ZNF787 | 19 | 1.217 | 0.013552942 | 1 |
| ENSG00000111252 | SH2B3 | 12 | 2.231 | 0.014378908 | 1 |
| ENSG00000125968 | ID1 | 20 | 1.027 | 0.015373449 | 1 |
| ENSG00000198937 | CCDC167 | 6 | −0.727 | 0.015373449 | −1 |
| ENSG00000196154 | S100A4 | 1 | −0.794 | 0.016580549 | −1 |
| ENSG00000171617 | ENC1 | 5 | 1.067 | 0.018478419 | 1 |
| ENSG00000142871 | CCN1 | 1 | 0.956 | 0.01880577 | 1 |
| ENSG00000229833 | PET100 | 19 | −0.701 | 0.01880577 | −1 |
| ENSG00000117450 | PRDX1 | 1 | −0.689 | 0.01880577 | −1 |
| ENSG00000128272 | ATF4 | 22 | 0.751 | 0.019749229 | 1 |
| ENSG00000102554 | KLF5 | 13 | 1.026 | 0.021011553 | 1 |
| ENSG00000142627 | EPHA2 | 1 | 2.017 | 0.021131987 | 1 |
| ENSG00000155368 | DBI | 2 | −0.576 | 0.021131987 | −1 |
| ENSG00000109971 | HSPA8 | 11 | −0.733 | 0.02425944 | −1 |
| ENSG00000138308 | PLA2G12B | 10 | −1.065 | 0.028930798 | −1 |
| ENSG00000154127 | UBASH3B | 11 | 1.080 | 0.029487821 | 1 |
| ENSG00000185551 | NR2F2 | 15 | −0.780 | 0.029487821 | −1 |
| ENSG00000117597 | UTP25 | 1 | 1.135 | 0.029736817 | 1 |
| ENSG00000111845 | PAK1IP1 | 6 | 0.792 | 0.029736817 | 1 |
| ENSG00000116698 | SMG7 | 1 | 0.721 | 0.037774652 | 1 |
| ENSG00000163072 | NOSTRIN | 2 | 1.482 | 0.037774652 | 1 |
| ENSG00000186352 | ANKRD37 | 4 | 1.762 | 0.037774652 | −1 |
| ENSG00000108509 | CAMTA2 | 17 | 1.592 | 0.038350168 | 1 |
| ENSG00000179041 | RRS1 | 8 | 1.009 | 0.039145256 | 1 |
| ENSG00000244405 | ETV5 | 3 | 1.326 | 0.039518032 | 1 |
| ENSG00000125846 | ZNF133 | 20 | 1.183 | 0.041706425 | 1 |
| ENSG00000100219 | XBP1 | 22 | 0.756 | 0.043039029 | 1 |
| ENSG00000241258 | CRCP | 7 | 0.890 | 0.043246925 | 1 |

TABLE 2
Differentially expressed genes at 24 hours.

| | | | CC00149 | | |
|---|---|---|---|---|---|
| | | | v. | CC00149 | |
| | | | CC00064 | v. | CC00149 |
| | | Chro- | 24 hr | CC00064 | v. |
| | External | mo | Fold | 24 hr | CC00064 |
| | gene | some | change | Adjusted | 24 hr |
| Ensembl gene ID | name | name | (log) | p-value | Results |
| ENSG00000130066 | SAT1 | X | 1.296 | 2.38427E−05 | −1 |
| ENSG00000173110 | HSPA6 | 1 | −6.819 | 2.5767E−05 | −1 |
| ENSG00000256162 | SMLR1 | 6 | 1.238 | 2.5767E−05 | 1 |
| ENSG00000136527 | TRA2B | 3 | 1.009 | 2.5767E−05 | −1 |
| ENSG00000132002 | DNAJB1 | 19 | −1.522 | 0.000168142 | −1 |
| ENSG00000225217 | HSPA7 | 1 | −5.262 | 0.000296923 | −1 |
| ENSG00000287001 | AC010624.5 | 19 | −2.791 | 0.000313668 | −1 |
| ENSG00000105398 | SULT2A1 | 19 | 1.539 | 0.000406171 | 1 |
| ENSG00000148803 | FUOM | 10 | −0.841 | 0.000406171 | −1 |
| ENSG00000116741 | RGS2 | 1 | −2.068 | 0.000546931 | −1 |
| ENSG00000204389 | HSPA1A | 6 | −2.000 | 0.000644689 | −1 |
| ENSG00000196139 | AKR1C3 | 10 | 0.950 | 0.000644689 | 1 |
| ENSG00000109846 | CRYAB | 11 | −2.184 | 0.000735448 | −1 |
| ENSG00000215859 | PDZK1P1 | 1 | 0.863 | 0.001495707 | 1 |
| ENSG00000210077 | MT-TV | MT | −1.333 | 0.001770699 | −1 |
| ENSG00000204388 | HSPA1B | 6 | −1.702 | 0.001996839 | −1 |
| ENSG00000099860 | GADD45B | 19 | −1.636 | 0.001996839 | −1 |
| ENSG00000023608 | SNAPC1 | 14 | −1.579 | 0.001996839 | −1 |
| ENSG00000272055 | RNU6-6P | 10 | −2.388 | 0.001996839 | −1 |
| ENSG00000044574 | HSPA5 | 9 | −0.852 | 0.001996839 | −1 |
| ENSG00000142871 | CCN1 | 1 | −1.180 | 0.002557608 | −1 |
| ENSG00000109321 | AREG | 4 | −1.091 | 0.002711501 | −1 |
| ENSG00000111011 | RSRC2 | 12 | −0.994 | 0.002949491 | −1 |
| ENSG00000113657 | DPYSL3 | 5 | 0.932 | 0.003256039 | 1 |
| ENSG00000162817 | C1orf115 | 1 | 0.934 | 0.00369742 | 1 |
| ENSG00000175197 | DDIT3 | 12 | −1.315 | 0.003739606 | −1 |
| ENSG00000243955 | GSTA1 | 6 | 0.998 | 0.003739606 | 1 |
| ENSG00000165272 | AQP3 | 9 | 1.145 | 0.003739606 | 1 |
| ENSG00000015413 | DPEP1 | 16 | −0.971 | 0.003739606 | −1 |
| ENSG00000166165 | CKB | 14 | −0.915 | 0.003739606 | −1 |
| ENSG00000261701 | HPR | 16 | 1.435 | 0.003739606 | 1 |
| ENSG00000178301 | AQP11 | 11 | 0.862 | 0.003772117 | 1 |
| ENSG00000244067 | GSTA2 | 6 | 0.794 | 0.00379412 | 1 |
| ENSG00000196227 | FAM217B | 20 | 0.823 | 0.003862984 | 1 |
| ENSG00000206633 | SNORA80B | 2 | 1.438 | 0.004146466 | 1 |
| ENSG00000079689 | SCGN | 6 | 1.165 | 0.005250167 | 1 |
| ENSG00000185551 | NR2F2 | 15 | 0.874 | 0.005349455 | 1 |
| ENSG00000169562 | GJB1 | X | 0.714 | 0.006643453 | 1 |
| ENSG00000173262 | SLC2A14 | 12 | −0.987 | 0.006771406 | −1 |
| ENSG00000171557 | FGG | 4 | 1.012 | 0.009030038 | 1 |
| ENSG00000005448 | WDR54 | 2 | −1.194 | 0.009271831 | −1 |
| ENSG00000172296 | SPTLC3 | 20 | 1.346 | 0.009706215 | 1 |
| ENSG00000113361 | CDH6 | 5 | 1.628 | 0.010048968 | 1 |
| ENSG00000186318 | BACE1 | 11 | 1.331 | 0.010048968 | 1 |
| ENSG00000106211 | HSPB1 | 7 | −0.821 | 0.0104085 | −1 |
| ENSG00000068615 | REEP1 | 2 | 1.143 | 0.0104085 | 1 |
| ENSG00000202538 | RNU4-2 | 12 | −3.052 | 0.010720738 | −1 |
| ENSG00000182919 | C11orf54 | 11 | 1.528 | 0.011715246 | 1 |
| ENSG00000206972 | RNU6-17P | 15 | −2.255 | 0.012802014 | −1 |
| ENSG00000225648 | SBDSP1 | 7 | −1.132 | 0.012837285 | −1 |
| ENSG00000171560 | FGA | 4 | 1.121 | 0.012837285 | 1 |
| ENSG00000173145 | NOC3L | 10 | −0.760 | 0.012933969 | −1 |
| ENSG00000077044 | DGKD | 2 | −0.870 | 0.012933969 | −1 |
| ENSG00000131981 | LGALS3 | 14 | −0.700 | 0.012933969 | −1 |
| ENSG00000064787 | BCAS1 | 20 | 1.714 | 0.01367928 | 1 |
| ENSG00000178988 | MRFAP1L1 | 4 | 0.822 | 0.01367928 | 1 |
| ENSG00000135451 | TROAP | 12 | −0.768 | 0.01367928 | −1 |
| ENSG00000120694 | HSPH1 | 13 | −1.027 | 0.015185847 | −1 |
| ENSG00000133226 | SRRM1 | 1 | −0.647 | 0.01603112 | −1 |
| ENSG00000156510 | HKDC1 | 10 | −0.854 | 0.016911303 | −1 |
| ENSG00000224722 | AC020688.1 | 8 | −1.372 | 0.017014725 | −1 |
| ENSG00000244187 | TMEM141 | 9 | −0.823 | 0.018354977 | −1 |
| ENSG00000275713 | H2BC9 | 6 | −1.109 | 0.01862783 | −1 |
| ENSG00000105617 | LENG1 | 19 | −0.922 | 0.019100994 | −1 |
| ENSG00000059804 | SLC2A3 | 12 | −0.878 | 0.020154621 | −1 |
| ENSG00000131876 | SNRPA1 | 15 | −0.717 | 0.020641361 | −1 |
| ENSG00000212901 | KRTAP3-1 | 17 | −1.570 | 0.020671304 | −1 |
| ENSG00000029993 | HMGB3 | X | 0.667 | 0.023294979 | 1 |
| ENSG00000162772 | ATF3 | 1 | −1.359 | 0.024183362 | −1 |
| ENSG00000214049 | UCA1 | 19 | −1.418 | 0.025203845 | −1 |
| ENSG00000232956 | SNHG15 | 7 | −0.805 | 0.028547 | −1 |
| ENSG00000128228 | SDF2L1 | 22 | −0.736 | 0.028963982 | −1 |
| ENSG00000016391 | CHDH | 3 | 0.846 | 0.02950772 | 1 |
| ENSG00000236493 | EIF2S2P3 | 10 | −0.710 | 0.031399145 | −1 |
| ENSG00000117394 | SLC2A1 | 1 | −1.000 | 0.032077389 | −1 |
| ENSG00000173540 | GMPPB | 3 | −1.447 | 0.032077389 | −1 |
| ENSG00000197142 | ACSL5 | 10 | 0.750 | 0.032535675 | 1 |
| ENSG00000148926 | ADM | 11 | −1.350 | 0.035815324 | −1 |
| ENSG00000164938 | TP53INP1 | 8 | 1.415 | 0.035815324 | 1 |
| ENSG00000255717 | SNHG1 | 11 | 0.908 | 0.036902414 | 1 |
| ENSG00000170477 | KRT4 | 12 | 2.422 | 0.036902414 | 1 |
| ENSG00000122884 | P4HA1 | 10 | −0.910 | 0.043337721 | −1 |
| ENSG00000138207 | RBP4 | 10 | 0.617 | 0.045214266 | 1 |
| ENSG00000188037 | CLCN1 | 7 | −1.767 | 0.046413879 | −1 |
| ENSG00000178381 | ZFAND2A | 7 | −0.788 | 0.046521684 | −1 |
| ENSG00000175352 | NRIP3 | 11 | 0.830 | 0.049520659 | 1 |

TABLE 3
Hallmark responses.

| | | Genes. | S64_ | S149_ | S64_ | S149_ | S64_ | S149_ |
|---|---|---|---|---|---|---|---|---|
| | Set. | Ex- | 8 hr.avg. | 8 hr.avg. | 8 hr. | 8 hr. | 8 hr. | 8 hr. |
| MSigDB.Gene.Set | Size | pressed | logFC | logFC | PValue | PValue | FDR | FDR |
| HALLMARK_HYPOXIA | 200 | 150 | 0.399 | 0.413 | 2.82E−12 | 3.44E−08 | 1.41E−10 | 5.73E−07 |
| HALLMARK_MYC_TARGETS_V1 | 200 | 199 | −0.158 | −0.153 | 1.45E−10 | 2.93E−08 | 3.62E−09 | 5.73E−07 |
| HALLMARK_MYC_TARGETS_V2 | 58 | 57 | −0.227 | 0.040 | 1.73E−07 | 9.32E−01 | 2.88E−06 | 9.44E−01 |
| HALLMARK_CHOLESTEROL_HOMEOSTASIS | 74 | 64 | 0.181 | 0.189 | 8.14E−07 | 5.83E−04 | 1.02E−05 | 3.86E−03 |
| HALLMARK_GLYCOLYSIS | 200 | 163 | 0.182 | 0.158 | 2.26E−03 | 1.45E−01 | 2.26E−02 | 3.17E−01 |
| HALLMARK_BILE_ACID_METABOLISM | 112 | 73 | 0.129 | 0.069 | 5.05E−03 | 4.74E−01 | 4.21E−02 | 6.44E−01 |
| HALLMARK_E2F_TARGETS | 200 | 200 | −0.067 | −0.059 | 2.12E−02 | 3.84E−02 | 1.30E−01 | 1.08E−01 |
| HALLMARK_G2M_CHECKPOINT | 200 | 196 | −0.065 | −0.026 | 2.46E−02 | 2.46E−01 | 1.30E−01 | 4.55E−01 |
| HALLMARK_FATTY_ACID_METABOLISM | 158 | 130 | 0.105 | −0.002 | 2.61E−02 | 3.53E−01 | 1.30E−01 | 5.88E−01 |
| HALLMARK_PEROXISOME | 104 | 88 | 0.101 | 0.020 | 2.31E−02 | 9.44E−01 | 1.30E−01 | 9.44E−01 |
| HALLMARK_INTERFERON_GAMMA_RESPONSE | 200 | 116 | 0.116 | 0.110 | 3.21E−02 | 2.30E−02 | 1.46E−01 | 8.22E−02 |
| HALLMARK_WNT_BETA_CATENIN_SIGNALING | 42 | 31 | −0.166 | −0.026 | 3.66E−02 | 7.30E−01 | 1.53E−01 | 8.29E−01 |
| HALLMARK_KRAS_SIGNALING_UP | 200 | 96 | 0.117 | 0.290 | 5.23E−02 | 6.17E−04 | 1.89E−01 | 3.86E−03 |
| HALLMARK_INTERFERON_ALPHA_RESPONSE | 97 | 64 | 0.144 | 0.080 | 5.30E−02 | 2.33E−01 | 1.89E−01 | 4.48E−01 |
| HALLMARK_TNFA_SIGNALING_VIA_NFKB | 200 | 148 | 0.119 | 0.573 | 1.07E−01 | 1.57E−16 | 3.40E−01 | 7.87E−15 |
| HALLMARK_APICAL_SURFACE | 44 | 22 | 0.115 | 0.003 | 1.09E−01 | 6.12E−01 | 3.40E−01 | 7.46E−01 |
| HALLMARK_SPERMATOGENESIS | 135 | 63 | 0.074 | 0.069 | 1.41E−01 | 1.74E−01 | 4.16E−01 | 3.62E−01 |
| HALLMARK_DNA_REPAIR | 150 | 141 | −0.038 | −0.076 | 1.80E−01 | 4.90E−02 | 4.99E−01 | 1.29E−01 |
| HALLMARK_UNFOLDED_PROTEIN_RESPONSE | 113 | 107 | −0.025 | 0.197 | 1.91E−01 | 3.99E−04 | 5.03E−01 | 3.33E−03 |
| HALLMARK_OXIDATIVE_PHOSPHORYLATION | 200 | 195 | −0.020 | −0.128 | 2.02E−01 | 2.21E−07 | 5.04E−01 | 2.76E−06 |
| HALLMARK_INFLAMMATORY_RESPONSE | 200 | 91 | 0.052 | 0.268 | 2.49E−01 | 1.04E−04 | 5.49E−01 | 1.04E−03 |
| HALLMARK_TGF_BETA_SIGNALING | 54 | 48 | −0.099 | 0.199 | 2.46E−01 | 3.38E−02 | 5.49E−01 | 1.08E−01 |
| HALLMARK_COMPLEMENT | 200 | 129 | 0.085 | 0.129 | 2.53E−01 | 2.01E−01 | 5.49E−01 | 4.02E−01 |
| HALLMARK_XENOBIOTIC_METABOLISM | 200 | 147 | 0.057 | −0.005 | 2.79E−01 | 4.57E−01 | 5.58E−01 | 6.44E−01 |
| HALLMARK_PROTEIN_SECRETION | 96 | 92 | 0.055 | 0.017 | 2.77E−01 | 9.29E−01 | 5.58E−01 | 9.44E−01 |
| HALLMARK_UV_RESPONSE_DN | 144 | 114 | 0.065 | 0.215 | 3.05E−01 | 8.77E−04 | 5.87E−01 | 4.38E−03 |
| HALLMARK_P53_PATHWAY | 200 | 170 | 0.047 | 0.186 | 3.39E−01 | 1.05E−02 | 5.98E−01 | 4.38E−02 |
| HALLMARK_MTORC1_SIGNALING | 200 | 189 | 0.104 | 0.123 | 3.47E−01 | 3.35E−01 | 5.98E−01 | 5.88E−01 |
| HALLMARK_UV_RESPONSE_UP | 158 | 129 | 0.004 | 0.066 | 3.46E−01 | 6.33E−01 | 5.98E−01 | 7.54E−01 |
| HALLMARK_ANGIOGENESIS | 36 | 24 | −0.047 | 0.041 | 3.92E−01 | 8.79E−01 | 6.53E−01 | 9.44E−01 |
| HALLMARK_IL2_STAT5_SIGNALING | 199 | 133 | 0.092 | 0.243 | 4.66E−01 | 8.33E−04 | 7.28E−01 | 4.38E−03 |
| HALLMARK_ESTROGEN_RESPONSE_EARLY | 200 | 156 | 0.055 | 0.114 | 4.63E−01 | 9.31E−02 | 7.28E−01 | 2.22E−01 |
| HALLMARK_IL6_JAK_STAT3_SIGNALING | 87 | 47 | −0.007 | 0.239 | 5.18E−01 | 3.89E−02 | 7.62E−01 | 1.08E−01 |
| HALLMARK_MYOGENESIS | 200 | 100 | 0.048 | 0.042 | 5.05E−01 | 5.08E−01 | 7.62E−01 | 6.69E−01 |
| HALLMARK_APOPTOSIS | 161 | 125 | 0.018 | 0.174 | 5.89E−01 | 8.10E−03 | 7.77E−01 | 3.68E−02 |
| HALLMARK_MITOTIC_SPINDLE | 199 | 195 | 0.006 | 0.104 | 5.88E−01 | 2.11E−02 | 7.77E−01 | 8.11E−02 |
| HALLMARK_ADIPOGENESIS | 200 | 171 | 0.046 | −0.016 | 5.72E−01 | 1.46E−01 | 7.77E−01 | 3.17E−01 |
| HALLMARK_PI3K_AKT_MTOR_SIGNALING | 105 | 88 | 0.010 | 0.071 | 6.06E−01 | 5.74E−01 | 7.77E−01 | 7.18E−01 |
| HALLMARK_ESTROGEN_RESPONSE_LATE | 200 | 152 | 0.034 | 0.018 | 6.01E−01 | 7.14E−01 | 7.77E−01 | 8.29E−01 |
| HALLMARK_KRAS_SIGNALING_DN | 200 | 68 | 0.035 | −0.082 | 6.82E−01 | 4.16E−01 | 8.53E−01 | 6.30E−01 |
| HALLMARK_REACTIVE_OXYGEN_SPECIES_PATHWAY | 49 | 42 | −0.016 | −0.028 | 7.80E−01 | 3.59E−02 | 9.27E−01 | 1.08E−01 |
| HALLMARK_ALLOGRAFT_REJECTION | 200 | 86 | 0.017 | 0.049 | 7.97E−01 | 3.71E−01 | 9.27E−01 | 5.99E−01 |
| HALLMARK_PANCREAS_BETA_CELLS | 40 | 15 | 0.106 | 0.008 | 7.90E−01 | 4.30E−01 | 9.27E−01 | 6.33E−01 |
| HALLMARK_HEDGEHOG_SIGNALING | 36 | 22 | 0.018 | 0.114 | 8.22E−01 | 3.44E−01 | 9.34E−01 | 5.88E−01 |
| HALLMARK_EPITHELIAL_MESENCHYMAL_TRANSITION | 200 | 114 | 0.014 | 0.129 | 8.85E−01 | 8.19E−02 | 9.42E−01 | 2.05E−01 |
| HALLMARK_HEME_METABOLISM | 200 | 153 | 0.013 | 0.054 | 8.84E−01 | 3.96E−01 | 9.42E−01 | 6.19E−01 |
| HALLMARK_APICAL_JUNCTION | 200 | 125 | −0.005 | 0.047 | 8.98E−01 | 4.77E−01 | 9.42E−01 | 6.44E−01 |
| HALLMARK_NOTCH_SIGNALING | 32 | 26 | 0.032 | 0.040 | 9.04E−01 | 9.13E−01 | 9.42E−01 | 9.44E−01 |
| HALLMARK_COAGULATION | 138 | 84 | −0.023 | 0.001 | 9.31E−01 | 5.45E−01 | 9.50E−01 | 6.99E−01 |
| HALLMARK_ANDROGEN_RESPONSE | 100 | 87 | 0.025 | 0.062 | 9.72E−01 | 7.61E−01 | 9.72E−01 | 8.45E−01 |

TABLE 4
Clade-specific transcriptional changes and cell cytotoxicity observed at 24 hours.

| | Set. | Genes. | S149.v.64_ | S149.v.64_ | S149.v.64_ |
|---|---|---|---|---|---|
| MSigDB.Gene.Set | Size | Expressed | 8 hr.avg.logFC | 8 hr.PValue | 8 hr.FDR |
| HALLMARK_TNFA_SIGNALING_VIA_NFKB | 200 | 148 | 0.454 | 1.45E−17 | 7.26E−16 |
| HALLMARK_UNFOLDED_PROTEIN_RESPONSE | 113 | 107 | 0.222 | 9.68E−09 | 2.42E−07 |
| HALLMARK_MYC_TARGETS_V2 | 58 | 57 | 0.267 | 7.13E−08 | 1.19E−06 |
| HALLMARK_OXIDATIVE_PHOSPHORYLATION | 200 | 195 | −0.108 | 2.91E−07 | 3.63E−06 |
| HALLMARK_TGF_BETA_SIGNALING | 54 | 48 | 0.298 | 1.53E−04 | 1.53E−03 |
| HALLMARK_INFLAMMATORY_RESPONSE | 200 | 91 | 0.216 | 3.05E−04 | 2.55E−03 |
| HALLMARK_FATTY_ACID_METABOLISM | 158 | 130 | −0.108 | 6.64E−04 | 4.75E−03 |
| HALLMARK_IL2_STAT5_SIGNALING | 199 | 133 | 0.151 | 7.72E−04 | 4.83E−03 |
| HALLMARK_IL6_JAK_STAT3_SIGNALING | 87 | 47 | 0.246 | 1.13E−03 | 6.27E−03 |
| HALLMARK_UV_RESPONSE_DN | 144 | 114 | 0.150 | 2.56E−03 | 1.28E−02 |
| HALLMARK_APOPTOSIS | 161 | 125 | 0.156 | 6.41E−03 | 2.91E−02 |
| HALLMARK_PEROXISOME | 104 | 88 | −0.080 | 1.65E−02 | 6.66E−02 |
| HALLMARK_ADIPOGENESIS | 200 | 171 | 0.061 | 1.73E−02 | 6.66E−02 |
| HALLMARK_REACTIVE_OXYGEN_SPECIES_PATHWAY | 49 | 42 | −0.012 | 1.94E−02 | 6.94E−02 |
| HALLMARK_MITOTIC_SPINDLE | 199 | 195 | 0.098 | 2.24E−02 | 7.15E−02 |
| HALLMARK_KRAS_SIGNALING_UP | 200 | 96 | 0.174 | 2.29E−02 | 7.15E−02 |
| HALLMARK_P53_PATHWAY | 200 | 170 | 0.139 | 2.72E−02 | 7.99E−02 |
| HALLMARK_XENOBIOTIC_METABOLISM | 200 | 147 | −0.062 | 4.25E−02 | 1.16E−01 |
| HALLMARK_EPITHELIAL_MESENCHYMAL_TRANSITION | 200 | 114 | 0.115 | 4.40E−02 | 1.16E−01 |
| HALLMARK_BILE_ACID_METABOLISM | 112 | 73 | −0.061 | 4.79E−02 | 1.20E−01 |
| HALLMARK_WNT_BETA_CATENIN_SIGNALING | 42 | 31 | 0.140 | 9.59E−02 | 2.28E−01 |
| HALLMARK_UV_RESPONSE_UP | 158 | 129 | 0.062 | 1.23E−01 | 2.80E−01 |
| HALLMARK_KRAS_SIGNALING_DN | 200 | 68 | −0.117 | 1.60E−01 | 3.47E−01 |
| HALLMARK_GLYCOLYSIS | 200 | 163 | −0.025 | 2.03E−01 | 4.03E−01 |
| HALLMARK_PANCREAS_BETA_CELLS | 40 | 15 | −0.098 | 2.11E−01 | 4.03E−01 |
| HALLMARK_PI3K_AKT_MTOR_SIGNALING | 105 | 88 | 0.061 | 2.18E−01 | 4.03E−01 |
| HALLMARK_DNA_REPAIR | 150 | 141 | −0.038 | 2.86E−01 | 5.07E−01 |
| HALLMARK_APICAL_JUNCTION | 200 | 125 | 0.051 | 3.13E−01 | 5.07E−01 |
| HALLMARK_APICAL_SURFACE | 44 | 22 | −0.112 | 3.16E−01 | 5.07E−01 |
| HALLMARK_PROTEIN_SECRETION | 96 | 92 | −0.038 | 3.17E−01 | 5.07E−01 |
| HALLMARK_ESTROGEN_RESPONSE_LATE | 200 | 152 | −0.016 | 3.24E−01 | 5.07E−01 |
| HALLMARK_HEDGEHOG_SIGNALING | 36 | 22 | 0.096 | 3.43E−01 | 5.19E−01 |
| HALLMARK_HEME_METABOLISM | 200 | 153 | 0.041 | 3.69E−01 | 5.43E−01 |
| HALLMARK_ALLOGRAFT_REJECTION | 200 | 86 | 0.032 | 4.03E−01 | 5.64E−01 |
| HALLMARK_G2M_CHECKPOINT | 200 | 196 | 0.040 | 4.06E−01 | 5.64E−01 |
| HALLMARK_CHOLESTEROL_HOMEOSTASIS | 74 | 64 | 0.008 | 4.42E−01 | 5.98E−01 |
| HALLMARK_ANGIOGENESIS | 36 | 24 | 0.088 | 5.09E−01 | 6.69E−01 |
| HALLMARK_COAGULATION | 138 | 84 | 0.024 | 5.25E−01 | 6.73E−01 |
| HALLMARK_INTERFERON_GAMMA_RESPONSE | 200 | 116 | −0.005 | 5.46E−01 | 6.82E−01 |
| HALLMARK_INTERFERON_ALPHA_RESPONSE | 97 | 64 | −0.065 | 6.00E−01 | 7.31E−01 |
| HALLMARK_COMPLEMENT | 200 | 129 | 0.044 | 6.84E−01 | 8.01E−01 |
| HALLMARK_ANDROGEN_RESPONSE | 100 | 87 | 0.037 | 6.89E−01 | 8.01E−01 |
| HALLMARK_MYC_TARGETS_V1 | 200 | 199 | 0.005 | 7.17E−01 | 8.14E−01 |
| HALLMARK_NOTCH_SIGNALING | 32 | 26 | 0.008 | 7.75E−01 | 8.59E−01 |
| HALLMARK_MTORC1_SIGNALING | 200 | 189 | 0.018 | 7.93E−01 | 8.59E−01 |
| HALLMARK_HYPOXIA | 200 | 150 | 0.015 | 8.07E−01 | 8.59E−01 |
| HALLMARK_E2F_TARGETS | 200 | 200 | 0.008 | 8.25E−01 | 8.59E−01 |
| HALLMARK_MYOGENESIS | 200 | 100 | −0.007 | 8.57E−01 | 8.59E−01 |
| HALLMARK_SPERMATOGENESIS | 135 | 63 | −0.006 | 8.59E−01 | 8.59E−01 |
| HALLMARK_ESTROGEN_RESPONSE_EARLY | 200 | 156 | 0.059 | 1.81E−01 | 3.78E−01 |

### Example 3—Detection of Biotherapeutic Strains

Detection and differentiation of bacterial strains of the invention provides the capacity to target beneficial therapeutic interventions, both conventional and microbiome based, to best suit patient needs. This capacity has the potential for use as a companion diagnostic for stratification of, for example, IBD patients. Multiple methods could be applied to achieve this differentiation including DNA, RNA, metabolic signature and protein-based assays. The following data demonstrates the implementation of this method for specific detection using a DNA and qPCR-based implementation.

Methods qPCR was performed on a QuantStudio™ 6 Flex Real-Time PCR System using the following primers:

Non-Inflammatory (non-cytotoxic) strains
Forward:
(SEQ ID NO: 75)
CGAGGTACACCGTAAGTTGTTGCT
Reverse:
(SEQ ID NO: 76)
ACCAGGACACGTAGACTTCACAGT
Inflammatory (cytotoxic) strains
Forward:
(SEQ ID NO: 83)
CCTTTACCATGGCCGTGTAGC
Reverse:
(SEQ ID NO: 84)
CCATGAATGGTGTTCCTCCTTCTC

The DNA was diluted 1:5 in DEPC-treated water (Applied Biosystems). Applied Biosystems SYBR Magic Master Mix (Applied Biosystems; Foster City, California, USA) was used in total reaction volumes of 10 μl. Each reaction contained 5 μl of the SYBR Magic Master Mix, 0.2 μl each of the appropriate forward and reverse primers (final concentration 0.2 mM each), 2 μl of DNA, and 2.6 μl of DEPC water. The samples were loaded in triplicate into MicroAmp Optical 384-well Reaction plates (Applied Biosystems), sealed with a MicroAmp Optical adhesive cover. A no-template negative control was included on all plates.

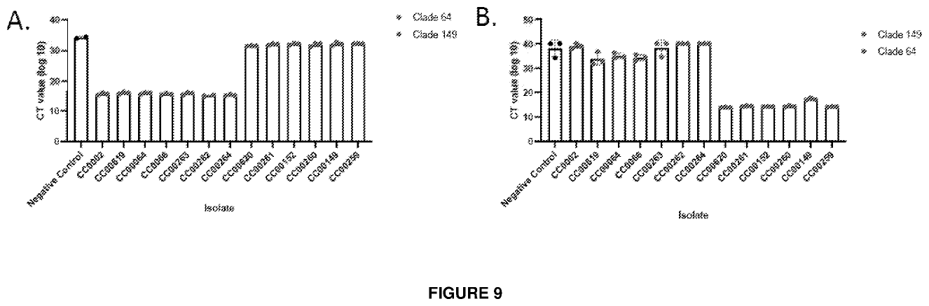

Both primer sets were found to be clade specific, distinguishing between non-inflammatory strains and inflammatory strains of the invention (FIG. 9).

### Example 4—Comparison of Sequence Identities

The strain of the invention, comprising a 16s ribosomal RNA (rRNA) gene having a nucleotide sequence as shown in any one of SEQ ID NO's 1 to 74 (and, in particular, SEQ ID NO 45), was compared to SEQ ID NO's 89 to 217 using sequence identity analysis.

In a preferred embodiment of the invention, the strain of the invention comprising a 16s ribosomal RNA (rRNA) gene having a nucleotide sequence as shown in any one of SEQ ID NO's 1 to 74 does not comprise any one of SEQ ID NO's 89 to 217 presented in Table 5. As discussed above under the preferred embodiments, in a further embodiment, the strain of, or for use in, the invention comprises a 16s ribosomal RNA (rRNA) gene having a nucleotide sequence as shown in any one of SEQ ID NO's 1 to 74, or a nucleotide sequence at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8% or at least 99.9% identical to one or more of SEQ ID NO's 1 to 74, and wherein the 16s ribosomal RNA (rRNA) gene does not comprise any one of SEQ ID NO's 89 to 217. In an alternative embodiment, the 16s ribosomal RNA (rRNA) gene does comprise any one of SEQ ID NO's 89 to 217.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Buchfink et al. Nature Methods 18:366-368.
Cao et al. (2009) BMC Bioinformatics 10:34.
Clausen et al. (2018) BMC Bioinformatics 19:307.
Golińska et al. (2013) World Journal of Gastroenterology 19:3562-3572.
Graham and Xavier (2020) Nature 578:527-539.
Forster et al. (2019) Nat. Biotechnol. 37:186-192.
Liberzon et al. (2015) Cell Syst. 1:417-425.
Liu et al (2019) Nucleic Acids Res. 47 (D1): D687-D692
Marcelino et al. (2020) Genome Biology 21:103.
Needleman and Wunsch (1970) J. Mol Biol. 45:443-453.
Ni et al. (2017) Nat. Rev. Gastroenterol. Hepatol. 14:573-584.
O'Leary et al. (2016) Nucleic Acids Research 44: D733-D745.
Page et al. (2015) Bioinformatics 31:3691-3693.
Parks et al. (2014) Genome Research 25:1043-1055.
Rohart et al. (2017) PLOS Computational Biology 13: e1005752.
Schirmer et al. (2019) Nat. Rev. Microbiol. 17:497-511.
Seemann (2014) Bioinformatics 30:2068-9.
Stamatakis (2014) Bioinformatics 30:1312-1313.

### NUCLEOTIDE SEQUENCES

TABLE 5
PatentIn Field Identifier and Field Name Key

| Field Identifier | Field Name |
|---|---|
| N/A | Project Name |
| <110> | Applicant Name |
| <120> | Title of Invention |
| <130> | File Reference |
| <140> | Current Application Number |
| <141> | Current Filing Date |
| <150> | Earlier Application Number |
| <151> | Earlier Filing Date |
| N/A | Sequence File Name |
| <160> | Number of Sequences |
| <170> | Software |
| <210> | Information for SEQ ID No. |
| <211> | Length |
| <212> | Type |
| <213> | Organism |
| <220> | Feature |
| <221> | Name/key |
| <222> | Location |
| <223> | Other Information |
| <300> | Publication Information |
| <301> | Authors |
| <302> | Title (of Publication) |
| <303> | Journal (name) |
| <304> | Volume |
| <305> | Issue |
| <306> | Pages |
| <307> | Date |
| <308> | Database Accession Number |
| <309> | Database Entry Date |
| <310> | Document Number |
| <311> | Filing Date |
| <312> | Publication Date |
| <313> | Relevant Residues |
| <400> | Sequence Description |

1. A method of reducing or preventing gastrointestinal tract mucosal inflammation in a subject, the method comprising administering to the subject a therapeutically effective amount of a biotherapeutic composition comprising a non-inflammatory strain of Enterococcus sp.

2. The method of claim 1, wherein the strain comprises a 16s ribosomal RNA (rRNA) gene having a nucleotide sequence as shown in any one of SEQ ID NO's 1 to 44, or a nucleotide sequence with at least 99%, at least 97% or at least 95% sequence identity to one or more of SEQ ID NO's 1 to 44.

3. A method of treating or preventing a dysbiosis of the gastrointestinal tract in a subject, the method comprising administering to the subject a therapeutically effective amount of a biotherapeutic composition comprising a strain of Enterococcus sp. which comprises a 16s ribosomal RNA (rRNA) gene having a nucleotide sequence as shown in any one of SEQ ID NO's 1 to 74, or a nucleotide sequence with at least 99%, at least 97% or at least 95% sequence identity to one or more of SEQ ID NO's 1 to 74.

4. The method of claim 3, wherein the Enterococcus sp. is a non-inflammatory strain and comprises a 16s ribosomal RNA (rRNA) gene having a nucleotide sequence as shown in any one of SEQ ID NO's 1 to 44, or a nucleotide sequence with at least 99%, at least 97% or at least 95% sequence identity to one or more of SEQ ID NO's 1 to 44.

5. The method of claim 3, wherein the Enterococcus sp, is an inflammatory strain and comprises a 16s ribosomal RNA (rRNA) gene having a nucleotide sequence as shown in any one of SEQ ID NO's 45 to 74, or a nucleotide sequence with at least 99%, at least 97% or at least 95% sequence identity to one or more of SEQ ID NO's 45 to 74.

6. The method of claim 3, wherein the Enterococcus sp, is an inflammatory strain and comprises a 16s ribosomal RNA (rRNA) gene having a nucleotide sequence as shown in any one of SEQ ID NO's 45 to 50, or a nucleotide sequence with at least 99%, at least 97% or at least 95% sequence identity to one or more of SEQ ID NO's 45 to 50.

7. The method of claim 3, wherein the Enterococcus sp, is an inflammatory strain and comprises a 16s ribosomal RNA (rRNA) gene having a nucleotide sequence as shown in any one of SEQ ID NO's 1 to 74, or a nucleotide sequence with at least 99%, at least 97% or at least 95% sequence identity to one or more of SEQ ID NO's 1 to 74 and wherein the 16s ribosomal RNA (rRNA) gene having a nucleotide sequence or a nucleotide sequence with at least 99%, at least 97% or at least 95% sequence identity to one or more of SEQ ID NO's 1 to 74 does not comprise a nucleotide sequence as shown in any one of SEQ ID NO's 89 to 217.

8. The method according to claim 1, wherein the dysbiosis and/or inflammation is associated with one or more of inflammatory bowel disease (IBD), pouchitis, irritable bowel syndrome (IBS), an enteric bacterial infection, a metabolic disease, a neuropsychiatric disorder, an autoimmune disease, an allergic disorder, hepatic encephalopathy, or a cancer.

9. The method of claim 8, wherein the IBD is ulcerative colitis (UC) or Crohn's disease.

10. The method according to any one of claims 1 to 9 claim 1, wherein the composition further comprises a prebiotic.

11. The method according to any one of claims 1 to 10 claim 1, wherein the composition further comprises a carrier.

12. The method according to any one of claims 1 to 11 claim 1, wherein the composition further comprises insoluble fiber, a buffer, an osmotic agent, an antifoaming agent, and/or a preservative.

13. The method according to any one of claims 1 to 12 claim 1, wherein the composition comprises a chemostat medium.

14. The method according to any one of claims 1 to 13 claim 1, wherein the composition which is a saline composition.

15. The method according to any one of claims 1 to 14 claim 1, wherein the composition is administered orally or rectally.

16. The method according to any one of claims 1 to 15 claim 1, wherein the composition further comprises a stabiliser and/or a cryoprotectant.

17. The method according to any one of claims 1 to 16 claim 1, wherein the composition is the form of a capsule, a tablet, or an enema.

18. The method of claim 17, wherein the capsule or tablet is enteric-coated, pH dependant, slow-release, and/or gastro-resistant.

19. The method according to any one of claims 1 to 18 claim 1, wherein the subject is a human.

20. (canceled)

21. An isolated strain of Enterococcus sp. which comprises a 16s ribosomal RNA (rRNA) gene having a nucleotide sequence as shown in any one of SEQ ID NO's 1 to 74, or a nucleotide sequence with at least 99%, at least 97% or at least 95% sequence identity to one or more of SEQ ID NO's 1 to 74.

22.-34. (canceled)


SEQUENCE LISTING (217 sequences)

SEQ ID NO: 1 - DNA, 1179 bp, Enterococcus sp.
  source 1..1179: mol_type = genomic DNA; note = isolate CC00149
ctatacatgcagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgcccc

SEQ ID NO: 2 - DNA, 1159 bp, Enterococcus sp.
  source 1..1159: mol_type = genomic DNA; note = isolate CC00259
ctatacatgcagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatg

SEQ ID NO: 3 - DNA, 1214 bp, Enterococcus sp.
  source 1..1214: mol_type = genomic DNA; note = isolate CC00261
atacatgcagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgaccggggctacccacgtgctacaatgggaagt

SEQ ID NO: 4 - DNA, 1192 bp, Enterococcus sp.
  source 1..1192: mol_type = genomic DNA; note = isolate CC00260
ctatacatgcagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgaccggggc

SEQ ID NO: 5 - DNA, 1179 bp, Enterococcus sp.
  source 1..1179: mol_type = genomic DNA; note = isolate CC00620
acatgcagtcgtacgcttgctattttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggggggggatgacggccagatcatcatgcgcc

SEQ ID NO: 6 - DNA, 1186 bp, Enterococcus sp.
  source 1..1186: mol_type = genomic DNA; note = isolate CC00152
tgcagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgaccggggct

SEQ ID NO: 7 - DNA, 1112 bp, Enterococcus sp.
  source 1..1112: mol_type = genomic DNA; note = isolate mc056tix_3d_mc056_57_d3
ctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaagctcgaaagcgtgggggagcaaacaggattagataccctgggtagtccacgcccgtaaacgatgaagtgctaagtgttggaagggtttccgccccttcagtgctgcaggctaacgcatttaagcactccgccctgggggagtacgaaccgccaaggttgaaaactcaaaaggaaattgaccggggggcccgcaccaagcgggtggaagcatgtgggtttaatttcgaagccaacgccgaagaaacctttaccagggtcttggacatcccttttgaaccactcctagaagataggaagcttccccccttccggggggcaaaagtgaacaaggtgggtggcatgggtttgtccgtccagcctccgtgtccctggagaatgtttgg

SEQ ID NO: 8 - DNA, 1102 bp, Enterococcus sp.
  source 1..1102: mol_type = genomic DNA; note = isolate mc022rnx_e1
atacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttggggttaagtcccgcaacgaagcgcaacccttattggttagttgccat

SEQ ID NO: 9 - DNA, 1178 bp, Enterococcus sp.
  source 1..1178: mol_type = genomic DNA; note = isolate mc056tix_3h_mc056_57_h3
ctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatggcc

SEQ ID NO: 10 - DNA, 1082 bp, Enterococcus sp.
  source 1..1082: mol_type = genomic DNA; note = isolate mc056tix_3f_mc056_57_f3
ctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacggggggcccgcacaagcggtggagcatgtggtttaattcgaagcaacgcgaagaaaccttaccaggtcttgacatcctttgaccactctagagatagagcttccccttcgggggcaaagtgacaagtggttgcatgggttgtcgtcagcctcgtgtcctgaaaatgttggggttaagtccccgcaacg

SEQ ID NO: 11 - DNA, 988 bp, Enterococcus sp.
  misc_feature 907: note = n is a, c, g, or t
  misc_feature 923: note = n is a, c, g, or t
  misc_feature 928: note = n is a, c, g, or t
  misc_feature 936: note = n is a, c, g, or t
  misc_feature 939: note = n is a, c, g, or t
  misc_feature 947..949: note = n is a, c, g, or t
  misc_feature 965: note = n is a, c, g, or t
  misc_feature 967: note = n is a, c, g, or t
  misc_feature 975: note = n is a, c, g, or t
  misc_feature 979: note = n is a, c, g, or t
  source 1..988: mol_type = genomic DNA; note = isolate mc036cna_f03
agtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggancatgtggtttaattcnaagcnacgcgaanaanccttaccnnncttgacatcctttgancnctctaganatanagcttcccc

SEQ ID NO: 12 - DNA, 1184 bp, Enterococcus sp.
  source 1..1184: mol_type = genomic DNA; note = isolate mc056tix_4a_mc056_57_a4
ggcgtgctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcatgagatgttgggttaagtcccgcacgagcgcaacccttattgttagttgccatcatttagttgggcactctagcaggactgccggtgacaaaccggaggaagttggggatgacgtcaaatcatcatgcccc

SEQ ID NO: 13 - DNA, 1119 bp, Enterococcus sp.
  source 1..1119: mol_type = genomic DNA; note = isolate mc056tix_3e_mc056_57_e3
ctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtgggtgcagggttgtccgtcacctcctgtcctgagatgttggggttaagtcccggcaacgaaccgcaacccttattggttagttgccatcattttagttgg

SEQ ID NO: 14 - DNA, 1191 bp, Enterococcus sp.
  source 1..1191: mol_type = genomic DNA; note = isolate mc056cix_8d_mc056_57_d8
ctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtacacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcctgagatgttgggttaagtcccgcaacgagcgcaacccttattgttagttgccatcatttagttgggcactctagcaagactgccggtgacaaaccggaggaaggtggggatgacgtcaaatcatcatgccccttatgaactggg

SEQ ID NO: 15 - DNA, 1208 bp, Enterococcus sp.
  source 1..1208: mol_type = genomic DNA; note = isolate mc056cix_8e_mc056_57_e8
ctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcctgagatgttgggttaagtcccgcaacaagcgcaacccttattgttagttgccatcatttagttgggcactctagcaggactgcccgtgaaaaaaccggaggaagggtggggatgaacgtcaaatcatcatgcccccttatgaactggggctaacaacctgg

SEQ ID NO: 16 - DNA, 1157 bp, Enterococcus sp.
  source 1..1157: mol_type = genomic DNA; note = isolate mc056tix_3a_mc056_57_a3
ctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcagggttgtcgtcagctcgtgtcgtgaaatgttgggttaagtcccgcaacaagcgcaacccttatggttagttgccatcatttagttgggcattctagcaagactgcccggtgacaaaccggaaggaaggtggg

SEQ ID NO: 17 - DNA, 1129 bp, Enterococcus sp.
  source 1..1129: mol_type = genomic DNA; note = isolate mc056tix_1c_mc056_57_c1
ctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctgggggagtacgaccgcaaggttgaaactcaaaggaattgacggggggcccgcacaaccggtggagcatgtggtttaattcgaagcaacgcgaagaaaccttaccagggtcttgacatccctttgaccactctagagatagaagcttcccccttcgggggcaaagtgaccaggtggttgcatgggttgtcgtcaacctcgtgtccgtgaaaatgttggggttaaattcccgcaaacaagcgcaaacctttattggttaattggccatccatttaat

SEQ ID NO: 18 - DNA, 1167 bp, Enterococcus sp.
  source 1..1167: mol_type = genomic DNA; note = isolate mc056tix_3b_mc056_57_b3
ctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacggggggcccgcacaagcggtggagcatgtggtttaattcgaagcaacgcgaagaaccttaccaggtcttgacatcctttgaccactctagagatagagcttcccccttcgggggcaaagtgacaagggggtgcatggtttgtcgtcagcttcgtgtcctggagatgttggggttaagtcccgcaaccaagcgccacccctaattgttaatttgccatcattttagttggggcacttctagccaggactggccgggtgacaaaaccggaaggaa

SEQ ID NO: 19 - DNA, 998 bp, Enterococcus sp.
  misc_feature 908: note = n is a, c, g, or t
  misc_feature 937: note = n is a, c, g, or t
  misc_feature 966: note = n is a, c, g, or t
  misc_feature 974: note = n is a, c, g, or t
  misc_feature 976: note = n is a, c, g, or t
  misc_feature 980: note = n is a, c, g, or t
  source 1..998: mol_type = genomic DNA; note = isolate mc036rna2_h03
aagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggancatgtggtttaattcgaagcaacgcgaanaaccttaccaggtcttgacatcctttgancactctananatanagcttccccttcgggggc

SEQ ID NO: 20 - DNA, 1163 bp, Enterococcus sp.
  source 1..1163: mol_type = genomic DNA; note = isolate mc056cix_5f_mc056_57_f5
ctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcctgaaatgttgggttaagtcccgcaacgagcgcaacccttattgttagttgccatcatttaagtggggcactctagcaagactgcccggtgacaaaccggaaggaaggtgggggatg

SEQ ID NO: 21 - DNA, 1204 bp, Enterococcus sp.
  source 1..1204: mol_type = genomic DNA; note = isolate mc056cix_6d_mc056_57_d6
ctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttggggactctagcaagactgccggtggacaaaccggaggaaggtggggatgacgtcaaatcatcatgccccttatgacctgggctaccaccgtgc

SEQ ID NO: 22 - DNA, 1183 bp, Enterococcus sp.
  source 1..1183: mol_type = genomic DNA; note = isolate mc056tix_3g_mc056_57_g3
tgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgg

SEQ ID NO: 23 - DNA, 1175 bp, Enterococcus sp.
  source 1..1175: mol_type = genomic DNA; note = isolate mc056cix_8c_mc056_57_c8
acatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtgggggatgacgtcaaatcatcatgccc

SEQ ID NO: 24 - DNA, 938 bp, Enterococcus sp.
  source 1..938: mol_type = genomic DNA; note = isolate mc056cix_7h_mc056_57_h7
ctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaggaattgacgggggcccgcacagcggtggagcatgtggtttattcgaagcaacg

SEQ ID NO: 25 - DNA, 1194 bp, Enterococcus sp.
  source 1..1194: mol_type = genomic DNA; note = isolate mc056tix_2b_mc056_57_b2
gcgtgctatacatgcagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgaaatgttgggttaagtcccgcaacgagcgcaacccttattgttagttgccatcatttagttgggcactctagcaagactgccggtgacaaaccggaggaaggtggggatgacgtcaaatcatcatgccccttatgactgg

SEQ ID NO: 26 - DNA, 1137 bp, Enterococcus sp.
  source 1..1137: mol_type = genomic DNA; note = isolate mc017tn_x_b6
tatacatgcagtcgtacgcttgctttttctcaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttggggttaagtccccgcaacgaagcgcaaccctttattgttagttgccatcatttcattggggcactctagcaagactggc

SEQ ID NO: 27 - DNA, 1087 bp, Enterococcus sp.
  source 1..1087: mol_type = genomic DNA; note = isolate mc017cn_x_d3
ctataatgcaagtcgtacgctgtgctatttgctaagcggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaattcccgcaacgagcgcaacc

SEQ ID NO: 28 - DNA, 1146 bp, Enterococcus sp.
  source 1..1146: mol_type = genomic DNA; note = isolate mc017cn_x_d2
ctatacatgcaagtcgtacgctgtgctatttgctcaccaggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctgggggagtacgaccgcaaggtttgaaactcaaaagaaattgacggggggccccgcacaagccggtggaagcatgtgggtttaattcgaaaccaacgcgaaagaacctttaccagggtcttggacattcctttggaccacttctagaagatagaagctttccccctttcgggggggcaaaagtggacaaggtgggtggcatgggttggtccatccaacttcggtgttcgtgaagaatgtttggggtttaaagtccccgtcaaccaaggcgccaaccccttaattggt

SEQ ID NO: 29 - DNA, 1017 bp, Enterococcus sp.
  misc_feature 136: note = n is a, c, g, or t
  misc_feature 772: note = n is a, c, g, or t
  misc_feature 877: note = n is a, c, g, or t
  misc_feature 905: note = n is a, c, g, or t
  misc_feature 921: note = n is a, c, g, or t
  misc_feature 924: note = n is a, c, g, or t
  misc_feature 926: note = n is a, c, g, or t
  misc_feature 931: note = n is a, c, g, or t
  misc_feature 944: note = n is a, c, g, or t
  misc_feature 954: note = n is a, c, g, or t
  misc_feature 973..974: note = n is a, c, g, or t
  misc_feature 977: note = n is a, c, g, or t
  misc_feature 986: note = n is a, c, g, or t
  misc_feature 990: note = n is a, c, g, or t
  misc_feature 995: note = n is a, c, g, or t
  misc_feature 997: note = n is a, c, g, or t
  misc_feature 999: note = n is a, c, g, or t
  misc_feature 1001: note = n is a, c, g, or t
  misc_feature 1003..1005: note = n is a, c, g, or t
  misc_feature 1008: note = n is a, c, g, or t
  misc_feature 1011..1012: note = n is a, c, g, or t
  source 1..1017: mol_type = genomic DNA; note = isolate mc026cna2_e06
tcgaacgcttctttttccaccggagcttgctccaccggaaaaagaggagtggcgaacgggtgagtaacacgtgggtaacctgcccatcagaaggggataacacttggaaacaggtgctaataccgtataacaatcnaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtanacgatgagtgctaagtgttggagggtttccgcccttcagtgctgcagctaacgcattaagcactccgcctggggagtacgaccgcaaggttgaaactcaaagganttgacgggggcccgcacaagcggtggancatgtggtttaattcnaancnacgcnaagaaccttaccnggtcttgacntcctttgaccactctaganntanagcttcccnttcnggggncnangngnnnggnggnngcatg

SEQ ID NO: 30 - DNA, 987 bp, Enterococcus sp.
  misc_feature 136: note = n is a, c, g, or t
  misc_feature 717: note = n is a, c, g, or t
  misc_feature 845: note = n is a, c, g, or t
  misc_feature 852: note = n is a, c, g, or t
  misc_feature 858: note = n is a, c, g, or t
  misc_feature 905: note = n is a, c, g, or t
  misc_feature 915: note = n is a, c, g, or t
  misc_feature 918..919: note = n is a, c, g, or t
  misc_feature 921: note = n is a, c, g, or t
  misc_feature 926: note = n is a, c, g, or t
  misc_feature 934: note = n is a, c, g, or t
  misc_feature 937: note = n is a, c, g, or t
  misc_feature 944: note = n is a, c, g, or t
  misc_feature 954: note = n is a, c, g, or t
  misc_feature 971: note = n is a, c, g, or t
  misc_feature 973: note = n is a, c, g, or t
  misc_feature 977..978: note = n is a, c, g, or t
  misc_feature 982: note = n is a, c, g, or t
  source 1..987: mol_type = genomic DNA; note = isolate mc026rna2_g06
tcgaacgcttctttttccaccggagcttgctccaccggaaaaagaggagtggcgaacgggtgagtaacacgtgggtaacctgcccatcagaaggggataacacttggaaacaggtgctaataccgtataacaatcnaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgangctcgaaagcgtggggagcaaacaggattagataccctggtagtccacgccgtaaacgatgagtgctaagtgttggagggtttccgcccttcagtgctgcagctaacgcattaagcactccgcctggngagtacnaccgcnaggttgaaactcaaaggaattgacgggggcccgcacaagcggtggancatgtggttnaanncnaagcnacgcgaanaancttaccnggtcttgacntcctttgaccactctananatanngctncccct

SEQ ID NO: 31 - DNA, 1185 bp, Enterococcus sp.
  source 1..1185: mol_type = genomic DNA; note = isolate mc059rn_fx_g10
ctatacatgcagtcgtacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaataaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatga

SEQ ID NO: 32 - DNA, 1075 bp, Enterococcus sp.
  source 1..1075: mol_type = genomic DNA; note = isolate mc017rn_x_f4
tgcagttcgtacgcttgctattttgctccagcggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaagtggtgcatggttgtcgtcagctcgtgtcgtgagatgttgggttaagtcccgcaacgag

SEQ ID NO: 33 - DNA, 1000 bp, Enterococcus sp.
  misc_feature 448: note = n is a, c, g, or t
  misc_feature 907: note = n is a, c, g, or t
  misc_feature 923: note = n is a, c, g, or t
  misc_feature 936: note = n is a, c, g, or t
  misc_feature 956: note = n is a, c, g, or t
  source 1..1000: mol_type = genomic DNA; note = isolate mc017rn_12f_a
agtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatntaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggancatgtggtttaattcnaagcaacgcgaanaaccttaccaggtcttgacntcctttgaccactctagagatagagcttccccttcgggggcaaa

SEQ ID NO: 34 - DNA, 998 bp, Enterococcus sp.
  misc_feature 446: note = n is a, c, g, or t
  misc_feature 871..873: note = n is a, c, g, or t
  misc_feature 905: note = n is a, c, g, or t
  misc_feature 945: note = n is a, c, g, or t
  misc_feature 990: note = n is a, c, g, or t
  source 1..998: mol_type = genomic DNA; note = isolate mc017tn_12a_ma
tcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatntaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcnnngaattgacggggggcccgcacaagcggtggancatgtggtttaattcgaagcaacgcgaagaaccttaccangtcttgacatcctttgaccactctagagatagagcttccccttcnggggcaaa

SEQ ID NO: 35 - DNA, 1003 bp, Enterococcus sp.
  misc_feature 206: note = n is a, c, g, or t
  misc_feature 905: note = n is a, c, g, or t
  misc_feature 913: note = n is a, c, g, or t
  misc_feature 921: note = n is a, c, g, or t
  misc_feature 926: note = n is a, c, g, or t
  misc_feature 973: note = n is a, c, g, or t
  misc_feature 982: note = n is a, c, g, or t
  misc_feature 995: note = n is a, c, g, or t
  source 1..1003: mol_type = genomic DNA; note = isolate mc017tn_1a_ma
tcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagcnagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggancatgtggnttaattcnaagcnacgcgaagaaccttaccaggtcttgacatcctttgaccactctaganatagagctnccccttcgggggnaaagtgac

SEQ ID NO: 36 - DNA, 999 bp, Enterococcus sp.
  misc_feature 21: note = n is a, c, g, or t
  misc_feature 448: note = n is a, c, g, or t
  misc_feature 879: note = n is a, c, g, or t
  misc_feature 946: note = n is a, c, g, or t
  misc_feature 948: note = n is a, c, g, or t
  misc_feature 956: note = n is a, c, g, or t
  misc_feature 975: note = n is a, c, g, or t
  misc_feature 992: note = n is a, c, g, or t
  source 1..999: mol_type = genomic DNA; note = isolate mc017rn_12e_ma
agtcgtacgcttctttttccnccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatntaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaagganttgacgggggcccgcacaagcggtggagcatgtggtttaattcgaagcaacgcgaagaaccttaccngntcttgacntcctttgaccactctaganatagagcttccccttcnggggcaa

SEQ ID NO: 37 - DNA, 995 bp, Enterococcus sp.
  misc_feature 20: note = n is a, c, g, or t
  misc_feature 447: note = n is a, c, g, or t
  misc_feature 863: note = n is a, c, g, or t
  misc_feature 935: note = n is a, c, g, or t
  misc_feature 945..947: note = n is a, c, g, or t
  misc_feature 950: note = n is a, c, g, or t
  misc_feature 973: note = n is a, c, g, or t
  misc_feature 987: note = n is a, c, g, or t
  source 1..995: mol_type = genomic DNA; note = isolate mc017cn_12c_ma
gtcgtacgcttctttttccnccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatntaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggntgaaactcaaaggaattgacgggggcccgcacaagcggtggagcatgtggtttaattcgaagcaacgcgaanaaccttaccnnnctngacatcctttgaccactctaganatagagcttccccntcgggggc

SEQ ID NO: 38 - DNA, 993 bp, Enterococcus sp.
  misc_feature 18..19: note = n is a, c, g, or t
  misc_feature 136: note = n is a, c, g, or t
  misc_feature 717: note = n is a, c, g, or t
  misc_feature 905: note = n is a, c, g, or t
  misc_feature 944: note = n is a, c, g, or t
  misc_feature 946: note = n is a, c, g, or t
  misc_feature 963: note = n is a, c, g, or t
  misc_feature 973: note = n is a, c, g, or t
  source 1..993: mol_type = genomic DNA; note = isolate mc026rnm2_a08
tcgaacgcttctttttcnnccggagcttgctccaccggaaaaagaggagtggcgaacgggtgagtaacacgtgggtaacctgcccatcagaaggggataacacttggaaacaggtgctaataccgtataacaatcnaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgangctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggancatgtggtttaattcgaagcaacgcgaagaaccttaccngntcttgacatcctttgancactctaganatagagcttccccttcgggg

SEQ ID NO: 39 - DNA, 1067 bp, Enterococcus sp.
  source 1..1067: mol_type = genomic DNA; note = isolate mc017tn_x_a1
ctatacatgcagtcgtacgctgtgctttttctcaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaag

SEQ ID NO: 40 - DNA, 1210 bp, Enterococcus sp.
  source 1..1210: mol_type = genomic DNA; note = isolate mc127rifx_b11
ctatacgtgcagtcgtacgcttcttttgcgcaccggagcttgctccaccggagaaagaagagtggggaacgggtgagtagcacgtggggaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaccggaggaaggggggatgacgtcaaatcatcatgccccttatgacctgggctaccacgggctacatggga

SEQ ID NO: 41 - DNA, 1001 bp, Enterococcus sp.
  misc_feature 21: note = n is a, c, g, or t
  misc_feature 138: note = n is a, c, g, or t
  misc_feature 854: note = n is a, c, g, or t
  misc_feature 907: note = n is a, c, g, or t
  misc_feature 946: note = n is a, c, g, or t
  misc_feature 948: note = n is a, c, g, or t
  misc_feature 975: note = n is a, c, g, or t
  misc_feature 991..992: note = n is a, c, g, or t
  source 1..1001: mol_type = genomic DNA; note = isolate mc026tnm2_c07
agtcgaacgcttctttttccnccggagcttgctccaccggaaaaagaggagtggcgaacgggtgagtaacacgtgggtaacctgcccatcagaaggggataacacttggaaacaggtgctaataccgtataacaatcnaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacnaccgcaaggttgaaactcaaaggaattgacgggggcccgcacaagcggtggancatgtggtttaattcgaagcaacgcgaagaaccttaccngntcttgacatcctttgaccactctaganatagagcttccccttnngggggcaaa

SEQ ID NO: 42 - DNA, 1151 bp, Enterococcus sp.
  source 1..1151: mol_type = genomic DNA; note = isolate mc097cnfx_h6
atacatgcagtcgaacgcttctttttccaccggagcttgctccaccggaaaagaggagtggcgaacgggtgagtaacacgtgggtaacctgcccatcagaaggggataacacttggaaacaggtgctaataccgtataacaatcaaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtggtgggagggtttccgcccttcagtgctgcagctaacgcattaagcactccgcctggggagtacgaccgcaaggttgaaactcaaaggaattgacgggggccgcacaagcggtggagcatgtggtttaattcgaagcaacgcgaagaaccttaccaggtcttgacatcctttgaccactctagagatagagcttccccttcgggggcaagtgacaggtgggggcatggttgtcgtcagctcgtgtcgtgagatgtttgggttaagtcccgcaacgagcgcaacccttattggtagtgccatcattcagttggggactctacaagaatgccgtgacaaccggaggaagggggggg

SEQ ID NO: 43 - DNA, 1149 bp, Enterococcus sp.
  source 1..1149: mol_type = genomic DNA; note = isolate mc127tnfx_c2
agtcgtacgcttgctattttccaccggagcttgcttcccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcacctcctgtcctgaaaatgttgggttaagtcccgcaacgaacgcaacccttattgttagttgccatcattcagttggggactctaccaagactgccggtgacaaaccggaggaaggggg

SEQ ID NO: 44 - DNA, 1114 bp, Enterococcus sp.
  source 1..1114: mol_type = genomic DNA; note = isolate mc127rifx_c10
ctatacgtgcagtcgtacgcttctattttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcctgtcctgaaaatgttgggttaagtcccgcaacgaccgcaacccttattgttagttgccatcattcagttgg

SEQ ID NO: 45 - DNA, 1211 bp, Enterococcus sp.
  source 1..1211: mol_type = genomic DNA; note = isolate CC00064
acatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaccggaggaaggtggggatgacgtcaatcatcatgccccttatgaccgggctaccacgtgctacatgggaagtacac

SEQ ID NO: 46 - DNA, 1176 bp, Enterococcus sp.
  source 1..1176: mol_type = genomic DNA; note = isolate CC00262
ctatacatgcagtcgaacgctttttctttcaccggagcttgctccaccgaagaaaaggagtggcgaacgggtgagtaacacgtgggtaacctgcccatcaaaaggggataacacttggaaacaggtgctaataccgtataacaatagaaaccgcagggtttctatttgaaaggcgcttttgcgtcactgatggatggacccgcggtgcattagctagttggtgaggtaacggctcaccaaggcaacgatgcatagccaacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaccggaggaaggtggggatgacgtcaaatcatcatgcccc

SEQ ID NO: 47 - DNA, 1192 bp, Enterococcus sp.
  source 1..1192: mol_type = genomic DNA; note = isolate CC00264
ctatacatgcagtcgaacgctttttctttcaccggagcttgctccaccgaagaaaaggagtggcgaaggggtgagtaacacgtgggtaacctgcccatcaaaaggggataacacttggaaacaggtgctaataccgtataacaatagaaaccgcagggtttctatttgaaaggcgcttttgcgtcactgatggatggacccgcggtgcattagctagttggtgaggtaacggctcaccaaggcaacgatgcatagccaacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctaaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctggggct

SEQ ID NO: 48 - DNA, 1152 bp, Enterococcus sp.
  source 1..1152: mol_type = genomic DNA; note = isolate CC00263
ctatacatgcagtcgaacgctttttctttcaccggagcttgctccaccgaagaaaaggagtggcgaaggggtgagtaacacgtgggtaacctgcccatcaaaaggggataacacttggaaacaggtgctaataccgtataacaatagaaaccgcagggtttctatttgaaaggcgcttttgcgtcactgatggatggacccgcggtgcattagctagttggtgaggtaacggctcaccaaggcaacgatgcatagccaacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctaaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttatttgggactctagcgagactgccggtgacaaaccgaagaaaggtggg

SEQ ID NO: 49 - DNA, 1103 bp, Enterococcus sp.
  source 1..1103: mol_type = genomic DNA; note = isolate CC00619
ctatacatgcagtcgacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatt

SEQ ID NO: 50 - DNA, 1061 bp, Enterococcus sp.
  source 1..1061: mol_type = genomic DNA; note = isolate CC00002
ctatacatgcagtcgaacgctttttctttcaccggagcttgctccaccgaaagaaaaagagtggcgaacgggtgagtaacacgtgggtaacctgcccatcagaaggggataacacttggaaacaggtgctaataccgtataacactattttccgcatggaagaaagttgaaaggcgcttttgcgtcactgatggatggacccgcggtgcattagctagttggtgaggtaacggctcaccaaggcaacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcgaacaggattagataccctggtagtccacgccgtacacgatgagtgctaagtgttggagggtttccgcccttcagtgctgcagcatacgcattaagcactccgcctggggagtacgaccgcaaggttgagactcaaaggaattgacgggggcccgcacaagcggtggagcatgtggtttatgtcgaagcaacgcgaagaaccttaccaggtcttgacatcctttgaccactctacagatagagcttccccttcgggagcaaagtgacaggtggtgcatgggtgtcttcgactcatgtcgtgaaaggtgggatta

SEQ ID NO: 51 - DNA, 1208 bp, Enterococcus sp.
  source 1..1208: mol_type = genomic DNA; note = isolate CC00066
ctatacatgcagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaccggaggaaggtggggatgacgtcaatcatcatgccccttatgaccgggctaccacgtgctacaatggga

SEQ ID NO: 52 - DNA, 652 bp, Enterococcus sp.
  source 1..652: mol_type = genomic DNA; note = isolate mc071tn_zx_b3
ctatacatgcagtcgaacgctttttctttcaccggagcttgctccaccgaaagaaaaagagtggcgaacgggtgagtaacacgtgggtaacctgcccatcagaaggggataacacttggaaacaggtgctaataccgtataacactattttccgcatggaagaaagttgaaaggcgcttttgcgtcactgatggatggacccgcggtgcattagctagttggtgaggtaacggctcaccaaggcaacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaattctgatgtgaaagccccccggctcaaccggggagggtcattggaaactgggaaaacttgagtggcagaagaggaaaaggggaatttc

SEQ ID NO: 53 - DNA, 695 bp, Enterococcus sp.
  source 1..695: mol_type = genomic DNA; note = isolate mc071cn_fx_e6
ctatacatgcagtcgaacgctttttctttcaccggagcttgctccaccgaaagaaaaagagtggcgaacgggtgagtaacacgtgggtaacctgcccatcagaaggggataacacttggaaacaggtgctaataccgtataacactattttccgcatggaagaaagttgaaaggcgcttttgcgtcactgatggatggacccgcggtgcattagctagttggtgaggtaacggctcaccaaggcaacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgccgaagaggatagtggaattccatgtggatcgttgaaaggggtagatatagggaggaggactccctggt

SEQ ID NO: 54 - DNA, 1104 bp, Enterococcus sp.
  source 1..1104: mol_type = genomic DNA; note = isolate mc071cn_fx_f8
ctatacatgcagtcgaacgctttttctttcaccggagcttgctccaccgaaagaaaaagagtggcgaacgggtgagtaacacgtgggtaacctgcccatcagaaggggataacacttggaaacaggtgctaataccgtataacactattttccgcatggaagaaagttgaaaggcgcttttgcgtcactgatggatggacccgcggtgcattagctagttggtgaggtaacggctcaccaaggcaacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcgaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcacctcctgtcctgaaatgttgggttaagtcccgcaacgagcgcacccttattgttagttgcctccttta

SEQ ID NO: 55 - DNA, 975 bp, Enterococcus sp.
  misc_feature 883: note = n is a, c, g, or t
  misc_feature 912: note = n is a, c, g, or t
  misc_feature 923: note = n is a, c, g, or t
  source 1..975: mol_type = genomic DNA; note = isolate mc001tny_c01
ggagcttgctccaccgaaagaaaaagagtggcgaacgggtgagtaacacgtgggtaacctgcccatcagaaggggataacacttggaaacaggtgctaataccgtataacactattttccgcatggaagaaagttgaaaggcgcttttgcgtcactgatggatggacccgcggtgcattagctagttggtgaggtaacggctcaccaaggcaacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcgaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggancatgtggtttaattcgaagcaacgcgaanaaccttaccangtcttgacatcctttgaccactctagagatagagcttccccttcgggggcaa

SEQ ID NO: 56 - DNA, 831 bp, Enterococcus sp.
  source 1..831: mol_type = genomic DNA; note = isolate mc071tn_zx_d11
ctatacatgcagtcgaacgctttttctttcaccggagcttgctccaccgaaagaaaaagagtggcgaacgggtgagtaacacgtgggtaacctgcccatcagaaggggataacacttggaaacaggtgctaataccgtataacactattttccgcatggaagaaagttgaaaggcgcttttgcgtcactgatggatggacccgcggtgcattagctagttggtgaggtaacggctcaccaaggcaacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcgaacagggattagataccatggtagtccactcccgtaaaccgatgagtgctaattgttggagggtttacgtccctccagggctg

SEQ ID NO: 57 - DNA, 898 bp, Enterococcus sp.
  source 1..898: mol_type = genomic DNA; note = isolate mc071tn_zx_d8
gcgggctatacatgcagtcgaacgctttttctttcaccggagcttgctccaccgaaagaaaaagagtggcgaacgggtgagtaacacgtgggtaacctgcccatcagaaggggataacacttggaaacaggtgctaataccgtataacactattttccgcatggaagaaagttgaaaggcgcttttgcgtcactgatggatggacccgcggtgcattagctagttggtgaggtaacggctcaccaaggcaacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtgcggagcgaacaggattagatactctggtagtccacgccgtacacgatgagtgctatgtgttggagggtttccgctcttcagtgctgcagcacacacattaagcactccgcctggggagtacgaccgccggttgagactcaaaggaattgaca

SEQ ID NO: 58 - DNA, 996 bp, Enterococcus sp.
  misc_feature 19: note = n is a, c, g, or t
  misc_feature 422: note = n is a, c, g, or t
  misc_feature 425: note = n is a, c, g, or t
  misc_feature 904: note = n is a, c, g, or t
  misc_feature 920: note = n is a, c, g, or t
  misc_feature 933: note = n is a, c, g, or t
  misc_feature 972: note = n is a, c, g, or t
  source 1..996: mol_type = genomic DNA; note = isolate mc026tna2_b06
tcgaacgctttttctttcnccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtanaangttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggancatgtggtttaattcnaagcaacgcgaanaaccttaccaggtcttgacatcctttgaccactctaganatagagcttccccttcgggggcaa

SEQ ID NO: 59 - DNA, 808 bp, Enterococcus sp.
  source 1..808: mol_type = genomic DNA; note = isolate mc071tn_zx_c7
ctatacatgcagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaaggcggctctctgggtctgtaactgaacgctgaaggctcgaaaagcgtgggggagcaaacaggaattagaataccctgggtagtccacgcccgtaaaccaatgaagggcta

SEQ ID NO: 60 - DNA, 831 bp, Enterococcus sp.
  source 1..831: mol_type = genomic DNA; note = isolate mc071cn_fx_h7
ctatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactggggaaacttgagtgcagaagaggagagtggaattccatgtgtagcggtgaaatgcgtagatatatggaggaacaccagtggcgaaggcggctctctggtctgtaactgacgctaaggctcgaaagcgtgggaagcaaaccggaattagataccctgggtagtccacgcccgtaaaacgaatgagttgctaaagggttggaagggtttccgccctttc

SEQ ID NO: 61 - DNA, 793 bp, Enterococcus sp.
  source 1..793: mol_type = genomic DNA; note = isolate mc071tn_fx_c5
ctatacatgcaagtcgacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaaaaaggagagtggaattccatgtgtagcggtgaaatgcgtagatatatggaggaacaccagtggcgaaaggcggctctctggtctgtaactgacgctgaagctccaaaagcgtgggggagccaacgggattagataccccggggagtccacgcccgtaaaaca

SEQ ID NO: 62 - DNA, 804 bp, Enterococcus sp.
  source 1..804: mol_type = genomic DNA; note = isolate mc071tn_zx_b4
ctatacatgcagtcgacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactggggaaacttgagtgcagaaaaggagagtggaaattccatgtgtagcggtgaaaatgcgtagatatatggaggaacaccagtggcgaaaggcggctctttggtttgtaaactgaacgcttaaggctccaaaagcgtgggggagccaaaccgggattagaatacccctgggtagtccccgcccgtaaaaccaat

SEQ ID NO: 63 - DNA, 1091 bp, Enterococcus sp.
  source 1..1091: mol_type = genomic DNA; note = isolate mc071tn_fx_d4
ctaatacatgcagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgggagatgttgggttaagtcccgcaacgagggcaacccttatggt

SEQ ID NO: 64 - DNA, 1149 bp, Enterococcus sp.
  source 1..1149: mol_type = genomic DNA; note = isolate mc071tn_fx_d5
catgcagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttaattgggcactctagcgagactgccggtgacaaaccggaggaaaggggg

SEQ ID NO: 65 - DNA, 987 bp, Enterococcus sp.
  misc_feature 423: note = n is a, c, g, or t
  misc_feature 426: note = n is a, c, g, or t
  misc_feature 832: note = n is a, c, g, or t
  misc_feature 860..861: note = n is a, c, g, or t
  misc_feature 916: note = n is a, c, g, or t
  misc_feature 922: note = n is a, c, g, or t
  misc_feature 930: note = n is a, c, g, or t
  misc_feature 932: note = n is a, c, g, or t
  misc_feature 935: note = n is a, c, g, or t
  misc_feature 937: note = n is a, c, g, or t
  misc_feature 939: note = n is a, c, g, or t
  misc_feature 945..947: note = n is a, c, g, or t
  misc_feature 954: note = n is a, c, g, or t
  misc_feature 971: note = n is a, c, g, or t
  misc_feature 973: note = n is a, c, g, or t
  misc_feature 975: note = n is a, c, g, or t
  misc_feature 977..979: note = n is a, c, g, or t
  source 1..987: mol_type = genomic DNA; note = isolate mc026cnm1_e07
gtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtanaangttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaancactccgcctgggggagtacgaccgcanngttgaaactcaaaggaattgacgggggcccgcacaagcggtggagcatgtggttnaattcnaagcaacncnaanancnttaccnnncttgacntcctttgaccactctanananannncttcccct

SEQ ID NO: 66 - DNA, 1081 bp, Enterococcus sp.
  source 1..1081: mol_type = genomic DNA; note = isolate mc071tn_fx_c10
ctaataatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtgggtttaattcgaaagcaacgcgaaagaaccttaccagggtcttgacatccttttgaccactctagaagatagaagcttcccccttcggggggcaaaagtgaccaggtgggtgcatgggttgtccgtccacctccgggtccgggaaaatgttggggtta

SEQ ID NO: 67 - DNA, 1028 bp, Enterococcus sp.
  source 1..1028: mol_type = genomic DNA; note = isolate mc071tn_fx_a9
gcgggcctatacatgcagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcgggtggaacatgtgggtttaattcgaaagcaacgcgaagaaaccttaccagggtcttgacatcctttgaccactctagaaaataaaagcttcccccttcggggggcaaaagtg

SEQ ID NO: 68 - DNA, 802 bp, Enterococcus sp.
  source 1..802: mol_type = genomic DNA; note = isolate mc071tn_fx_a4
ctaatacatgcagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcaaaagaggagagtggaattccatgtgtagcggtggaaatgcgtagatatatggaggaacaccagtgggcgaaaggcggctctctgggtctgtaaactgaacgctgaaggctcgaaaagcgtgggggaagaaaaacagggatttagatacccctgggtaactcccccgccgtaaa

SEQ ID NO: 69 - DNA, 919 bp, Enterococcus sp.
  source 1..919: mol_type = genomic DNA; note = isolate mc071tn_zx_c2
ctatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggaataccaaccgcaagggttgaaactcaagggaattgaaggggggcccccccaaaccggtgg

SEQ ID NO: 70 - DNA, 975 bp, Enterococcus sp.
  source 1..975: mol_type = genomic DNA; note = isolate mc071cn_zx_g1
ctaatacatgcagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctgggggagtaccaaccgcaaggttgaaactcaaaggaattgacggggggcccgccacaagcggtggaagcagggtggtttaaattcgaaagcaaccccgaagaaacctttaccagggtcttg

SEQ ID NO: 71 - DNA, 1074 bp, Enterococcus sp.
  source 1..1074: mol_type = genomic DNA; note = isolate mc071tn_zx_b7
ctatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagccggtggagcatgtggtttaattcgaagcaacgcgaagaaccttaccaggtcttgacatcctttgaccactctagagatagagcttccccttcgggggcaaagtgacaggtggtgcatggttgtcgtcacctccggtcctggaaatgttgggttaagtcccgcaa

SEQ ID NO: 72 - DNA, 1064 bp, Enterococcus sp.
  source 1..1064: mol_type = genomic DNA; note = isolate mc071tn_fx_c1
gcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctgggggagtacgaccgcaaggttgaaactcagaggaattgacgggggcccgcacaagcggtggagcatgtggtttaattcgaaccaacgcgaagaaccttaccaggtcttgacatcctttgaccactccagaaaatagagcttccccttcgggggcacagtgaaagtggtgcatggttgtcgtcacctcgtgtcgggaaaagttgggttaagtccccca

SEQ ID NO: 73 - DNA, 992 bp, Enterococcus sp.
  misc_feature 19: note = n is a, c, g, or t
  misc_feature 422: note = n is a, c, g, or t
  misc_feature 425: note = n is a, c, g, or t
  misc_feature 943..945: note = n is a, c, g, or t
  misc_feature 971: note = n is a, c, g, or t
  misc_feature 987: note = n is a, c, g, or t
  source 1..992: mol_type = genomic DNA; note = isolate mc026cna1_d06
tcgaacgctttttctttcnccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtanaangttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccnnncttgacatcctttgaccactctaganatagagcttccccttnggggg

SEQ ID NO: 74 - DNA, 1075 bp, Enterococcus sp.
  source 1..1075: mol_type = genomic DNA; note = isolate mc071tn_fx_b1
ctatacatgcagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcacctcgtgtcctgagatgttgggttaagtcccgcaacaac

SEQ ID NO: 75 - DNA, 24 bp, synthetic construct
  source 1..24: mol_type = other DNA
cgaggtacaccgtaagttgttgct

SEQ ID NO: 76 - DNA, 24 bp, synthetic construct
  source 1..24: mol_type = other DNA
accaggacacgtagacttcacagt

SEQ ID NO: 77 - DNA, 23 bp, synthetic construct
  source 1..23: mol_type = other DNA
tggcccaaagatacgtgttccac

SEQ ID NO: 78 - DNA, 24 bp, synthetic construct
  source 1..24: mol_type = other DNA
cgaacaaaatcaggagctcgtcgt

SEQ ID NO: 79 - DNA, 20 bp, synthetic construct
  source 1..20: mol_type = other DNA
actgctgagtgcccacgctt

SEQ ID NO: 80 - DNA, 22 bp, synthetic construct
  source 1..22: mol_type = other DNA
gaggcacttgctagcgtcacac

SEQ ID NO: 81 - DNA, 22 bp, synthetic construct
  source 1..22: mol_type = other DNA
tgcgtgtaggcatcatccgtga

SEQ ID NO: 82 - DNA, 21 bp, synthetic construct
  source 1..21: mol_type = other DNA
gacacagcggcatcagcaagt

SEQ ID NO: 83 - DNA, 21 bp, synthetic construct
  source 1..21: mol_type = other DNA
cctttaccatggccgtgtagc

SEQ ID NO: 84 - DNA, 24 bp, synthetic construct
  source 1..24: mol_type = other DNA
ccatgaatggtgttcctccttctc

SEQ ID NO: 85 - DNA, 20 bp, synthetic construct
  source 1..20: mol_type = other DNA
aaacttgctgatgccgctgt

SEQ ID NO: 86 - DNA, 23 bp, synthetic construct
  source 1..23: mol_type = other DNA
ccgcctttaccgataaccatacc

SEQ ID NO: 87 - DNA, 20 bp, synthetic construct
  source 1..20: mol_type = other DNA
agagtttgatymtggctcag

SEQ ID NO: 88 - DNA, 21 bp, synthetic construct
  source 1..21: mol_type = other DNA
acggytaccttgttacgactt

SEQ ID NO: 89 - DNA, 1833 bp, Enterococcus avium
  misc_feature 11..12: note = n is a, c, g, or t
  source 1..1833: mol_type = genomic DNA; note = ATCC 14025 2
agagtttgatnntggctcaggacaaacgctggcggcgtgcctaatacatgcaagtcgaacgctttttctttcaccgagcttgctccaccgaaagaaaaggagtggcgaacgggtgagtaacacgtgggtaacctgcccatcagaaggggataacacttggaaacaggtgctaataccgtataacaataaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatccttgacggtatctaaccagaaagcacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcacactagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcggaaggtgcggctggatcacctcctttctaaggaaatagttcatcgaagaagttacggaaactacacaggaatgctttattcgttcagttttgagaggtctactctcaaatcgatccatggatagcgggctagcaaacattttaaagcaccgtagacacgcagtagatgagtttttgatagtcagtgcttttttgtgtatgaatggtgccgaatggtaagacctgcgagcactagctcgtgtatggcccgggagctgcctgggcgatgagacggatacccgtagctcggacctagtactgtcgatcaatgcgc

SEQ ID NO: 90 - DNA, 1546 bp, Enterococcus avium
  source 1..1546: mol_type = genomic DNA; note = 1
tgagtttgatcctggctcaggacgaacgctggcggcgtgcctaatacatgcaagtcgaacgctttttctttcaccggagcttgctccaycgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgargctcgaaagcgtggggagcaaacaggattagataccctggtagtccacgccgtaaacgatgartgctaagtgttggarggtttccgcccttcagtgctgcagctaacgcattaagcactccgcctggggartacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcggaaggtgcggctggatcacc

SEQ ID NO: 91 - DNA, 1524 bp, Enterococcus avium
  source 1..1524: mol_type = genomic DNA; note = ATCC 14025 1
ccgtagagtttgatcctggctcaggacgaacgctggcggcgtgcctaatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccctgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtc

SEQ ID NO: 92 - DNA, 1523 bp, Enterococcus avium
  source 1..1523: mol_type = genomic DNA; note = 2
cgctggcggcgtgcctaatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcggaaggtgcggctggatcacctcc

SEQ ID NO: 93 - DNA, 1520 bp, unidentified
  source 1..1520: mol_type = genomic DNA; note = Prior Sequence 1
cgttggtttaagcactctggctagagacgctggcggcgtgcctaatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactccagcgagactgccggtgacaaaccggaggaaggtggggatgacgtcaaatcatcatgccccttatgacctgggccacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggcgaggtaaccttttggagccagccgcctaaggtgggatagatgatgggggaagtcgagaaagtcgttgaa

SEQ ID NO: 94 - DNA, 1513 bp, unidentified
  source 1..1513: mol_type = genomic DNA; note = Prior Sequence 2
cttggagtctgctaggagacgctggcggcgtgcctaatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgatgggggaagtctatcaataccgaatgc

SEQ ID NO: 95 - DNA, 1507 bp, unidentified
  source 1..1507: mol_type = genomic DNA; note = Prior Sequence 3
atcagtactgctagagacgctggcggcgtgcctaatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatagaagccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgtcagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggccctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgatgggggaagtcataagaccgatc

SEQ ID NO: 96 - DNA, 1500 bp, Enterococcus malodoratus
  source 1..1500: mol_type = genomic DNA
ggacgaacgctggcggcgtgcctaatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacagtagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaacgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagc

SEQ ID NO: 97 - DNA, 1496 bp, Enterococcus raffinosus
  source 1..1496: mol_type = genomic DNA
gacgaacgctggcggcgtgcctaatacatgcaagtcgaacgccttttctttcaccggagcttgctccaccgaaagaaaaggagtggcgaacgggtgagtaacacgtgggtaacctgcccatcagaaggggataacacttggaaacaggtgctaataccgtataacaatagaaaccgcatggtttctatttgaaaggcgcttttgcgtcactgrtggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggt

SEQ ID NO: 98 - DNA, 1481 bp, unidentified
  source 1..1481: mol_type = genomic DNA; note = Prior Sequence 4
gacgaacgctggcggcgtgcctaatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacagtcgaaaccgcatggtttcgatttgaaaggcgcttttgcgtcactgatggatggacccgcggtgcattagctagttggtgaggtgacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgggtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtg

SEQ ID NO: 99 - DNA, 1461 bp, Enterococcus malodoratus
  source 1..1461: mol_type = genomic DNA; note = 2
acgaacgctggcggcgtgcctaatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacagtagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaacgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggt

SEQ ID NO: 100 - DNA, 1453 bp, Enterococcus avium
  source 1..1453: mol_type = genomic DNA; note = 3
catgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactggtggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattgggg

SEQ ID NO: 101 - DNA, 1429 bp, Enterococcus avium
  source 1..1429: mol_type = genomic DNA; note = ATCC 14025 3
acgctggcggcgtgcctaatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtgactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgagg

SEQ ID NO: 102 - DNA, 1425 bp, Enterococcus raffinosus
  source 1..1425: mol_type = genomic DNA; note = 2
gctggcggcgtgcctaatacatgcaagtcgaacgccttttctttcaccggagcttgctccaccgaaagaaaaggagtggcgaacgggtgagtaacacgtgggtaacctgcccatcagaaggggataacacttggaaacaggtgctaataccgtataacaatagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtga

SEQ ID NO: 103 - DNA, 1421 bp, Enterococcus gilvus
  source 1..1421: mol_type = genomic DNA
gagcttgctccaccgaaagaaaaggagtggcgaacgggtgagtaacacgtgggtaacctgcccatcagaaggggataacacttggaaacaggtgctaataccgtataacaatagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattgg

SEQ ID NO: 104 - DNA, 1472 bp, unidentified
  source 1..1472: mol_type = genomic DNA; note = Prior Sequence 5
acgctggcggcgtgcctatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtggatagagactgggg

SEQ ID NO: 105 - DNA, 1469 bp, unidentified
  source 1..1469: mol_type = genomic DNA; note = Prior Sequence 6
cgcgagctgcggcgtcctatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtggaagatatgg

SEQ ID NO: 106 - DNA, 1507 bp, unidentified
  source 1..1507: mol_type = genomic DNA; note = Prior Sequence 7
cgaacgctggcggcgtgcctaatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcgga

SEQ ID NO: 107 - DNA, 1337 bp, unidentified
  source 1..1337: mol_type = genomic DNA; note = Prior Sequence 8
gctttttctttcaccggagcttgctccaccgaaagaaaaagagtggcgaacgggtgagtaacacgtgggtaacctgcccatcagaaggggataacacttggaaacaggtgctaataccgtataacaagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccggg

SEQ ID NO: 108 - DNA, 1344 bp, unidentified
  source 1..1344: mol_type = genomic DNA; note = Prior Sequence 9
gtcgaacgctttttctttcaccggagcttgctccaccgaaagaaaaagagtggcgaacgggtgagtaacacgtgggtaacctgcccatcagaaggggataacacttggaaacaggtgctaataccgtataacaagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaaagttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccggg

SEQ ID NO: 109 - DNA, 1309 bp, unidentified
  source 1..1309: mol_type = genomic DNA; note = Prior Sequence 10
tgcaagtcgtacgctggctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgct

SEQ ID NO: 110 - DNA, 1366 bp, unidentified
  source 1..1366: mol_type = genomic DNA; note = Prior Sequence 11
ctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagag

SEQ ID NO: 111 - DNA, 1116 bp, unidentified
  source 1..1116: mol_type = genomic DNA; note = Prior Sequence 12
gcggcgtgcctaatacatgcaagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggagcgagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatt

SEQ ID NO: 112 - DNA, 1323 bp, unidentified
  source 1..1323: mol_type = genomic DNA; note = Prior Sequence 13
agtggcgaacgggtgagtaacacgtgggtaacctgcccatcagaaggggataacacttggaaacaggtgctaataccgtataacaatcgaaaccgcatggtttcgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacacca

SEQ ID NO: 113 - DNA, 1417 bp, unidentified
  source 1..1417: mol_type = genomic DNA; note = Prior Sequence 14
agagtttgatcctggctcaggacgaacgctggcggcgtgcctaatacatgcaagtcgtacgctttttctttcatcggagcttgctccaccgaaagaaaaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcgactctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgt

SEQ ID NO: 114 - RNA, 1535 nt, unidentified
  source 1..1535: mol_type = genomic RNA; note = Prior sequence 15
cctggctcaggacgaacgctggcggcgtgcctaatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcggaaggtgcggctggatca

SEQ ID NO: 115 - DNA, 1401 bp, Enterococcus sp.
  misc_feature 333: note = n is a, c, g, or t
  source 1..1401: mol_type = genomic DNA; note = Pep7
acgcctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgnaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaac

SEQ ID NO: 116 - DNA, 1295 bp, Enterococcus gilvus
  source 1..1295: mol_type = genomic DNA; note = 2
acgctttttctttcaccggagcttgctccaccgaaagaaaaggagtggcgaacgggtgagtaacacgtgggtaacctgcccatcagaaggggataacacttggaaacaggtgctaataccgtataacaatagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctgaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcg

SEQ ID NO: 117 - DNA, 1275 bp, Enterococcus raffinosus
  source 1..1275: mol_type = genomic DNA; note = 3
gggtgagtaacacgtgggtaacctgcccatcagaaggggataacacttggaaacaggtgctaataccgtataacaatagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaca

SEQ ID NO: 118 - DNA, 1183 bp, Enterococcus sp.
  source 1..1183: mol_type = genomic DNA; note = Da-20
gtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcaatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggcagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctac

SEQ ID NO: 119 - DNA, 1133 bp, Enterococcus sp.
  source 1..1133: mol_type = genomic DNA; note = 936B6_12ASB
gtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgsaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccgga

SEQ ID NO: 120 - DNA, 1422 bp, unidentified
  source 1..1422: mol_type = genomic DNA; note = Prior Sequence 16
acgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcgatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaag

SEQ ID NO: 121 - DNA, 1431 bp, unidentified
  source 1..1431: mol_type = genomic DNA; note = Prior Sequence 17
tgcagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaag

SEQ ID NO: 122 - DNA, 1410 bp, unidentified
  source 1..1410: mol_type = genomic DNA; note = Prior Sequence 18
agtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttt

SEQ ID NO: 123 - DNA, 1295 bp, unidentified; identical to SEQ ID NO: 116
  source 1..1295: mol_type = genomic DNA; note = Prior Sequence 19

SEQ ID NO: 124 - DNA, 1524 bp, unidentified; identical to SEQ ID NO: 91
  source 1..1524: mol_type = genomic DNA; note = Prior Sequence 20

SEQ ID NO: 125 - DNA, 1521 bp, unidentified
  source 1..1521: mol_type = genomic DNA; note = Prior Sequence 21
ctcaggacgaacgctggcggcgtgcctaatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggtttcggtttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgcaagtctgaccgagcaacgccgcgtgagtgaagaaggttttcggatcgtaaaactctgttgttagagaagaacaaggatgagagtagaaygttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagrtaatctyttaaagcttctctcagttcggattgtaggctgraactcgcctacatgaagccggaatcgctagtaatcgcggatcagcacgccgcggtgaatacgttcccgggccttgtacacaccgcrcgtcacaccacgagagtttgtaacacccgaagtcggtgaggtaaccttttggagccagccgcctaaggtgggatagatgattggggtgaagtcgtaacaaggtagccgtatcggaaggtgcgg

SEQ ID NO: 126 - DNA, 1893 bp, Enterococcus raffinosus
  misc_feature 11..12: note = n is a, c, g, or t
  source 1..1893: mol_type = genomic DNA; note = 4
agagtttgatnntggctcaggacgaacgctggcggcgtgcctaatacatgcaagtcgaacgccttttctttcaccggagcttgctccaccgaaagaaaaggagtggcgaacgcgtgagtaacacgtgggtaacctgcccatcagaaggggataacacttggaaacaggtgctaataccgtataacaatagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtagaatgttcatcccttgacggtatctaaccagaaagccacggctaactaacgtgccagcagccgcggtaatacgtagagtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttcggcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcgaagggtgcggctggatcacctcctttctaaggaatattacggaaactacacaggttgctttattcgttcagttttgagaggtctactctcaaatcgttcattgaacactggatacagaaacgataatttctacaaaccgagaacacgcgttgaatgagttttttaataagtcattgctattttttctgattgtgagtctatcgctagtggacactcacccctaccgtagtagataagttagtgaataggcgccgtgatgcgtgtcttgcgcgtgaggcggcctacacgatatgctcggcactgtatgctgcgatcgtgctgcgcacacgatatgcttcggggagctgtaagtaattctttcgggg

SEQ ID NO: 127 - DNA, 1701 bp, Enterococcus malodoratus
  misc_feature 11..12: note = n is a, c, g, or t
  source 1..1701: mol_type = genomic DNA; note = 3
agagtttgatnntggctcgacgaacgctggcggcgtgcctaatacatgcaagtcgaacgctttttctttcaccgagcttgctccaccgaaagaaaaggagtcgcgaacggctgagtaacacgtgggtaacctgcccatcagaaggagataacacttgaaacaggtgctaataccgtataacagtagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagataaaacgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagacaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcgtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgtgatagatgattggggtgaagtcgtaacaaggtagccgtatcggaaggtgcggctggatcacctcctttctaaggaatattacggagactacacaggttgctttattcgttcagttttgagaggtttactctcaaaaatcgttcattgaaactggtacgacaaccaaacaaaccggaacaccgcgttgatgagttttttaatagttcattgctattttttt

SEQ ID NO: 128 - DNA, 1535 bp, Enterococcus hirae
  source 1..1535: mol_type = genomic DNA
cctggctcaggacgaacgctggcggcgtgcctaatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcggaaggtgcggctggatca

SEQ ID NO: 129 - DNA, 1535 bp, Enterococcus hirae; identical to SEQ ID NO: 128
  source 1..1535: mol_type = genomic DNA; note = 2

SEQ ID NO: 130 - DNA, 1530 bp, unidentified
  source 1..1530: mol_type = genomic DNA; note = Prior Sequence 22
cttagagtttgatcctggctcaggacgaacgctggcggcgtgcctaatacatgcaagtcgaacgctttttttttcaccggagcttgctccaccgaaagaaaaggagtggcgaacgggtgagtaacacgtgggtaacctgcccatcagaaggggataacacttggaaacaggtgctaataccgtataacaatagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaagcttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccccggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttgagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtaaccgtaaagg

SEQ ID NO: 131 - DNA, 1513 bp, Enterococcus pseudoavium
  source 1..1513: mol_type = genomic DNA
cgcacgaacgctggcggcgtgcctaatacatgcaagtcgaacgctttttctttcaccggagcttgctccaccgaaagaaaaagagtggcgaacgggtgagtaacacgtgggtaacctgcccatcagaaggggataacacttggaaacaggtgctaataccgtataacaagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcggaagg

SEQ ID NO: 132 - DNA, 1507 bp, Enterococcus hirae; identical to SEQ ID NO: 106
  source 1..1507: mol_type = genomic DNA; note = 3

SEQ ID NO: 133 - DNA, 1500 bp, Enterococcus viikkiensis
  source 1..1500: mol_type = genomic DNA
gcggcgtgcctaatacatgcaagtcgaacgctttttctttcaccggagcttgctccaccgaaagaaaaagagtggcgaacgggtgagtaacacgtgggtaacctgcccatcagaaggggataacacttggaaacaggtgctaataccgtataacaagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcggaagg

SEQ ID NO: 134 - DNA, 1500 bp, Enterococcus viikkiensis; identical to SEQ ID NO: 133
  source 1..1500: mol_type = genomic DNA; note = 2

SEQ ID NO: 135 - DNA, 1497 bp, Enterococcus hirae
  source 1..1497: mol_type = genomic DNA; note = 4
ggacgaacgctggcggcgtgcctaatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaagg

SEQ ID NO: 136 - DNA, 1497 bp, Enterococcus viikkiensis
  source 1..1497: mol_type = genomic DNA; note = 3
gcggcgtgcctaatacatgcaagtcgaacgctttttctttcaccggagcttgctccaccgaaagaaaagagtggcgaacgggtgagtaacacgtgggtaacctgcccatcagaaggggataacacttggaaacaggtgctaataccgtataacaagtaaaaccgcatggttttactttgaaaggcgcctttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcgtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaagttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcggaagg

SEQ ID NO: 137 - DNA, 1494 bp, Enterococcus devriesei
  source 1..1494: mol_type = genomic DNA
tttgatcctggctgaggacgaacgctggcggcgtgcctaatacatgcaagtcgaacgctttttctttcaccggagcttgctccaccgaaagaaaaagagtggcgaacgggtgagtaacacgtgggtaacctgcccatcagaaggggataacacttggaaacaggtgctaataccgtataacaagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaaagttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggg

SEQ ID NO: 138 - DNA, 1493 bp, Enterococcus devriesei
  source 1..1493: mol_type = genomic DNA; note = 2
tttgatcctggctgaggacgaacgctggcggcgtgcctaatacatgcaagtcgaacgctttttctttcaccggagcttgctccaccgaaagaaaaagagtggcgaacgggtgagtaacacgtgggtaacctgcccatcagaaggggataacacttggaaacaggtgctaataccgtataacaagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaaagttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattgg

SEQ ID NO: 139 - DNA, 1490 bp, Enterococcus devriesei
  source 1..1490: mol_type = genomic DNA; note = 3
gatcctggctgaggacgaacgctggcggcgtgcctaatacatgcaagtcgaacgctttttctttcaccggagcttgctccaccgaaagaaaaagagtggcgaacgggtgagtaacacgtgggtaacctgcccatcagaaggggataacacttggaaacaggtgctaataccgtataacaagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcgktttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattgg

SEQ ID NO: 140 - DNA, 1485 bp, Enterococcus thailandicus
  source 1..1485: mol_type = genomic DNA
tatacatgcaagtcgtacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcgactctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgaaagtttgtaacacccgaagtcggtgaggtaacctttttggagccagccgccctaaggtgggataaatgattggggtgaagtcctaacaaggtaaccgtaa

SEQ ID NO: 141 - DNA, 1483 bp, unidentified
  source 1..1483: mol_type = genomic DNA; note = Prior Sequence 23
gacgaacgctggcggcgtgcctaatacatgcaagtcgtacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagaggagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcgactctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaacccaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgaaagtttgtaacacccgaagtcggtgaggtaacctttttggagccagccgcctaaggtgggatagatgattggggtg

SEQ ID NO: 142 - DNA, 1483 bp, unidentified
  source 1..1483: mol_type = genomic DNA; note = Prior Sequence 24
gacgaacgctggcggcgtgcctaatacatgcaagtcgtacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcgactctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagtccggattgtaggctgcaactcgcctacgtgaagccggaatcgctagtaatcgcggatcagcacgccgcggtgaatacgttcccgggccttgtacacaccgcccgtcacaccacgaaagtttgtaacacccgaagtcggtgaggtaacctttttggagccagccgcctaaggtgggatagatgattggggtg

SEQ ID NO: 143 - DNA, 1483 bp, Enterococcus thailandicus
  source 1..1483: mol_type = genomic DNA; note = 2
gacgaacgctggcggcgtgcctaatacatgcaagtcgtacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcgactctctggtctgyaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgaaagtttgtaacacccgaagtcggtgaggtaacctttttggagccagccgcctaaggtgggatagatgattggggtg

SEQ ID NO: 144 - DNA, 1482 bp, Enterococcus sp.
  source 1..1482: mol_type = genomic DNA; note = Enterococcus hawaiiensis
gacgaacgctggcggcgtgcctaatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataataagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaacgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgattggggtg

SEQ ID NO: 145 - DNA, 1481 bp, Enterococcus sp.
  source 1..1481: mol_type = genomic DNA; note = 4
caccccaatcatctatcccaccttaggcggctggctccaaaggttacctcaccgacttcgggtgttacaaactttcgtggtgtgacgggcggtgtgtacaaggcccgggaacgtattcaccgcggcgtgctgatccgcgattactagcgattccggcttcatgtaggcgagttgcagcctacaatccgaactgagagaagctttaagagattagcttagcctcgcgacttcgcgactcgttgtacttcccattgtagcacgtgtgtagcccaggtcataaggggcatgatgatttgacgtcatccccaccttcctccggtttgtcaccggcagtcttgctagagtgcccaacttaatgatggcaactaacaataagggttgcgctcgttgcgggacttaacccaacatctcacgacacgagctgacgacaaccatgcaccacctgtcactttgcccccgaaggggaagctctatctctagagtggtcaaaggatgtcaagacctggtaaggttcttcgcgttgcttcgaattaaaccacatgctccaccgcttgtgcgggcccccgtcaattcctttgagtttcaaccttgcggtcgtactccccaggcggagtgcttaatgcgttagctgcagcactgaagggcggaaaccctccaacacttagcactcatcgtttacggcgtggactaccagggtatctaatcctgtttgctccccacgctttcgagcctcagcgtcagttacagaccagagagtcgccttcgccactggtgttcctccatatatctacgcatttcaccgctacacatggaattccactctcctcttctgcactcaagtctcccagtttccaatgaccctccccggttgagccgggggctttcacatcagacttaagaaaccgcctgcgctcgctttacgcccaataaatccggacaacgcttgccacctacgtattaccgcggctgctggcacgtagttagccgtggctttctggttagataccgtcaagggatgaacagttactctcatccttgttcttctctaacaacagagttttacgatccgaaaaccttcttcactcacgcggcgttgctcggtcagactttcgtccattgccgaagattccctactgctgcctcccgtaggagtttgggccgtgtctcagtcccaatgtggccgatcaccctctcaggtcggctatgcatcgtggccttggtgagccgttacctcaccaactagctaatgcaccgcgggtccatccatcagcgacacccgaaagcgcctttcaaatcaaaaccatgcggtttcgattgttatacggtattagcacctgtttccaagtgttatccccttctgatgggcaggttacccacgtgttactcacccgttcgccactccttttctttcggtggagcaagctccggagaaagaaaaagcgtacgacttgcatgtattaggcacgccgccagcgttcgtc

SEQ ID NO: 146 - DNA, 1477 bp, Enterococcus hirae
  source 1..1477: mol_type = genomic DNA; note = 5
atgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgta

SEQ ID NO: 147 - DNA, 1473 bp, Enterococcus hirae
  source 1..1473: mol_type = genomic DNA; note = 6
gagtttgatcctggctcagaacgaacgctggcggcgtgcttaacacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaacctttggagccagccg

SEQ ID NO: 148 - DNA, 1473 bp, Enterococcus viikkiensis
  source 1..1473: mol_type = genomic DNA; note = 4
gcggcgtgcctaatacatgcaagtcgaacgctttttctttcaccggagcttgctccaccgaaagaaaaagagtggcgaacgggtgagtaacacgtgggtaacctgcccatcagaaggggataacacttggaaacaggtgctaataccgtataacaagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaag

SEQ ID NO: 149 - DNA, 1464 bp, Enterococcus raffinosus
  source 1..1464: mol_type = genomic DNA; note = 5
agcgagtgcgcttgctatacatgcagtcgaacgctttttctttcaccggagcttgctccaccgaaagaagaggagtggcgaacgggtgagtaacacgtgggtaacctgcccatcagaaggggataacacttggaaacaggtgctaataccgtataacagtagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagaataaaacgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtgggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgatagtattaa

SEQ ID NO: 150 - DNA, 1462 bp, Enterococcus gilvus
  source 1..1462: mol_type = genomic DNA; note = 3
gcggcgtgcctaatacatgcaagtcgacgctttttctttcaccggagcttgctccaccgaaagaaaaggagtggcgaacgggtgagtaacacgtgggtaacctgcccatcagaaggggataacacttggaaacaggtgctaataccgtataacaatagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtkaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagtaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgatt

SEQ ID NO: 151 - DNA, 1460 bp, Enterococcus pseudoavium
  misc_feature 152: note = n is a, c, g, or t
  misc_feature 171: note = n is a, c, g, or t
  source 1..1460: mol_type = genomic DNA; note = 2
ggctcaggacgaagctggcggcgtgcctaatacatgcaagtcgaacgctttttctttcaccggagcttgctccaccgtaaagaaaaagagtggcgaacgggtgtagtaacacgtgggtaacctgcccatcagaaggggataacacttgcgknracaggtgctaataccagntataacaagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagc

SEQ ID NO: 152 - DNA, 1455 bp, Enterococcus gilvus
  source 1..1455: mol_type = genomic DNA; note = 4
tacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaatagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcgtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctaggagataggagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggg

SEQ ID NO: 153 - DNA, 1449 bp, Enterococcus durans
  source 1..1449: mol_type = genomic DNA
gtygtacgcttctttttccatcggagcttgctcaycgggaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttcttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagc

SEQ ID NO: 154 - DNA, 1449 bp, Enterococcus malodoratus
  misc_feature 1244: note = n is a, c, g, or t
  misc_feature 1254: note = n is a, c, g, or t
  misc_feature 1412: note = n is a, c, g, or t
  misc_feature 1426: note = n is a, c, g, or t
  misc_feature 1434: note = n is a, c, g, or t
  misc_feature 1440: note = n is a, c, g, or t
  source 1..1449: mol_type = genomic DNA; note = 4
gggcagcatatatgcagtcaacgctttttctttcaccggagcttgctccaccgaaagaaaaagagtggcgaacgggtgagtaacacgtgggtaacctgcccatcagaaggggataacacttggaaacaggtgctaataccgtataacagtagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaacgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggntaagctaatntcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcgtngaggtaacctttgnagccagcngcttangtgtttatt

SEQ ID NO: 155 - DNA, 1447 bp, Enterococcus faecium
  source 1..1447: mol_type = genomic DNA
ttgggcgtttcgatgccagttatcgttccacacggaaaaagaggccctcgtgggaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccgggatttattgggcgtaaagcgagcgcaggcggtttcttaagtctgatgtgaaagcccccggctcaacctggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtgggtttaattcgaagcaacgcgaagaaccttaccaggtcttgacatcctttgaccacctctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggttgaggtaacctttggagccagcccggccttaaaagggggatatagt

SEQ ID NO: 156 - DNA, 1441 bp, Enterococcus raffinosus
  misc_feature 17: note = n is a, c, g, or t
  misc_feature 28: note = n is a, c, g, or t
  misc_feature 1409: note = n is a, c, g, or t
  misc_feature 1418: note = n is a, c, g, or t
  misc_feature 1438: note = n is a, c, g, or t
  source 1..1441: mol_type = genomic DNA; note = 6
ggctatcatgcagtcgnacgctttttcnatcaccggagcttgctccaccgaaagaaaaggagtggcgaacgggtgagtaacacgtgggtaacctgcccatcagaaggggataacacttggaaacaggtgctaataccgtataacaatagaaaccgcatggtttctatttgaaaggcgcttttgcgccactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcgtngaggtaacnctttggagccagccgcctanggg

SEQ ID NO: 157 - DNA, 1437 bp, Enterococcus sp.
  source 1..1437: mol_type = genomic DNA; note = 115w
gctataatgcagtcgtacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcgactctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcattctagcaagactgccggtgacaaaccggaggaaggtggggaggaggtcaaatcatcatgccccttatgacctgggctacacacgtgttccaatgggaagtacaaggagtcgggaagtcgggaggttaagttaatttcttaaagcttttttcagttcggattgtaggctgcaattcgcctacatgaagccggaatcgttagtaatcgcggatcaccacgccgcggggaatacgttcccggcccttgtacacaccgcccgtcacaccacgaaagtttgtaacaccggaagtcggtgaggtaaccttttggagccagccgccta

SEQ ID NO: 158 - DNA, 1435 bp, Enterococcus durans
  source 1..1435: mol_type = genomic DNA; note = 2
cgcttctttttcatcggagcttgctcaycgggaaaagaagagtggcgaacgggtgagtaacacgtgggtaactgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttcccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattgg

SEQ ID NO: 159 - DNA, 1435 bp, Enterococcus sp.
  source 1..1435: mol_type = genomic DNA; note = Enterococcus xiangfangensis
cgctttttctttcaccggagcttgctccaccgaaagaaaaagagtggcgaacgggtgagtaacacgtgggtaacctgcccatcagaaggggataacacttggaaacaggtgctaataccgtataacaagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaaagttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatga

SEQ ID NO: 160 - DNA, 1431 bp, unidentified
  source 1..1431: mol_type = genomic DNA; note = bacterium MRG-IFC-2
aaatatgcagtcgtacgcttctttttaccggagcttgctccaccggaaaaagggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcat

SEQ ID NO: 161 - DNA, 1427 bp, Enterococcus sp.
  source 1..1427: mol_type = genomic DNA; note = KS40
tgcagtcgacgctttttctttcaccggagcttgctccaccgaaagaaaaggagtggcgaacgggtgagtaacacgtgggtaacctgcccatcagaaggggataacacttggaaacaggtgctaataccgtataataagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaacgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcct

SEQ ID NO: 162 - DNA, 1426 bp, Enterococcus faecalis
  source 1..1426: mol_type = genomic DNA
agtcgtacgcttctttttcaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcct

SEQ ID NO: 163 - DNA, 1425 bp, Enterococcus viikkiensis
  source 1..1425: mol_type = genomic DNA; note = 5
accggagcttgctcaccgaaagaaaaagagtggcgaacgggtgagtaacacgtgggtaacctgcccatcagaaggggataacacttggaaacaggtgctaataccgtataacaagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttcttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtg

SEQ ID NO: 164 - DNA, 1424 bp, Enterococcus pseudoavium
  source 1..1424: mol_type = genomic DNA; note = 3
tggcggcgtgcctaatacatgcaagtcgaacgctttttctttcaccggagcttgctccaccgaaagaaaaagagtggcgaacgggtgagtaacacgtgggtaacctgcccatcagaaggggataacacttggaaacaggtgctaataccgtataacaagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgag

SEQ ID NO: 165 - DNA, 1419 bp, Enterococcus sp.
  source 1..1419: mol_type = genomic DNA; note = SB
tgcagtcgacgcctctttttccaccggagcttgctccatcggaaaaagaggagtggcgaacgggtgagtaacacgtgggtaacctgcccatcagaaggggataacacttggaaacaggtgctaataccgtataacaatcgaaaccgcatggtttcgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgaacacccgaagtcggtgaggtaacctttggagcca

SEQ ID NO: 166 - DNA, 1416 bp, unidentified
  source 1..1416: mol_type = genomic DNA; note = bacterium mpn-isolate group 13
tctatcccaccttaggcggctggctccaaaaggttacctcaccgacttcgggtgttacaaactctcgtggtgtgacgggcggtgtgtacaaggcccgggaacgtattcaccgcggcgtgctgatccgcgattactagcgattccggcttcatgtaggcgagttgcagcctacaatccgaactgagagaagctttaagagattagcttagcctcgcgactttgcgactcgttgtacttcccattgtagcacgtgtgtagcccaggtcataaggggcatgatgatttgacgtcatccccaccttcctccggtttgtcaccggcagtcttgctagagtgcccaactaaatgatggcaactaacaataagggttgcgctcgttgcgggacttaacccaacatctcacgacacgagctgacgacaaccatgcaccacctgtcactttgcccccgaaggggaagctctatctctagagtggtcaaaggatgtcaagacctggtaaggttcttcgcgttgcttcgaattaaaccacatgctccaccgcttgtgcgggcccccgtcaattcctttgagtttcaaccttgcggtcgtactccccaggcggagtgcttaatgcgttagctgcagcactgaagggcggaaaccctccaacacttagcactcatcgtttacggcgtggactaccagggtatctaatcctgtttgctccccacgctttcgagcctcagcgtcagttacagaccagagagccgccttcgccactggtgttcctccatatatctacgcatttcaccgctacacatggaattccactctcctcttctgcactcaagtctcccagtttccaatgaccctccccggttgagccgggggctttcacatcagacttaagaaaccgcctgcgctcgctttacgcccaataaatccggacaacgcttgccacctacgtattaccgcggctgctggcacgtagttagccgtggctttctggttagataccgtcaagggatgaacagttactctcatccttgttcttctctaacaacagagttttacgatccgaaaaccttcttcactcacgcggcgttgctcggtcagactttcgtccattgccgaagattccctactgctgcctcccgtaggagtttgggccgtgtctcagtcccaatgtggccgatcaccctctcaggtcggctatgcatcgtcgccttggtgagccgttacctcaccaactagctaatgcaccgcgggtccatccatcagcgacacccgaaagcgcctttcaaatcaaaaccatgcggtttcgattgttatacggtattagcacctgtttccaagtgttatccccttctgatgggcaggttacccacgtgttactcacccgttcgccactcctctttttccggtggagcaagctct

SEQ ID NO: 167 - DNA, 1408 bp, Enterococcus avium
  source 1..1408: mol_type = genomic DNA; note = 4
ctgcccatcagaaggggataacacttggaaacaggtgctaataccgtataacagtagaaaccgcatggtttctatttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaaaacgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtgggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatggggagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggtagccgtatcggaaggtgcggctggat

SEQ ID NO: 168 - DNA, 1401 bp, Enterococcus faecium
  source 1..1401: mol_type = genomic DNA; note = 2
gcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacct

SEQ ID NO: 169 - DNA, 1397 bp, Enterococcus faecium
  source 1..1397: mol_type = genomic DNA; note = 3
gcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggta

SEQ ID NO: 170 - DNA, 1397 bp, Enterococcus faecium
  source 1..1397: mol_type = genomic DNA; note = 4
ttctttttcaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacc

SEQ ID NO: 171 - DNA, 1396 bp, unidentified
  misc_feature 824: note = n is a, c, g, or t
  source 1..1396: mol_type = genomic DNA; note = Prior Sequence 25
gacgaacgctggcggcgtgcctaatacatgcaagtcgaacgctttttctttcaccggagcttgctccaccgaaagaaaaggagtggcgaacgggtgagtaacacgtgggtaacctgcccatcagaaggggataacacttggaaacaggtgctaataccgtataacaagtaaaaccgcatggttttactttgaaaggcgcttttgcgtcactgatggatggacccgcggtgcattagctagttggtgaggtaacggctcaccaaggcaacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtagaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggaaacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgtnggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagtttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacattgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgt

SEQ ID NO: 172 - DNA, 1392 bp, Enterococcus faecium
  source 1..1392: mol_type = genomic DNA; note = 5
aacgggtgagtaacacgtgggtaacctgcccatcagaaggggataacacttggaaacaggtgctaataccgtataacaatcgaaaccgcatggttttgtttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttggagccagccgcctaaggtggatagatgatgggggaag

SEQ ID NO: 173 - DNA, 1383 bp, unidentified
  source 1..1383: mol_type = genomic DNA; note = bacterium MRG-2
ccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggt

SEQ ID NO: 174 - DNA, 1381 bp, unidentified
  source 1..1381: mol_type = genomic DNA; note = Prior Sequence 26
gacgaacgctggcggcgtgcctaatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggcct

SEQ ID NO: 175 - DNA, 1379 bp, Enterococcus faecium
  source 1..1379: mol_type = genomic DNA; note = 6
ctccaccggaaaaagaggagtggcgaacgggtgagtaacacgtgggtaacctgcccatcagaaggggataacacttggaaacaggtgctaataccgtataacaatcaaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctt

SEQ ID NO: 176 - DNA, 1379 bp, Enterococcus faecium; identical to SEQ ID NO: 175
  source 1..1379: mol_type = genomic DNA; note = 7

SEQ ID NO: 177 - DNA, 1379 bp, Enterococcus faecium; identical to SEQ ID NO: 175
  source 1..1379: mol_type = genomic DNA; note = 8

SEQ ID NO: 178 - DNA, 1379 bp, Enterococcus faecium; identical to SEQ ID NO: 175
  source 1..1379: mol_type = genomic DNA; note = 9

SEQ ID NO: 179 - DNA, 1366 bp, Enterococcus mundtii
  source 1..1366: mol_type = genomic DNA
agaggagtggcgaacgggtgagtaacacgtgggtaacctgcccatcagaaggggataacacttggaaacaggtgctaataccgtataacaatcgaaaccgcatggtttcgttttgaaaggcgctttacggtgccgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaagggtgagagtaactgttcaccccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtgggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcgggtgaaaaacgttcccggggccttgtacacacccgcccgtcacaccacggagagtttgtaacaccccgaagtcggtgaag

SEQ ID NO: 180 - DNA, 1362 bp, Enterococcus mundtii
  source 1..1362: mol_type = genomic DNA; note = 2
agaggagtggcgaacgggtgagtaacacgtgggtaacctgcccatcagaaggggataacacttggaaacaggtgctaataccgtataacaatcgaaaccgcatggtttcgttttgaaaggcgctttacggtgccgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaagggtgagagtaactgttcaccccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtgggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcacacgccgcggggaaaaacctttcccgggccttgtacacaccgcccgtcacaccaccgagagtttgtaacacccgaagtcggtgag

SEQ ID NO: 181 - DNA, 1361 bp, Enterococcus mundtii
  source 1..1361: mol_type = genomic DNA; note = 3
agaggagtggcgaacgggtgagtaacacgtgggtaacctgcccatcagaaggggataacacttggaaacaggtgctaataccgtataacaatcgaaaccgcatggtttcgttttgaaaggcgctttacggtgccgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaagggtgagagtaactgttcaccccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcgggtgaatacgtttcccgggccttgtacacaccgcccgtcacaccacgaagagtttgtaacacccgaagtcggtgag

SEQ ID NO: 182 - DNA, 1359 bp, Enterococcus faecium
  source 1..1359: mol_type = genomic DNA; note = 10
agaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttggagccagccgcctaaggtggatagatgatgggggaag

SEQ ID NO: 183 - DNA, 1340 bp, Enterococcus faecium
  source 1..1340: mol_type = genomic DNA; note = 11
ctttttccaccggagcttgctccaccggaaaaagaggagtggcgaacggggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttg

SEQ ID NO: 184 - DNA, 1339 bp, Enterococcus faecium
  source 1..1339: mol_type = genomic DNA; note = 12
aaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagt

SEQ ID NO: 185 - DNA, 1337 bp, Enterococcus sp.
  source 1..1337: mol_type = genomic DNA; note = LV 07
tcttttcccaccggagcttgctccagggggtaaaagaggagtggcgaacgggtgagtaacacgtgggtaacctgcccatcagaaggggataacacttggaaacaggtgctaataccgtataacaatcgaaaccgcatggtttcgttttgaaaggcgctttacggtgccgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaagggtgagagtaactgttcaccccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgccacatgaagccggaatcgctagtaatcgcggatcagcacgccgcggtgaatacgttcccgggcc

SEQ ID NO: 186 - DNA, 1335 bp, Enterococcus hirae
  source 1..1335: mol_type = genomic DNA; note = 7
gagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttg

SEQ ID NO: 187 - DNA, 1334 bp, Enterococcus faecium
  source 1..1334: mol_type = genomic DNA; note = LV 07
ctccaccggaaaaagaggagtggcgaacgggtgagtaacacgtgggtaacctgcccatcagaaggggataacacttggaaacaggtgctaataccgtataacaatcaaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgt

SEQ ID NO: 188 - DNA, 1334 bp, Enterococcus faecium
  source 1..1334: mol_type = genomic DNA; note = 14
ccaccggagcttgctccaccggaaaaagaggagtggcgaacgggtgagtaacacgtgggtaacctgcccatcagaaggggataacacttggaaacaggtgctaataccgtataacaatcaaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgt

SEQ ID NO: 189 - DNA, 1320 bp, Enterococcus mundtii
  source 1..1320: mol_type = genomic DNA; note = QU 25
tggcgaacgggtgagtaacacgtgggtaacctgcccatcagaaggggataacacttggaaacaggtgctaataccgtataacaatcgaaaccgcatggtttcgttttgaaaggcgctttacggtgccgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaagggtgagagtaactgttcaccccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcgaagtcgcgaggctaagctaatcttttaaagcttctctcagttcggattgtaggctgcaactcgcctacatgaagccggaatcgctagtaatcgcggatcagcacgccgcggtgaatacgttcccgggccttgtacacaccgcccgtcacacc

SEQ ID NO: 190 - DNA, 1265 bp, unidentified
  source 1..1265: mol_type = genomic DNA; note = Prior Sequence 27
ggaaaaagaggagtggcgaacgggtgagtaacacgtgggtaacctgcccatcagaaggggataacacttggaaacaggtgctaataccgtataacaatcaaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgc

SEQ ID NO: 191 - DNA, 1165 bp, Enterococcus faecium
  source 1..1165: mol_type = genomic DNA; note = 15
ggcgtgcctaatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcaaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtgggg

SEQ ID NO: 192 - DNA, 1148 bp, Enterococcus sp.
  source 1..1148: mol_type = genomic DNA; note = 618B2_12AGalle
acatgcaagtcgaacgcttctttttccaccggagcttgctccaccggaaaaagaggagtggcgaacgggtgagtaacacgtgggtaacctgcccatcagaaggggataacacttggaaacaggtgctaataccgyataacaatcaaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggagaaagg

SEQ ID NO: 193 - DNA, 1138 bp, Enterococcus sp.
  source 1..1138: mol_type = genomic DNA; note = 691B1_12EGalle
gtcgaacgcttctttttccaccggagcttgctccaccggaaaaagaggagtggcgaacgggtgagtaacacgtgggtaacctgcccatcagaaggggataacacttggaaacaggtgctaataccgtataacaatcraaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaa

SEQ ID NO: 194 - DNA, 1130 bp, Enterococcus sp.
  source 1..1130: mol_type = genomic DNA; note = 680B1_12ESB
gtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaac

SEQ ID NO: 195 - DNA, 1126 bp, Enterococcus sp.
  source 1..1126: mol_type = genomic DNA; note = A3
tatagagtttgatccctggctcaggacgaacgctggcggcgtgcctaatacatgcaagtcgtacgctttttctttcaccggagcttgctccaccgaaagaaaaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggkatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcgactctctggtctgyaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgca

SEQ ID NO: 196 - DNA, 1124 bp, Enterococcus sp.
  source 1..1124: mol_type = genomic DNA; note = 366B7_12EEMB
atgcaagtcgtacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactg

SEQ ID NO: 197 - DNA, 1116 bp, Enterococcus sp.
  source 1..1116: mol_type = genomic DNA; note = 545B2_12AVRE
acatgcaagtcgaacgctttttctttcaccggagcttgctccaccgaaagaaaaagagtggcgaacgggtgagtaacacgtgggtaacctgcccatcagaaggggataacacttggaaacaggtgctaataccgtataacactattttccgcatggaagaaagttgaaaggcgcttttgcgtcactgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggatgagagtagaacgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcgaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactcta

SEQ ID NO: 198 - DNA, 1113 bp, Enterococcus sp.
  source 1..1113: mol_type = genomic DNA; note = 692B1_12AGalle
cggagcttgctccaccggagaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccgga

SEQ ID NO: 199 - DNA, 1112 bp, Enterococcus sp.
  source 1..1112: mol_type = genomic DNA; note = 325B3_12AGalle
acatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggca

SEQ ID NO: 200 - DNA, 1110 bp, Enterococcus sp.
  source 1..1110: mol_type = genomic DNA; note = 759B5_12ASB
tgcaagtcgaacgctttttctttcaccggagcttgctccaccgaaagaaaaagagtggcgaacgggtgagtaacacgtgggtaacctgcccatcagaaggggataacacttggaaacaggtgctaataccgtataacactattttccgcatggaagaaagttgaaaggcgcttttgcgtcactgatggatggacccgcggtgcattagctagttggtgaggtaacggctcaccaaggcaacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggatgagagtaaaatgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcgaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttaagttgggcac

SEQ ID NO: 201 - DNA, 1105 bp, Enterococcus sp.
  source 1..1105: mol_type = genomic DNA; note = 689B1_12EGalle
tcgaacgcttctttttccaccggagcttgctccaccggaraaagaggagtggcgaacgggtgagtaacacgtgggtaacctgcccatcagaaggggataacacttggaaacaggtgctaataccgtataacaatcraaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcact

SEQ ID NO: 202 - DNA, 1103 bp, Enterococcus sp.
  source 1..1103: mol_type = genomic DNA; note = 686B1_12ASB
acatgcaagtcgaacgctttttctttcaccggagcttgctccaccgaaagaaaaagagtggcgaacgggtgagtaacacgtgggtaacctgcccatcagaaggggataacacttggaaacaggtgctaataccgtataacactattttccgcatggaagaaagttgaaaggcgcttttgcgtcactgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggatgagagtagaacgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcgaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattta

SEQ ID NO: 203 - DNA, 1101 bp, Enterococcus sp.
  source 1..1101: mol_type = genomic DNA; note = 309B4_12EGalle
tgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattta

SEQ ID NO: 204 - DNA, 1100 bp, Enterococcus sp.
  source 1..1100: mol_type = genomic DNA; note = 630B2_12ASB
acatgcaagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatca

SEQ ID NO: 205 - DNA, 1087 bp, Enterococcus sp.
  source 1..1087: mol_type = genomic DNA; note = 631B2_12ASB
tgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgtta

SEQ ID NO: 206 - DNA, 1399 bp, unidentified
  source 1..1399: mol_type = genomic DNA; note = bacterium EM-2014-115
ggttacctcaccgacttcgggtgttacaaactctcgtggtgtgacgggcggtgtgtacaaggcccgggaacgtattcaccgcggcgtgctgatccgcgattactagcgattccggcttcatgcaggcgagttgcagcctgcaatccgaactgagagaagctttaagagattagcttagcctcgcgacttcgcaactcgttgtacttcccattgtagcacgtgtgtagcccaggtcataaggggcatgatgatttgacgtcatccccaccttcctccggtttgtcaccggcagtcttgctagagtgcccaactgaatgatggcaactaacaataagggttgcgctcgttgcgggacttaacccaacatctcacgacacgagctgacgacaaccatgcaccacctgtcactttgcccccgaaggggaagctctatctctagagtggtcaaaggatgtcaagacctggtaaggttcttcgcgttgcttcgaattaaaccacatgctccaccgcttgtgcgggcccccgtcaattcctttgagtttcaaccttgcggtcgtactccccaggcggagtgcttaatgcgttagctgcagcactgaagggcggaaaccctccaacacttagcactcatcgtttacggcgtggactaccagggtatctaatcctgtttgctccccacgctttcgagcctcagcgtcagttacagaccagagagccgccttcgccactggtgttcctccatatatctacgcatttcaccgctacacatggaattccactctcctcttctgcactcaagtctcccagtttccaatgaccctccccggttgagccgggggctttcacatcagacttaagaaaccgcctgcgctcgctttacgcccaataaatccggacaacgcttgccacctacgtattaccgcggctgctggcacgtagttagccgtggctttctggttagataccgtcaagggatgaacagttactctcatccttgttcttctctaacaacagagttttacgatccgaaaaccttcttcactcacgcggcgttgctcggtcagactttcgtccattgccgaagattccctactgctgcctcccgtaggagtttgggccgtgtctcagtcccaatgtggccgatcaccctctcaggtcggctatgcatcgtggccttggtgagccgttacctcaccaactagctaatgcaccgcgggtccatccatcagcgacacccgaaagcgcctttcaaatcaaaaccatgcggttttgattgttatacggtattagcacctgtttccaagtgttatccccttctgatgggcaggttacccacgtgttactcacccgttcgccactcctctttttccggtggagcaagctccggtggaaaaagaag

SEQ ID NO: 207 - DNA, 1321 bp, Enterococcus hirae
  source 1..1321: mol_type = genomic DNA; note = 8
tggcggcgtgcctaatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccgga

SEQ ID NO: 208 - DNA, 1138 bp, Enterococcus sp.
  source 1..1138: mol_type = genomic DNA; note = 930B6_12ESB
acatgcaagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaac

SEQ ID NO: 209 - DNA, 1487 bp, unidentified
  source 1..1487: mol_type = genomic DNA; note = Prior Sequence 28
agagtttgaatcatggctcaggacgaacgctggcggcgtgcctaatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgat

SEQ ID NO: 210 - DNA, 1350 bp, unidentified
  source 1..1350: mol_type = genomic DNA; note = Prior Sequence 29
ccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtgtatcaca

SEQ ID NO: 211 - DNA, 1436 bp, unidentified
  source 1..1436: mol_type = genomic DNA; note = Prior Sequence 30
catgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaagg

SEQ ID NO: 212 - DNA, 1475 bp, unidentified
  source 1..1475: mol_type = genomic DNA; note = Prior Sequence 31
acgacgctggcggcgtgcctatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtggatagagactggag

SEQ ID NO: 213 - DNA, 1467 bp, unidentified
  source 1..1467: mol_type = genomic DNA; note = Prior Sequence 32
ctggcggcgtgctatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtggatagagactttg

SEQ ID NO: 214 - DNA, 1478 bp, unidentified
  source 1..1478: mol_type = genomic DNA; note = Prior Sequence 33
ggagacgctggcggcgtgcctaatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtggatagagattggggg

SEQ ID NO: 215 - DNA, 1469 bp, unidentified
  source 1..1469: mol_type = genomic DNA; note = Prior Sequence 34
ctggcggcgtgcgtatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtggatagagactgggg

SEQ ID NO: 216 - DNA, 1468 bp, unidentified
  source 1..1468: mol_type = genomic DNA; note = Prior Sequence 35
ctggcggcgtgctaatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtggatagagactggt

SEQ ID NO: 217 - DNA, 1477 bp, unidentified
  source 1..1477: mol_type = genomic DNA; note = Prior Sequence 36
gggagacgctggcggcgtgcctatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtggatagagactgggg